(19) United States
(12) Patent Application Publication
(10) Pub. No.: US 2008/0119515 A1
(43) Pub. Date: May 22, 2008

(54) Heterocyclic Kinase Inhibitors: Methods of Use and Synthesis

(76) Inventors: M Arshad Siddiqui, Newton, MA (US); David B. Belanger, Cambridge, MA (US); Chaoyang Dai, Acton, MA (US); Lianyun Zhao, Dorchester, MA (US)

Correspondence Address: Palaiyur S Kalyanaraman;Schering-Plough Corporation, 2000 Galloping Hill Road, Kenilworth, NJ, 07033, US

(21) Appl. No.: 10/547,959

(22) PCT Filed: Mar. 10, 2004
(86) PCT No.: PCT/US04/07286

§ 371 Date: May 9, 2007

Related U.S. Application Data
(60) Provisional application No. 60/453,457, filed on Mar. 10, 2003.
(60) Provisional application No. 60/460,910, filed on Apr. 7, 2003.
(60) Provisional application No. 60/463,026, filed on Apr. 16, 2003.
(60) Provisional application No. 60/502,710, filed on Sep. 12, 2003.

(51) Int. Cl. A61K 31/47; A61P 19/02; C07D 215/00

(52) U.S. Cl. 514/312; 514/313; 546/152; 546/157; 546/159

(57) ABSTRACT

Inhibitors of kinases, compositions including the inhibitors, and methods of using the inhibitors and inhibitor compositions are described. The inhibitors and compositions including them are useful for treating disease or disease symptoms. The invention also provides for methods of making kinase inhibitor compounds, methods of inhibiting kinase activity, and methods for treating disease or disease symptom.

### CROSS-REFERENCE TO RELATED APPLICATIONS

This application claims priority to U.S. Provisional Application Ser. No. 60/453,457, filed on Mar. 10, 2003, U.S. Provisional Application Ser. No. 60/460,910, filed on Apr. 7, 2003, U.S. Provisional Application Ser. No. 60/463,025, filed on Apr. 15, 2003, and U.S. Provisional Application Ser. No. 60/502,710, filed Sep. 12, 2003, each of which is incorporated by reference herein in its entirety.

### BACKGROUND

Kinases are a class of enzymes that function in the catalysis of phosphoryl transfer. Protein kinases participate in the signaling events, which control the activation, growth and differentiation of cells in response to extracellular mediators and to changes in the environment. In general, kinases fall into several groups; those that preferentially phosphorylate serine and/or threonine residues are generally referred to as serine/threonine kinases and those that preferentially phosphorylate tyrosine residues are generally referred to as tyrosine kinases [S. K Hanks and T. Hunter, FASEB J., 1995, 9, 576-596]. The tyrosine kinases include membrane-spanning growth factor receptors such as EGFR (S. Iwashita and M. Kobayashi, Cellular Signaling, 1992, 4, 123-132), and cytosolic non-receptor kinases such as Lck, ZAP-70 and Syk kinases (C. Chan et. Al., Ann. Rev. Immun., 1994, 12, 555-592).

Inappropriately high protein kinase activity has been implicated in many diseases resulting from abnormal cellular function. This might arise either directly or indirectly, for example by failure of the proper control mechanisms for the kinase, related for example to mutation, over-expression or inappropriate activation of the enzyme; or by the over- or underproduction of cytokines or growth factors also participating in the transduction of signals upstream or downstream of the kinase. In all of these instances, selective inhibition of the kinase could have a beneficial effect.

T cells plays a key role in transplant rejection, autoimmune diseases and the initiation of inflammatory responses and are thus a primary target for pharmaceutical intervention in these indications. The activation of T cells is a complex process that results in cell growth and differentiation. The engagement of the T cell receptor on mature peripheral T cells initiates multiple intracellular signals that lead to cellular proliferation and the acquisition of complex functions. The biochemical mechanisms that couple receptor binding to these intracellular events have been investigated (J. E. M., Van Leeuwen, and L. E. Samelson, Current Opin. Immun. 1999, 11, 242-248). The Syk family of tyrosine kinases, comprising Syk and ZAP-70, play a role in the initiation and the amplification of receptor signal transduction (D. H. Chu et. al., Immunol. Rev. 1998, 165, 167-180). ZAP-70 is expressed solely in T cells and NK cells. Syk is found in B cells, mast cells, neutrophils, macrophages, and platelets and is involved in B cell receptor and Fc receptor signal transduction. Thus, kinase inhibitors of ZAP-70 and Syk have potential therapeutic benefits for treating diseases resulting from activation and differentiation of T cells, NK cells, B cells, mast cells, neutrophils, macrophages, and platelets.

### SUMMARY

The invention relates to novel compounds and compositions including those compounds, as well as methods of using and making the compounds. The compounds are heterocyclic compounds that are useful in therapeutic applications, including modulation of disease or disease symptoms in a subject (e.g., mammal, human, dog, cat, horse). The compounds (including stereoisomers thereof) are created either singly or in a combinatorial fashion to give structurally, and stereochemically diverse libraries of compounds. The compounds are useful as ZAP-70 and Syk inhibitors through their binding to these receptors.

In one aspect, the invention features a compound of the formula (I)

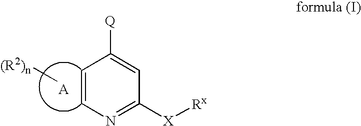

wherein;

A forms a benzene, pyridine, pyrimidine, thiophene, pyrrole, imidazole, pyrrazole, thiazole, or oxazole ring;

X is O, S, NR³, N(R³)N(R³), C(O), N(R⁵)C(O), C(O)NR⁵, or alkyl;

R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;

each R² is independently H, NR⁵₂, alkyl, cycloalkyl, alkenyl, alkynyl, heteroaryl, heterocyclyl, aryl halo, acetyl, arylalkyl, heteroarylalkyl, arylalkenyl heteroarylalkenyl, arylalkynyl, heteroarylalkynyl, hydroxy, alkoxy, aryloxy, or heteroaryloxy, each of which is optionally substituted with 1-4 R⁷; wherein R² is not Me when X—R^{X} is Me and Q is NHaryl substituted with heterocycyl; and wherein R² is not acetyl when X—R^{X} is arylalkenyl;

n is 0-3;

each R³ is independently H, alkyl, R⁵O-alkyl or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;

each R⁹ is independently aryl, heterocyclyl, heteroaryl, arylalkyl, or heteroarylalkyl, each of which is optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and

each R¹¹ is independently arylalkyl, heteroarylalkyl, cycloalkyl, or heterocyclyl.

In some instances, A forms a benzene ring.

In another aspect, the invention features a compound of formula (II)

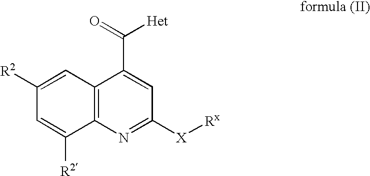

wherein;

X is O, S, NR³, N(R³)N(R³), C(O), N(R⁵)C(O), C(O)NR⁵, or alkyl;

R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;

each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷;

each R^{2′} is independently H, halo, NH₂, alkyl, OH, C(O)Me, aryl, heteroaryl;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Het is heterocyclyl optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R⁵R¹¹-alkyl, R⁵O-alkyl, R¹¹, heteroaryl, heterocyclyl, or heterocyclylalkyl, each of which is optionally substituted with alkyl or OR⁵; and

each R¹¹ is independently arylalkyl, heteroarylalkyl, cycloalkyl, or heterocyclyl.

In some instances, Het is attached through a ring-nitrogen atom.

In some instances, R^{2′} is H.

In some instances, Het is attached through a ring-nitrogen atom; X is NR³, or alkyl; and R^{X} is cycloalkyl, aryl, heteroaryl, arylalkyl, heteroarylalkyl, or heterocyclyl wherein R^{X} is optionally substituted with 1-4 R⁴; and R^{2′} is H.

In some instances, Het is

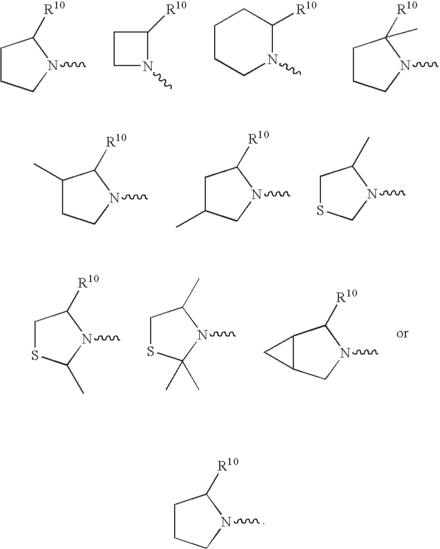

In other instances, Het is

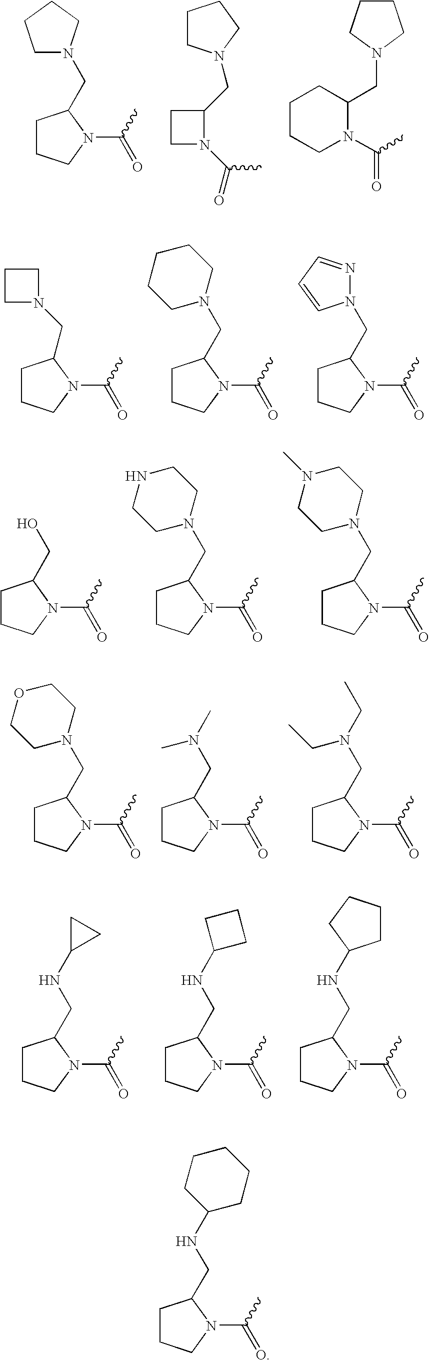

In another aspect, the invention features a compound of formula (III),

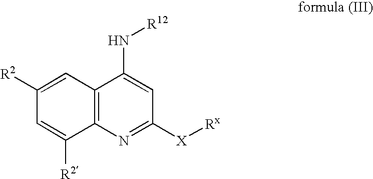

wherein,

X is O, S, NR³, N(R³)N(R³), C(O), N(R)C(O), C(O)NR⁵, or alkyl;

R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;

each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when X—R^{X} is Me and R¹² is aryl substituted with heterocyclyl;

each R^{2′} is independently H, halo, NH₂, alkyl, OH, C(O)Me, aryl, heteroaryl;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

each R¹² is independently aryl or heteroaryl, optionally substituted with 1-4 R¹³;

each R¹³ is independently heterocyclyl optionally substituted with alkyl or OR⁵.

In some instances R^{2′} is H.

In some instances R¹² is

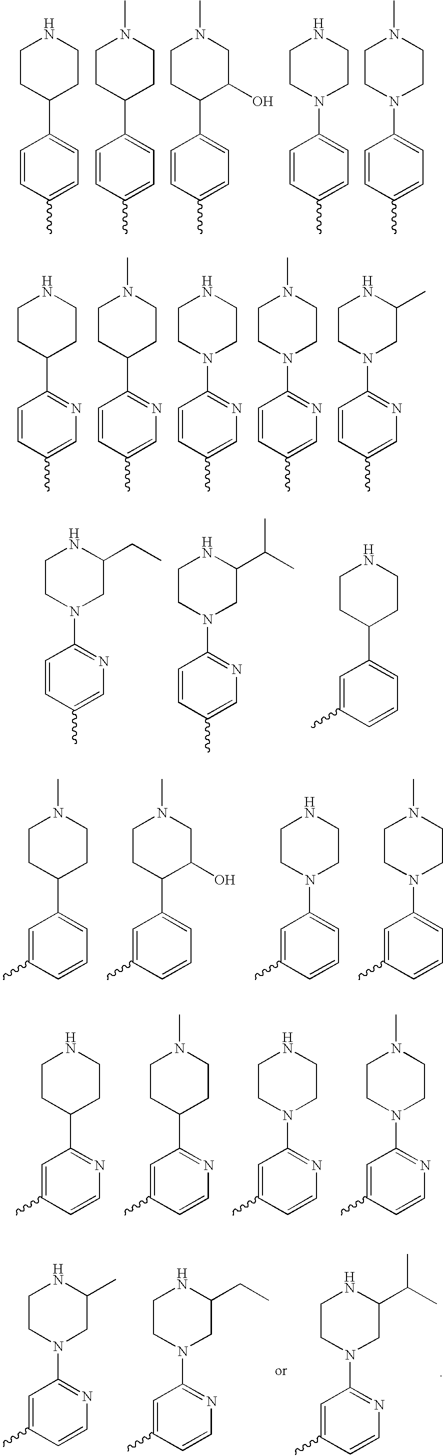

In another aspect, the invention features a compound of formula (IV)

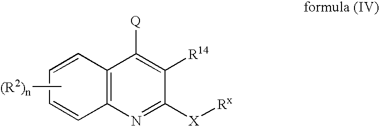

wherein,

X is O, S, NR³, N(R³)N(R³), C(O), N(R⁵)C(O), C(O)NR⁵, or alkyl;

R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;

each R² is independently H, NR⁵₂, alkyl, cycloalkyl, alkenyl, alkynyl heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when X—R^{X} is Me and Q is NHAryl substituted with heterocycyl; and wherein R² is not acetyl when X—R^{X} is arylalkenyl;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR², NR⁵R⁹, R⁹S-alkyl, alkyl, or heterocyclyl, wherein each alkyl or heterocyclyl is optionally substituted with 1-4 R¹⁰;

each R⁹ is independently aryl, heterocyclyl, heteroaryl, arylalkyl, or heteroarylalkyl, each of which is optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵;

each R¹¹ is independently arylalkyl, heteroarylalkyl, cycloalkyl, or heterocyclyl; and

R¹⁴ is halo, NH₂, alkyl, OH, C(O)Me, aryl, heteroaryl, or C(O)NHR⁵.

In yet another aspect, the invention features a compound of formula (V)

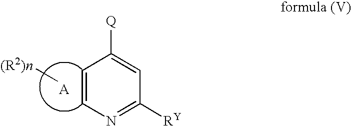

wherein;

A forms a benzene, pyridine, pyrimidine, thiophene, pyrrole, imidazole, pyrrazole, thiazole, or oxazole ring;

R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;

each R² is independently H, NR², alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when R¹ is halo, indole substituted with halo, phenyl, or phenyl substituted with halo; wherein R² is not isopropyl when R^{Y} is phenyl substituted with halo; wherein R² is not alkynyl substituted with heterocyclyl when R¹ is indole substituted with halo; wherein R² is not phenyl when R^{Y} is H, wherein R² is not halo when R^{Y} is indole substituted with halo or phenyl substituted with halo; and wherein R² is not acetyl when R^{Y} is phenyl or substituted phenyl;

n is 0-3;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;

each R⁹ is independently aryl, heterocyclyl, heteroaryl, arylalkyl, or heteroarylalkyl, each of which is optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and

each R¹¹ is independently arylalkyl, heteroarylalkyl, cycloalkyl, or heterocyclyl.

In some instances A forms a benzene ring.

In one aspect, the invention features a compound of formula (VI), wherein

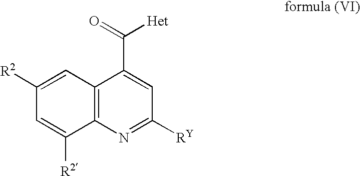

wherein,

R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;

each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when R^{Y} is halo, indole substituted with halo, phenyl, or phenyl substituted with halo; wherein R² is not isopropyl when R^{Y} is phenyl substituted with halo;

each R^{2′} is independently H, halo, NH₂, alkyl, OH, C(O)Me, aryl, heteroaryl;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl each of which is optionally substituted with 1-4 R⁸;

each R⁸ is independently OR⁵ or alkyl;

Het is heterocyclyl optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and

each R¹¹ is independently arylalkyl, heteroarylalkyl, cycloalkyl, or heterocyclyl.

In some instances, Het is attached through a ring-nitrogen atom; R² is not H; and R^{2′} is H.

In some instances, Het is

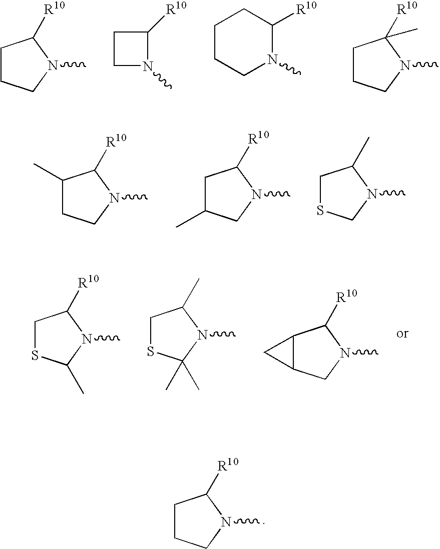

In some instances Het is

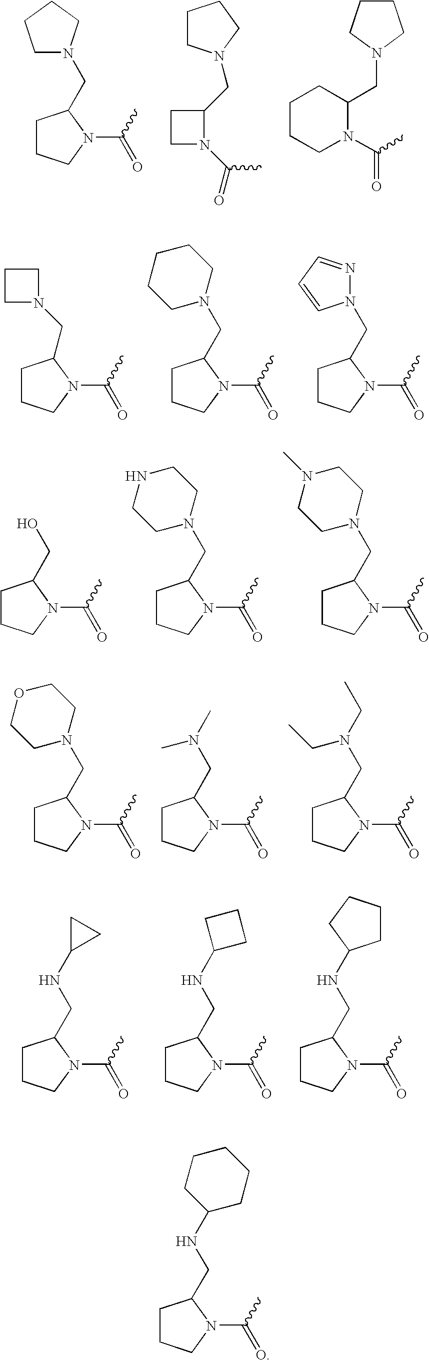

In another aspect, the invention features a compound of formula (VII)

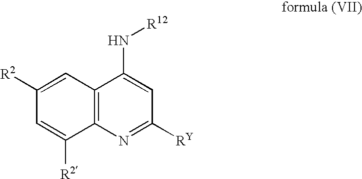

wherein;

R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;

each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not phenyl when R^{Y} is H;

each R^{2′} is independently H, halo, NH₂, alkyl, OH, C(O)Me, aryl, heteroaryl;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

each R¹² is independently aryl or heteroaryl, optionally substituted with 1-4 R¹³;

each R¹³ is independently heterocyclyl optionally substituted with alkyl or OR⁵.

In some instances R² is not H; and R^{2′} is H.

In some instances R¹² is

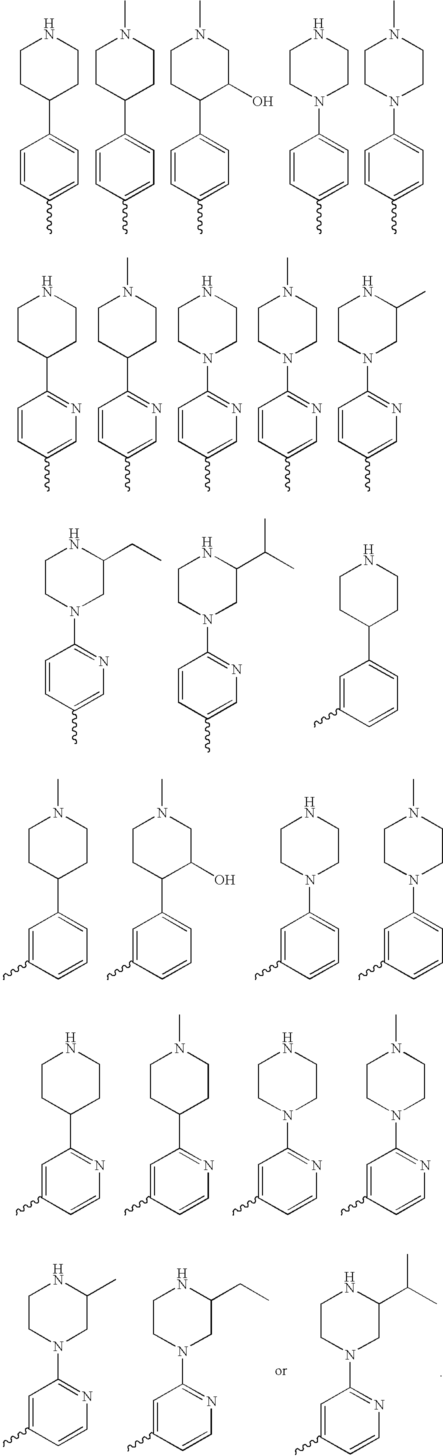

In one aspect, the invention features a compound of formula (VIII),

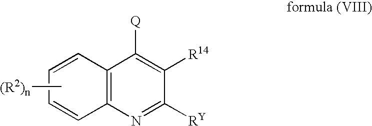

wherein,

R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;

each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when R^{Y} is halo, indole substituted with halo, or phenyl optionally substituted with halo; wherein R² is not isopropyl when R^{Y} is phenyl substituted with halo; wherein R² is not alkynyl substituted with heterocyclyl when R^{Y} is indole substituted with halo; wherein R² is not phenyl when R^{Y} is H; and wherein R² is not acetyl when R^{Y} is phenyl or substituted phenyl;

each n is 0-3;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR¹², SO₂NR⁵R⁹, NR⁵₂, NR⁵R⁹, R⁹S-alkyl, alkyl, or heterocyclyl wherein each alkyl or heterocyclyl is optionally substituted with 1-4 R¹⁰;

each R⁹ is independently aryl, heterocyclyl, heteroaryl, arylalkyl, or heteroarylalkyl, each of which is optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R¹¹-alkyl R⁵O-alkyl, R¹¹, heteroaryl, heterocyclyl, or heterocyclylalkyl, each of which is optionally substituted with alkyl or OR⁵;

each R¹¹ is independently arylalkyl, heteroarylalkyl, cycloalkyl, or heterocyclyl; and

R¹⁴ is halo, NH₂, alkyl, OH, C(O)Me, aryl, heteroaryl, or C(O)NHR⁵.

In some instances, n is 1 and R² is not H.

In another aspect, the invention features the compound of formula (XIV),

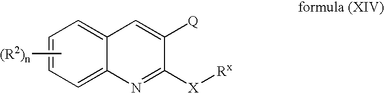

wherein,

X is O, S, NR³, N(R³)N(R³), C(O), N(R⁵)C(O), C(O)N(R⁵, or alkyl;

R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵-alkyl, (R⁵)₃Si, acyl, wherein R^{X} is optionally substituted with 1-4 R⁴;

each R² is independently H, NR⁵₂, alkyl, cycloalkyl, alkenyl, alkyl, heteroaryl, heterocyclyl, aryl, halo, arylalkyl, heteroarylalkyl, arylalkenyl, heteroarylalkenyl, arylalkynyl, heteroarylalkynyl, aryloxy, or heteroaryloxy, each of which is optionally substituted with 1-4 R⁷;

n is 0-3;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R⁵)C(O)R⁵, C(O)R⁵, OCF₃, SCF₃, NR¹², C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;

each R⁹ is independently aryl, heterocyclyl, heteroaryl, arylalkyl, or heteroarylalkyl, each of which is optionally substituted with 1-4 R¹⁰;

each R¹⁰ is independently alkyl, CF₃, C(NH)R⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and

each R¹¹ is independently arylalkyl, heteroarylalkyl, cycloalkyl, or heterocyclyl.

In yet another aspect, the invention features a method of making a compound of formula (II)

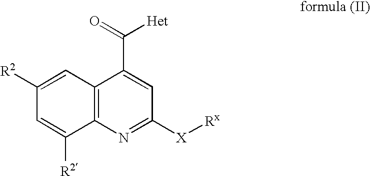

wherein X, R^{X}, R², R^{2′}, and Het are as defined below. The method includes;

treating the compound of formula (IX) with malonic acid, to provide a ring expansion compound of formula (X);

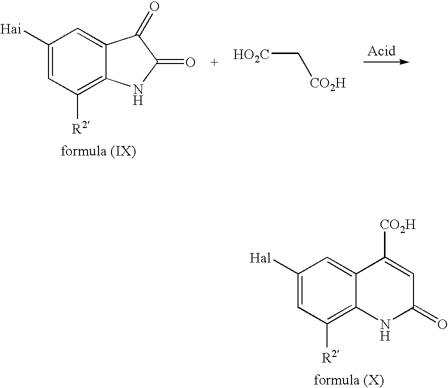

coupling the compound of formula (X) with a Pd catalyst and a compound of formula (XI) to provide a compound of formula (XII);

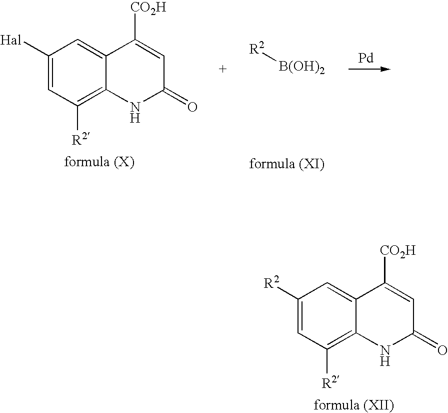

treating the compound of formula (XII) with POCl₃ to provide the chloride of formula (XIII), and coupling the carboxylic acid of formula (XII) with an amine of formula Het-H to provide the compound of formula (XIII); and

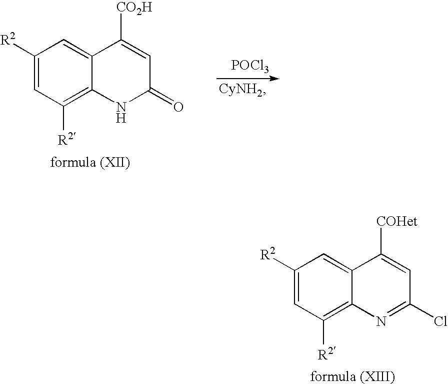

coupling the compound of formula (XII) with one or more coupling agents

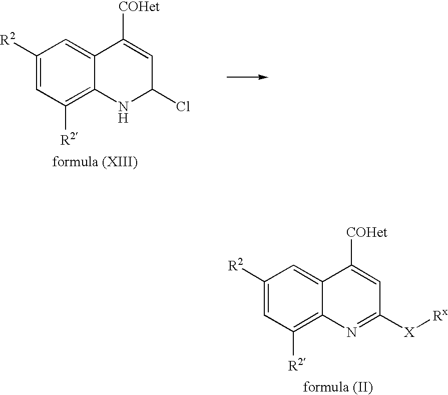

to provide a compound of formula (I), wherein for formulae II and IX to XIII,

X is O, S, NR³, N(R³)N(R³), C(O), N(R⁵)C(O)R⁵, C(O)NR⁵, or alkyl;

R^{X} is H, cycloallyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;

each R² is independently NR⁵₂, alkyl, cycloalkyl, alkenyl, alkynyl, heteroaryl, heterocyclyl, aryl, halo, arylalkyl, heteroarylalkyl, arylalkenyl, heteroarylalkenyl, arylalkynyl, heteroarylalkynyl, aryloxy, or heteroaryloxy, each of which is optionally substituted with 1-4 R⁷;

R^{2′} is H, halo, NH₂, alkyl, OH, C(O)Me, aryl, heteroaryl;

each R³ is independently H, alkyl, R⁵O-alkyl, or arylalkyl;

each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;

each R⁵ is independently H, or alkyl;

each R⁶ is independently halo, OH, CF₃, alkyl, alkyloxy, N(R⁵)-alkyl, heteroaryl, heteroarylalkyl, or heterocyclyl;

each R⁷ is independently halo, CN, OR⁵, CF₃, N(R)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;

each R⁸ is independently OR⁵ or alkyl;

Het is heterocyclyl optionally substituted with 1-4 R¹⁰;

each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and

each R¹¹ is independently arylalkyl, heteroarylalkyl, cycloalkyl, or heterocyclyl.

In some instances, the H of Het-His attached to a nitrogen.

In some instances, the coupling agent is H₂NR^{X}.

In some instances, the coupling agents are MgCl—R^{X}, and Pd.

In some instances the coupling agent is HSR^{X}.

In another aspect, the invention features a method of treating an autoimmune disorder in a subject including administering to the subject any of the compounds or compositions described herein.

In some instances, the method includes administering an additional therapeutic agent.

In some instances the autoimmune disorder is lupus.

In another aspect, the invention features a method of treating organ transplant rejection in a subject comprising administering to the subject any of the any of the compounds or compositions described herein.

In another aspect, the invention features a method of treating an inflammatory disorder in a subject comprising administering to the subject any of the compounds or compositions described herein.

In some instances the method includes administering an additional therapeutic agent. The additional therapeutic agent can be an analgesic, or a steroid.

In some instances, the inflammatory disorder is arthritis. The arthritis can be, for example, rheumatoid arthritis, rheumatoid spondylitis, gouty arthritis, traumatic arthritis, rubella arthritis, psoriatic arthritis, or osteoarthritis.

In some instances, the inflammatory disorder is inflammatory bowel disease or Crohn's disease.

In another aspect, the invention features a composition including any of the compounds described herein.

In some instances the composition can include a pharmaceutically acceptable carrier.

In some instance, the composition can include an additional therapeutic agent.

In another aspect, the invention features a library of the compounds of any of formulae (I)-(VIII).

In yet another aspect, the invention features a method of inhibiting IL-2 production in a subject including administering to the subject any of the compounds or compositions described herein.

In still another aspect, the invention features a method of modulating ZAP-70 or Syk in a subject including administering to the subject any of the compounds or compositions described herein.

In other aspects, the compounds, compositions, and methods delineated herein are those of any of the compounds of Table 1 herein.

The details of one or more embodiments of the invention are set forth in the accompanying drawings and the description below. Other features, objects, and advantages of the invention will be apparent from the description and drawings, and from the claims.

### DETAILED DESCRIPTION

The term “halo” refers to any radical of fluorine, chlorine, bromine or iodine. The term “alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. The term “lower alkyl” refers to a C₁-C₈ alkyl chain. In the absence of any numerical designation, “alkyl” is a chain (straight or branched) having 1 to 10 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl substituted with an amino. The term “mercapto” refers to an —SH radical. The term “thioalkoxy” refers to an —S-alkyl radical.

The term “alkenyl” refers to a hydrocarbon chain that may be a straight chain or branched chain having one or more carbon-carbon double bonds. The alkenyl moiety contains the indicated number of carbon atoms. For example, C₂-C₁₀ indicates that the group may have from 2 to 10 (inclusive) carbon atoms in it. The term “lower alkenyl” refers to a C₂-C₈ alkenyl chain. In the absence of any numerical designation, “alkenyl” is a chain (straight or branched) having 2 to 10 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂-C₁₀ indicates that the group may have from 2 to 10 (inclusive) carbon atoms in it. The term “lower alkynyl” refers to a C₂-C₈ alkynyl chain. In the absence of any numerical designation, “alkynyl” is a chain (straight or branched) having 2 to 10 (inclusive) carbon atoms in it.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkenyl” refers to an alkenyl substituted with an aryl. The term “arylalkynyl” refers to an alkynyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkenyl” refers to an alkenyl substituted with a heteroaryl. The term “heteroarylalkynyl” refers to an alkynyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. The term “heterocyclylalkyl” refers to an alkyl substituted with a heterocyclyl.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “sulfonyl” refers to a sulfur attached to two oxygen atoms through double bonds. An “alkylsulfonyl” refers to an alkyl substituted with a sulfonyl.

The term “substituents” refers to a group “substituted” on an alkyl, cycloalkyl, aryl, heterocyclyl, or heteroaryl group at any atom of that group. Suitable substituents include, without limitation, halo, hydroxy, mercapto, oxo, nitro, haloalkyl, alkyl, aryl, aralkyl, alkoxy, thioalkoxy, aryloxy, amino, alkoxycarbonyl, amido, carboxy, alkanesulfonyl, alkylcarbonyl, and cyano groups.

The term “Me” means methyl.

The term “Ac” means acetyl.

The term “dppf” means 1,1 bis(diphenylphosphino) ferrocene.

The term “Mops” means 3-(N-Morpholino) propanesulfonic acid.

The term “BSA” means bovine serum albumin.

The term “LAT” means a palmitoylated p36/38 kDa plasma membrane-associated protein expressed in all T lymphocytes and it is the physiologic substrate of activated ZAP-70.

The term “TMB” means 3,3′,5,5′-Tetramethylbenzidine.

The term “kinase mediated disorder” means a disorder wherein a protein kinase is involved in signaling, mediation, modulation, or regulation of the disease process or symptoms. Kinase mediated disorders are exemplified by the following classes of diseases and disorders: cancer, autoimmunological, metabolic, inflammatory, infection (bacterial, viral, yeast, fungal, etc.), diseases of the central nervous system, degenerative neural disease, allergy/asthma, dermatology, angiogenesis, neovascularization, vasculogenesis, cardiovascular, and the like.

TABLE 1
Representative compounds of the invention**

| Number | MOLSTRUCTURE |
|---|---|
| 1 | 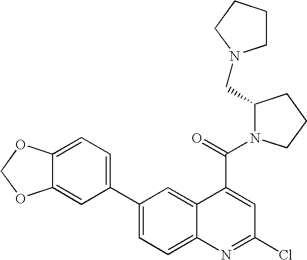 |
| 2 | 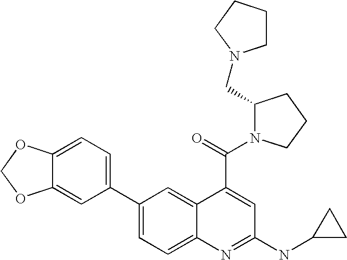 |
| 3 | 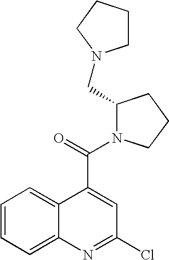 |
| 4 | 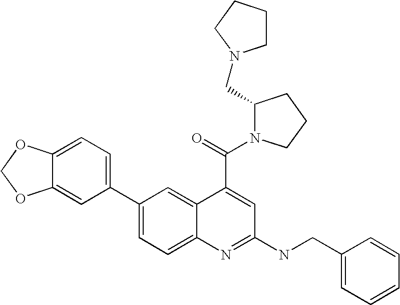 |
| 5 | 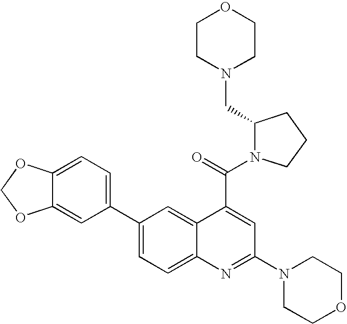 |
| 6 | 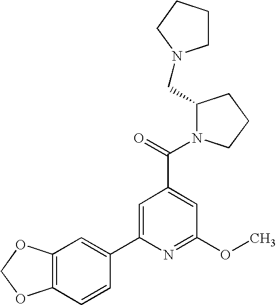 |
| 7 | 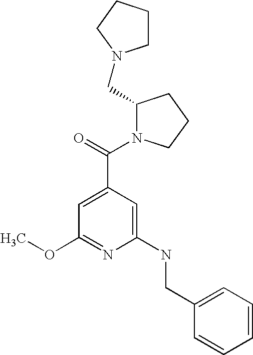 |
| 8 | 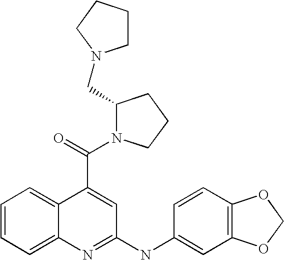 |
| 9 | 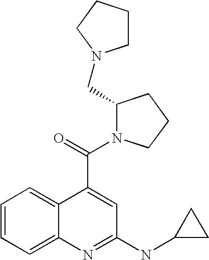 |
| 10 | 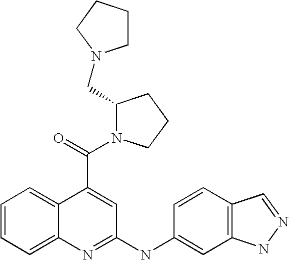 |
| 11 | 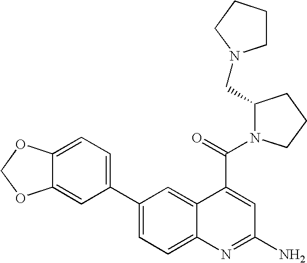 |
| 12 | 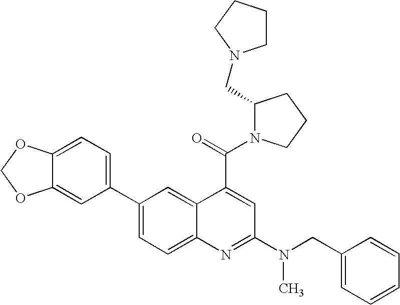 |
| 13 | 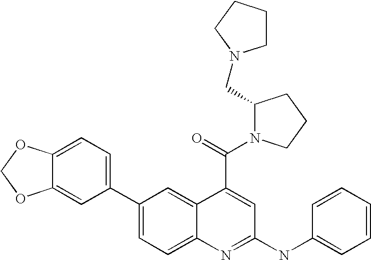 |
| 14 | 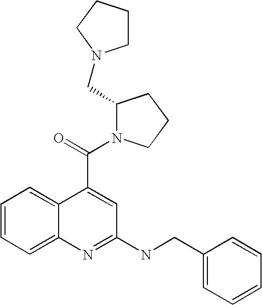 |
| 15 | 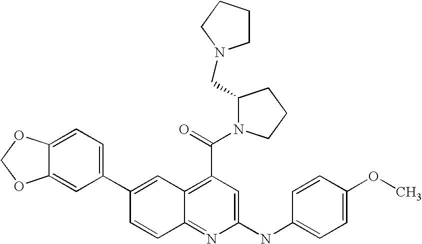 |
| 16 | 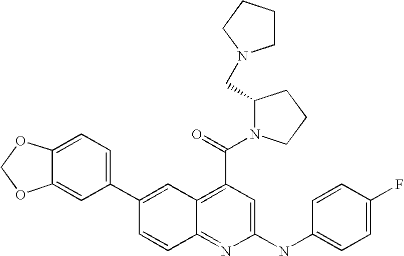 |
| 17 | 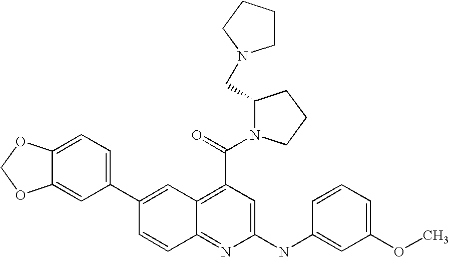 |
| 18 | 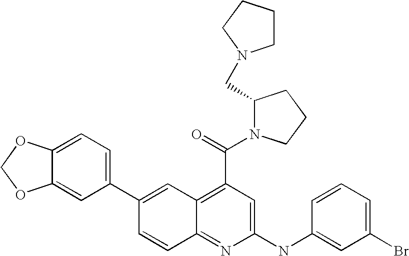 |
| 19 | 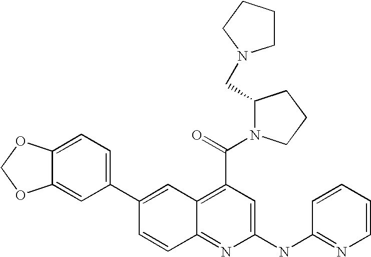 |
| 20 | 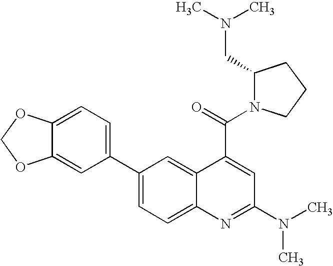 |
| 21 | 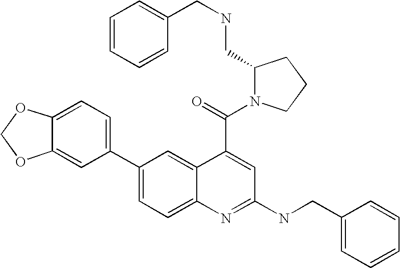 |
| 22 | 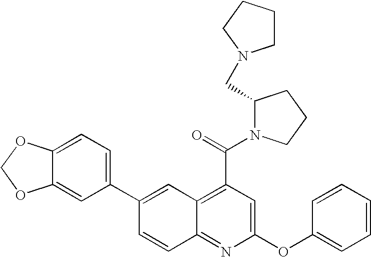 |
| 23 | 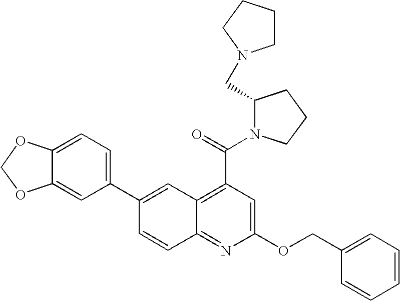 |
| 24 | 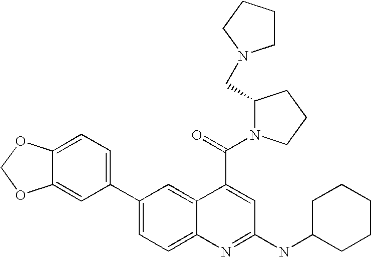 |
| 25 | 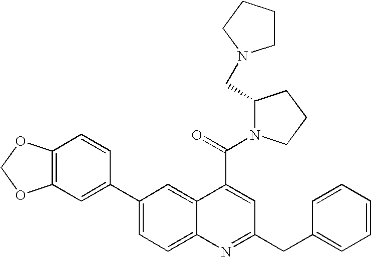 |
| 26 | 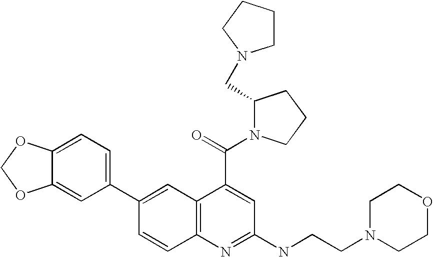 |
| 27 | 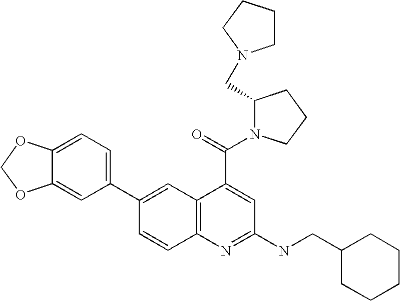 |
| 28 | 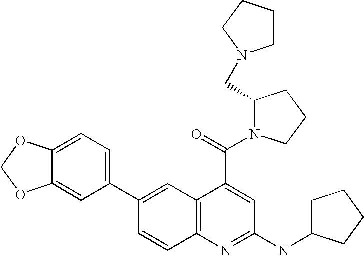 |
| 29 | 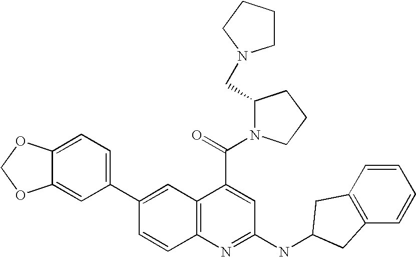 |
| 30 | 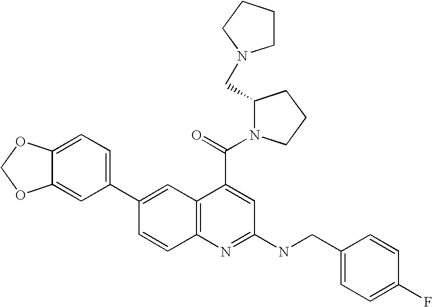 |
| 31 | 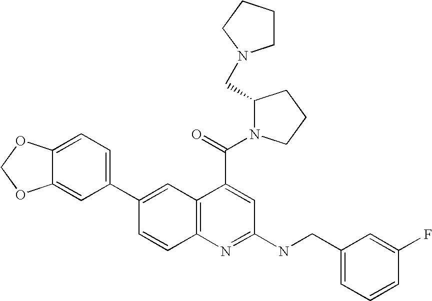 |
| 32 | 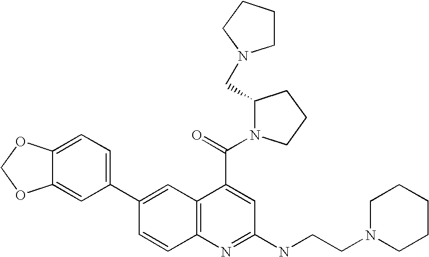 |
| 33 | 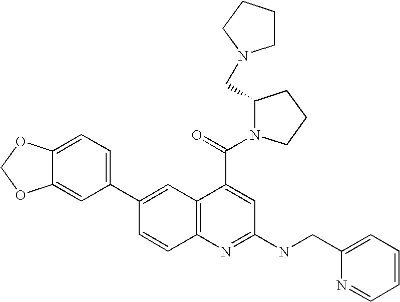 |
| 34 | 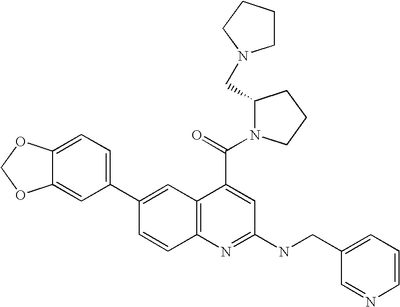 |
| 35 | 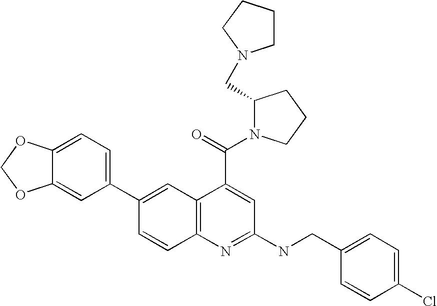 |
| 36 | 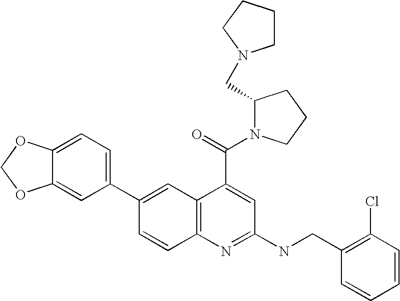 |
| 37 | 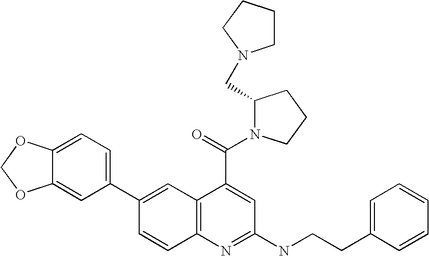 |
| 38 | 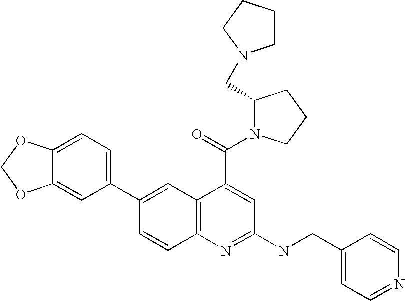 |
| 39 | 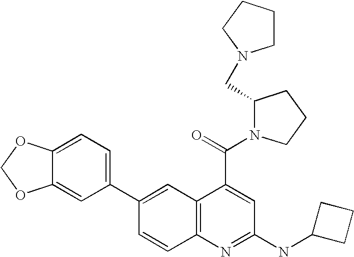 |
| 40 | 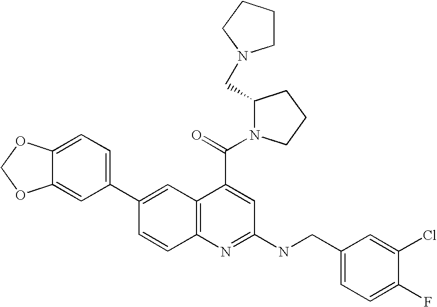 |
| 41 | 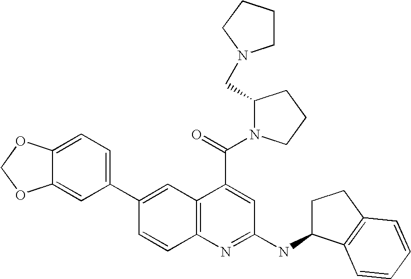 |
| 42 | 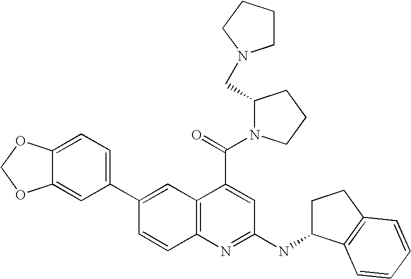 |
| 43 | 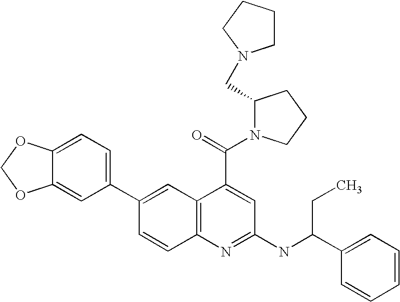 |
| 44 | 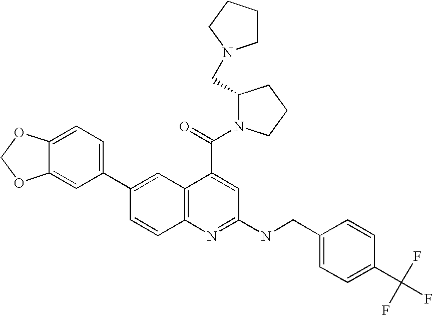 |
| 45 | 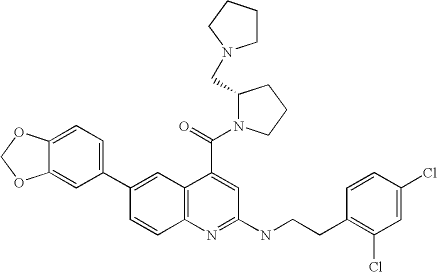 |
| 46 | 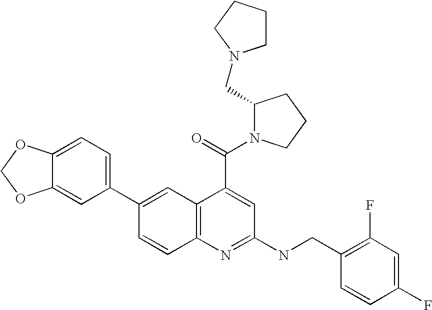 |
| 47 | 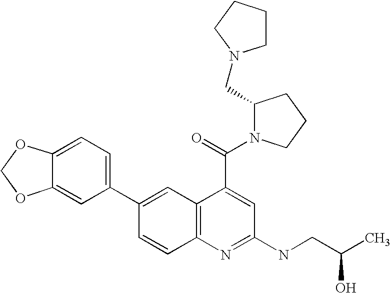 |
| 48 | 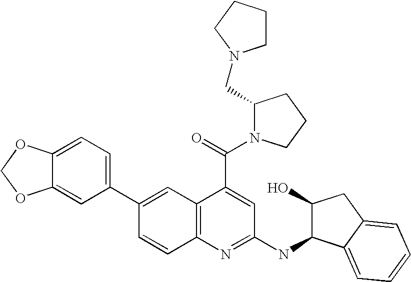 |
| 49 | 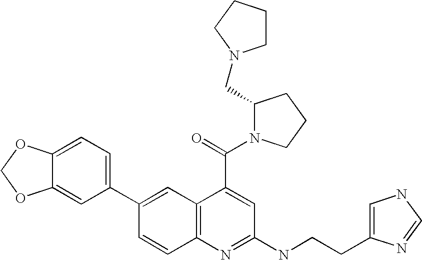 |
| 50 | 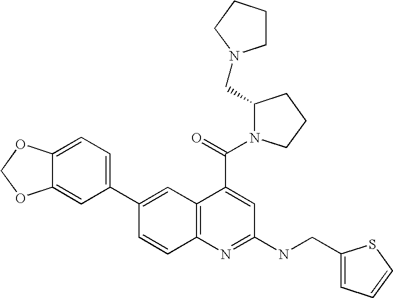 |
| 51 | 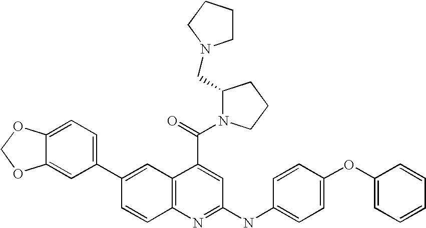 |
| 52 | 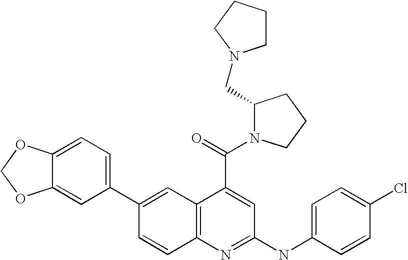 |
| 53 | 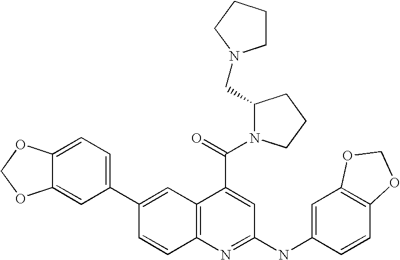 |
| 54 | 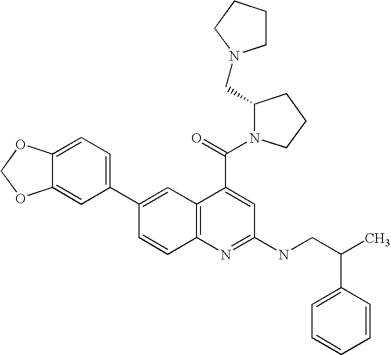 |
| 55 | 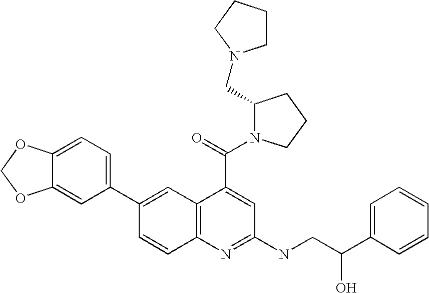 |
| 56 | 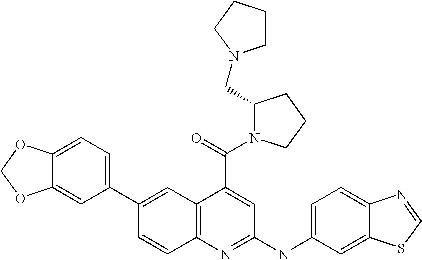 |
| 57 | 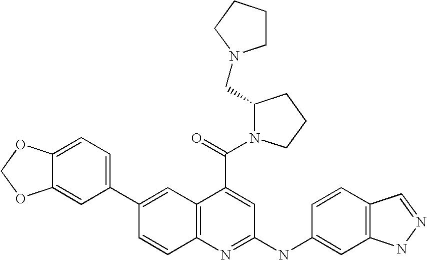 |
| 58 | 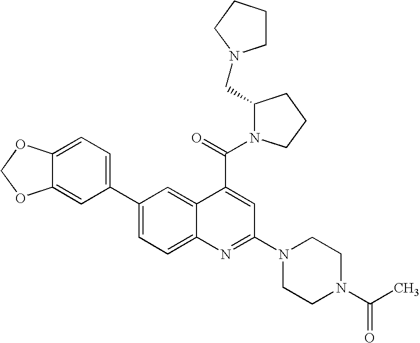 |
| 59 | 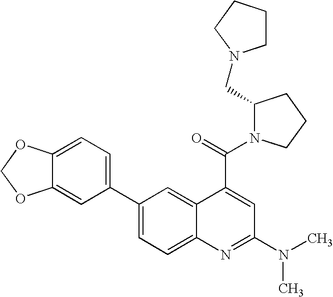 |
| 60 | 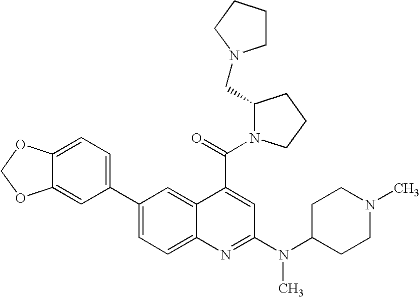 |
| 61 | 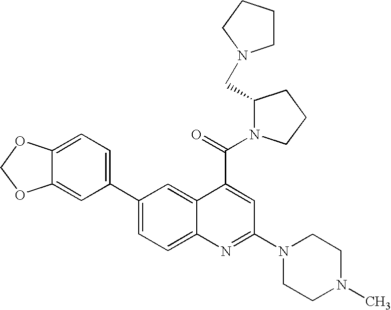 |
| 62 | 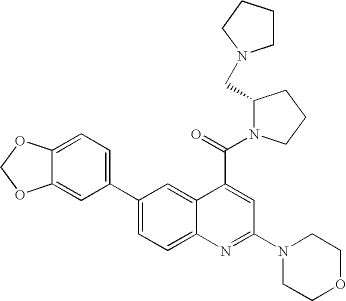 |
| 63 | 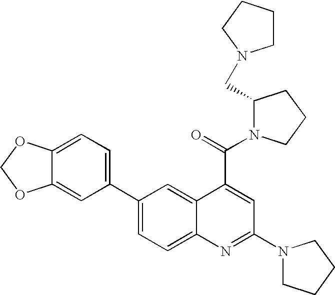 |
| 64 | 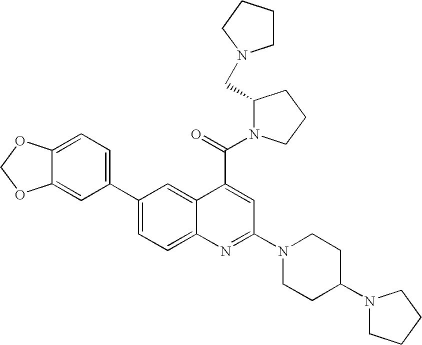 |
| 65 | 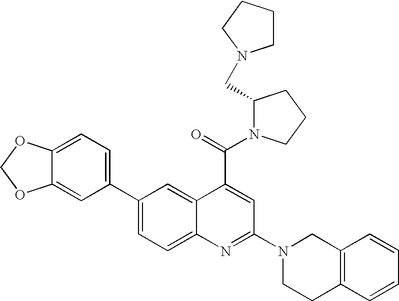 |
| 66 | 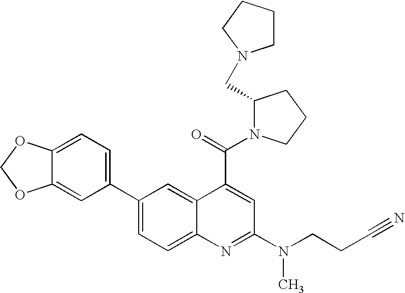 |
| 67 | 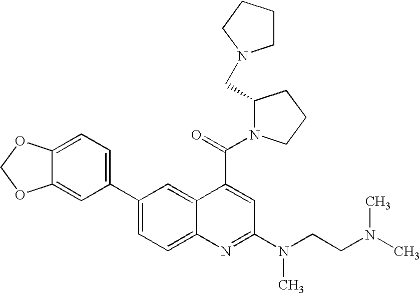 |
| 68 | 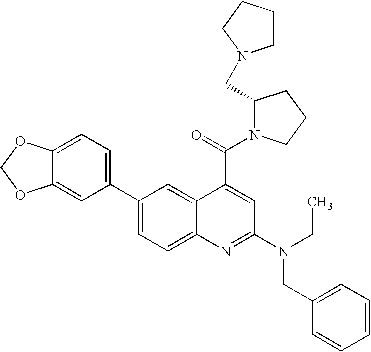 |
| 69 | 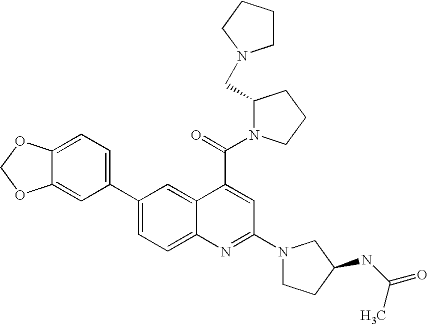 |
| 70 | 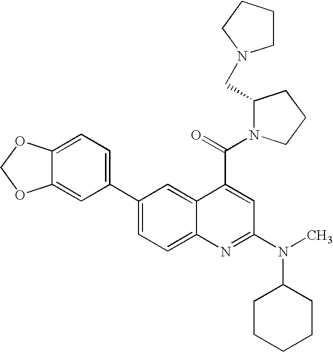 |
| 71 | 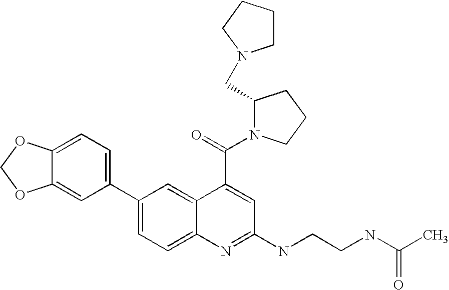 |
| 72 | 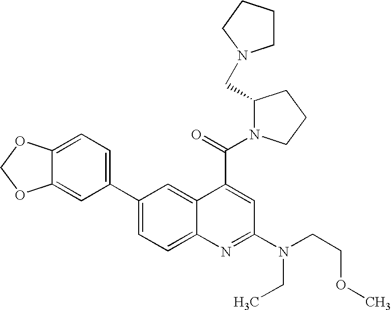 |
| 73 | 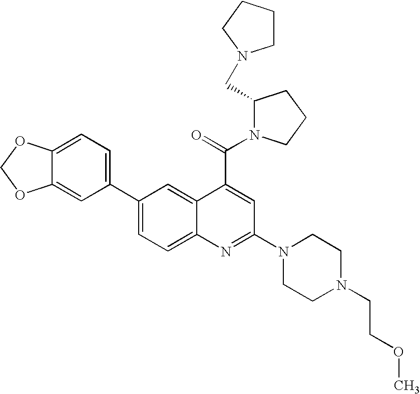 |
| 74 | 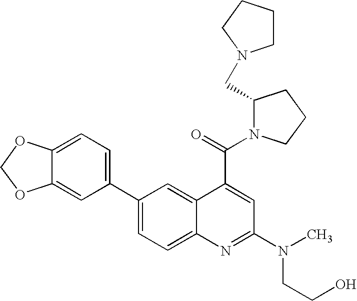 |
| 75 | 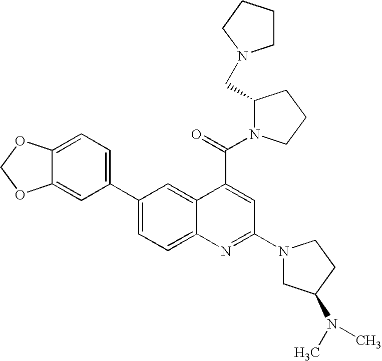 |
| 76 | 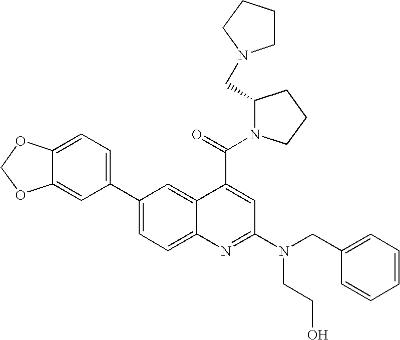 |
| 77 | 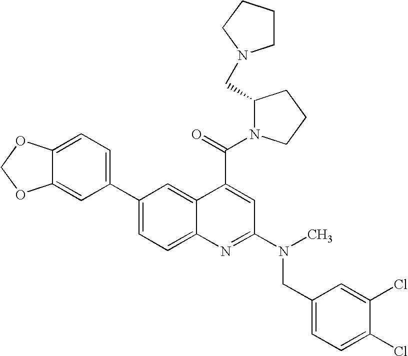 |
| 78 | 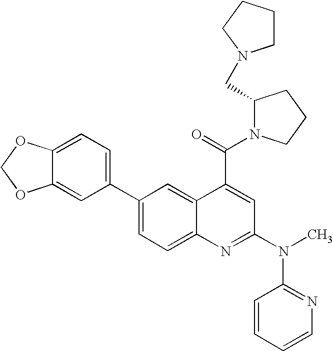 |
| 79 | 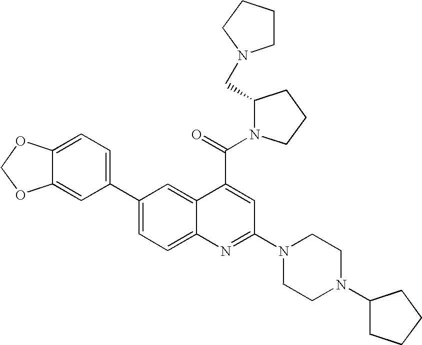 |
| 80 | 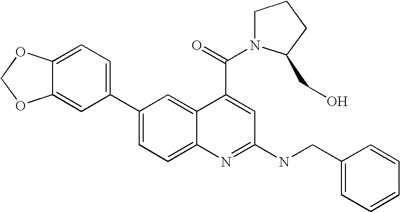 |
| 81 | 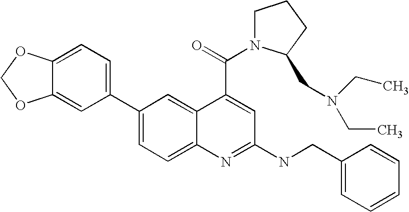 |
| 82 | 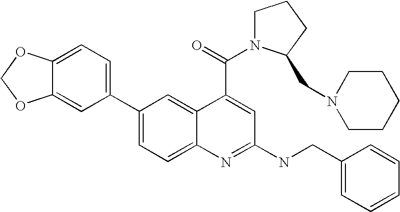 |
| 83 | 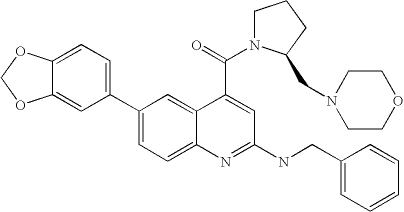 |
| 84 | 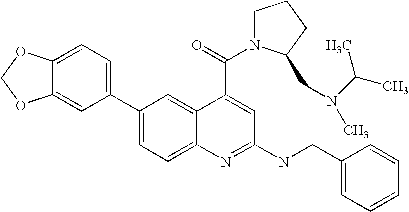 |
| 85 | 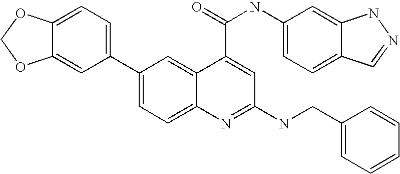 |
| 86 | 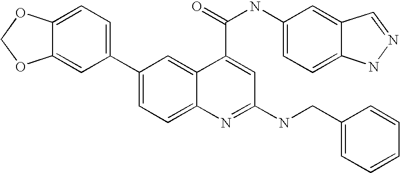 |
| 87 | 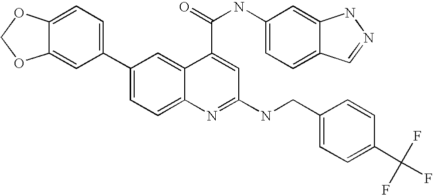 |
| 88 | 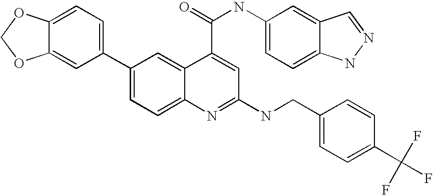 |
| 89 | 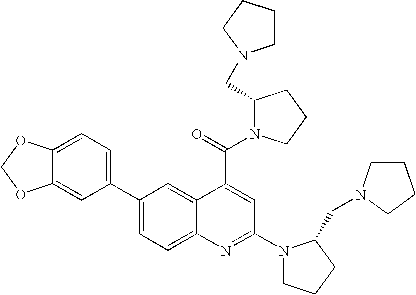 |
| 90 | 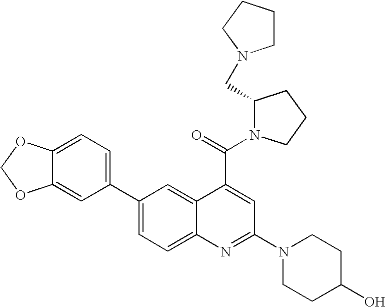 |
| 91 | 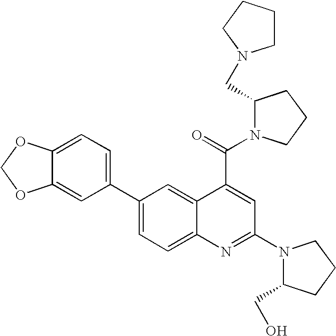 |
| 92 | 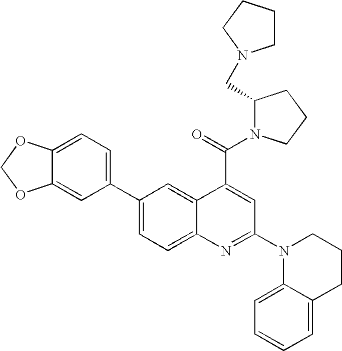 |
| 93 | 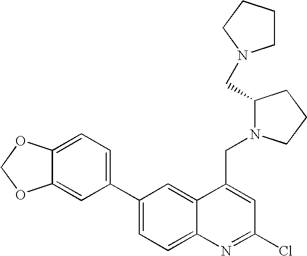 |
| 94 | 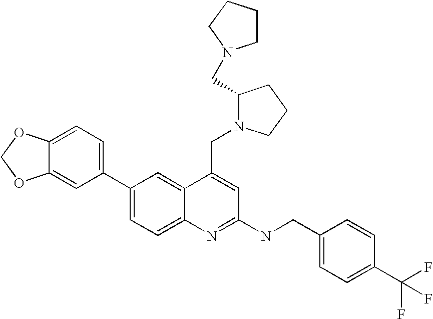 |
| 95 | 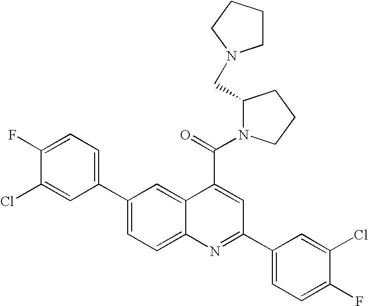 |
| 96 | 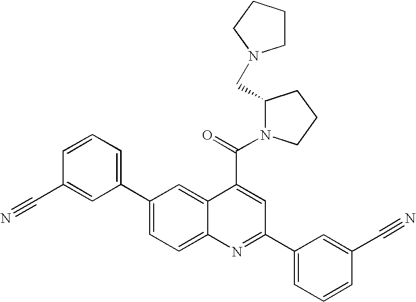 |
| 97 | 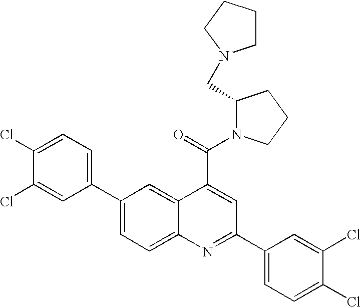 |
| 98 | 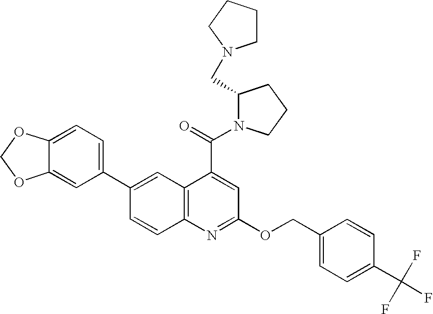 |
| 99 | 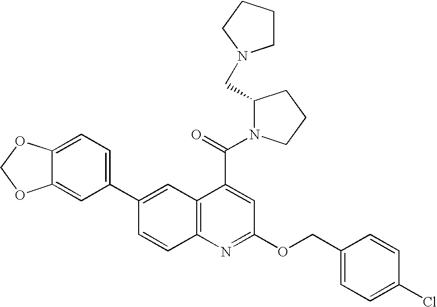 |
| 100 | 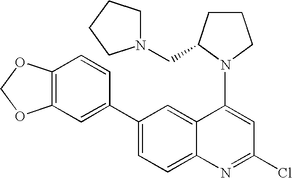 |
| 101 | 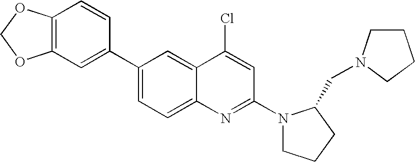 |
| 102 | 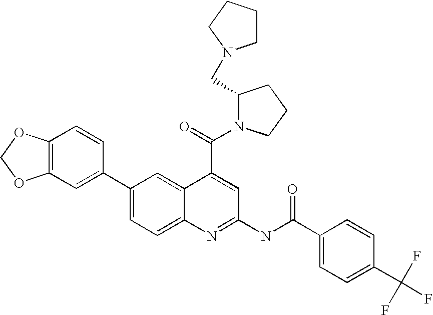 |
| 103 | 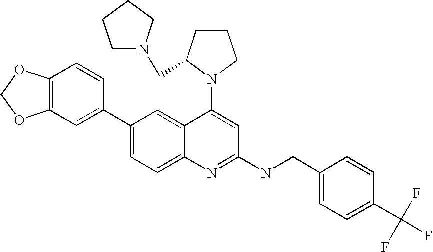 |
| 104 | 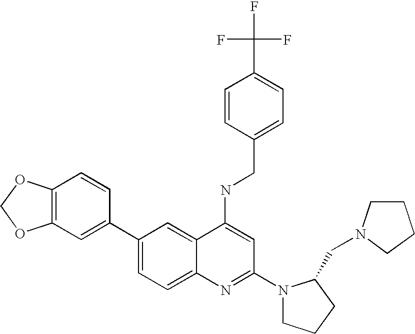 |
| 105 | 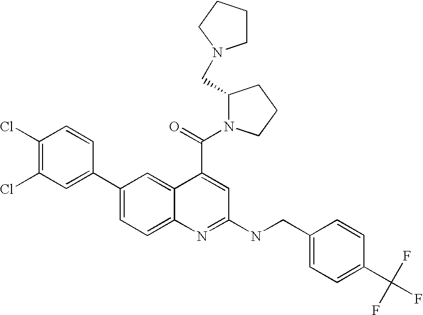 |
| 106 | 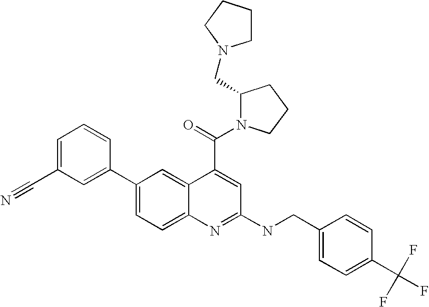 |
| 107 | 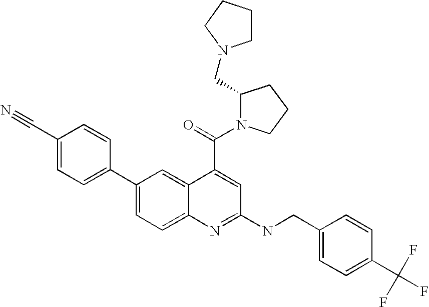 |
| 108 | 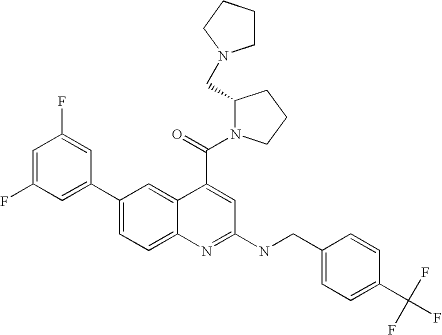 |
| 109 | 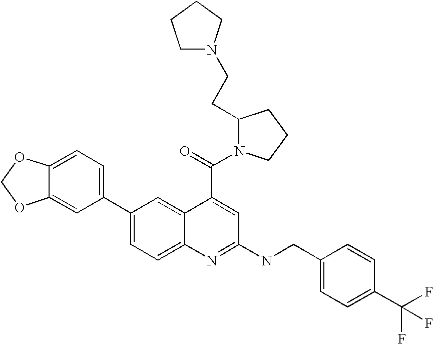 |
| 110 | 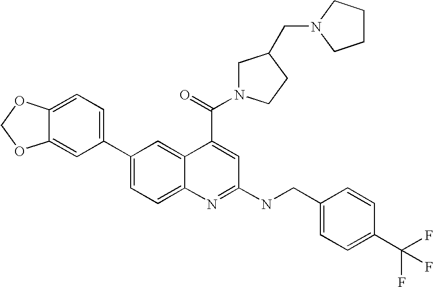 |
| 111 | 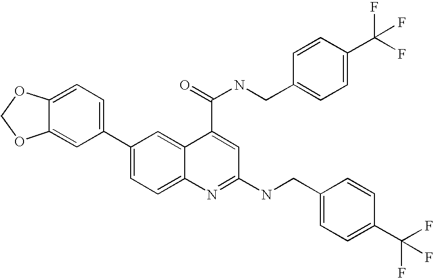 |
| 112 | 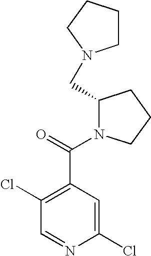 |
| 113 | 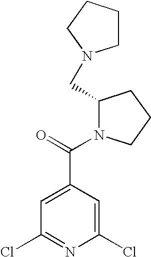 |
| 114 | 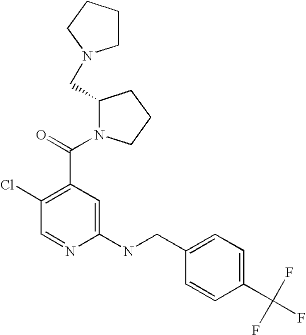 |
| 115 | 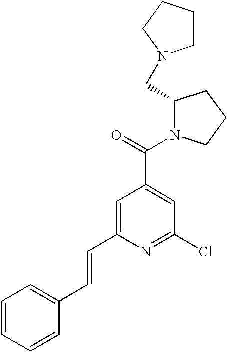 |
| 116 | 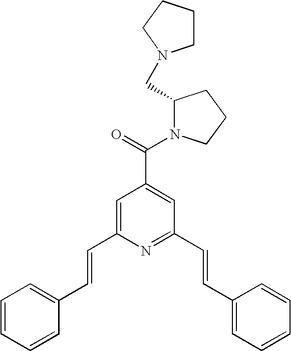 |
| 117 | 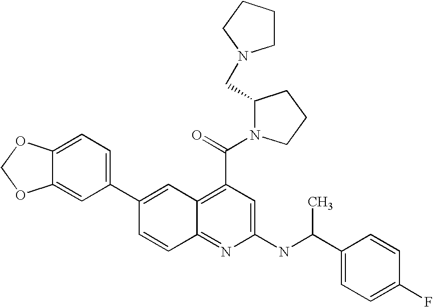 |
| 118 | 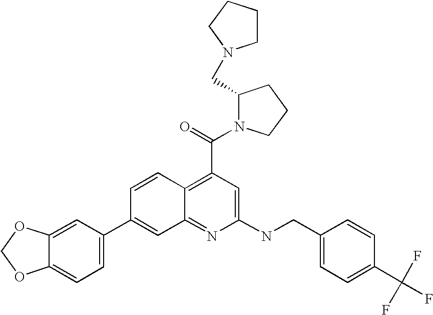 |
| 119 | 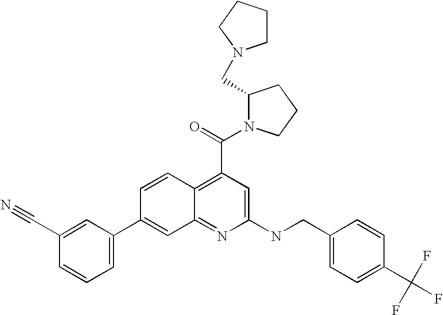 |
| 120 | 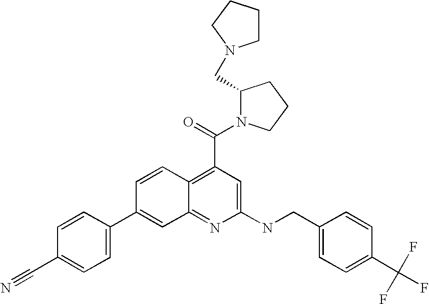 |
| 121 | 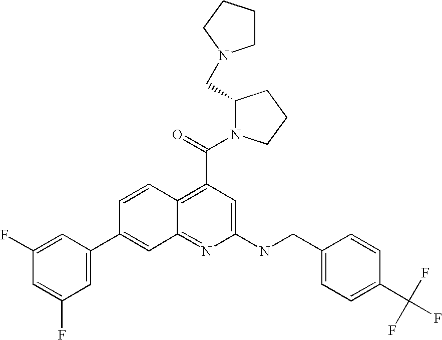 |
| 122 | 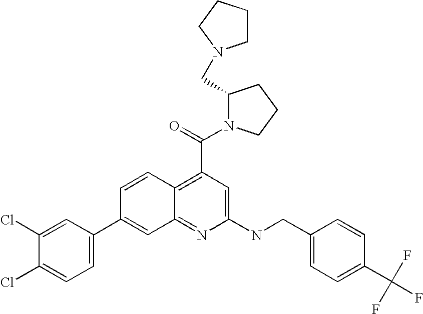 |
| 123 | 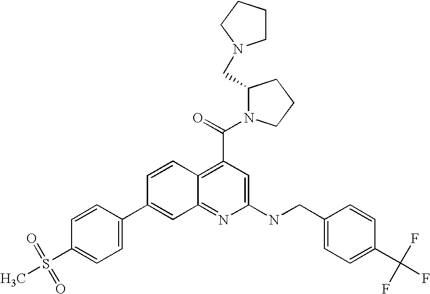 |
| 124 | 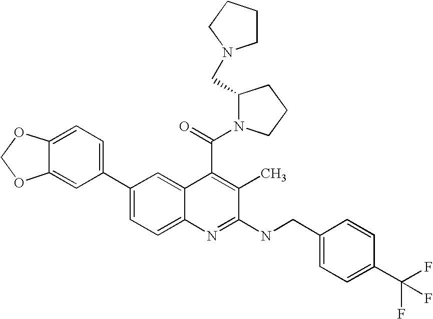 |
| 125 | 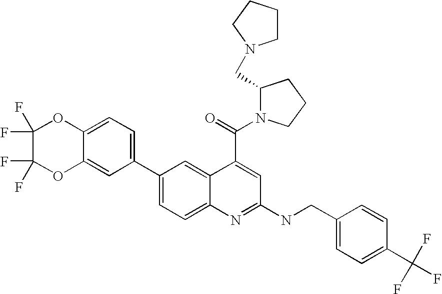 |
| 126 | 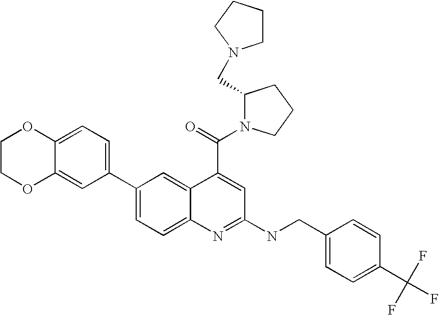 |
| 127 | 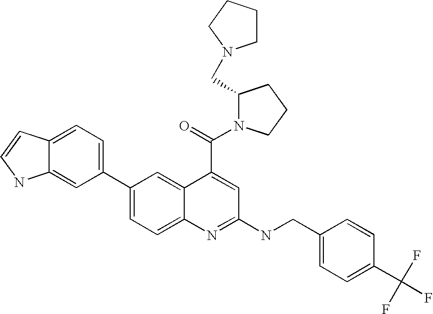 |
| 128 | 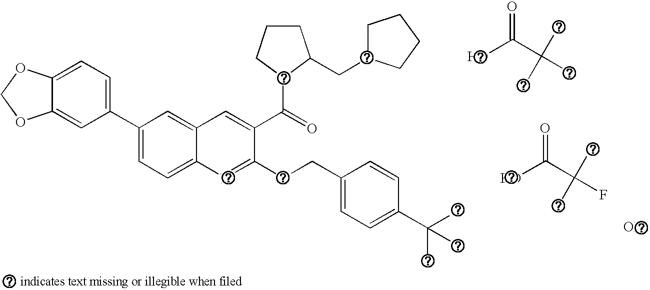 |
| 129 | 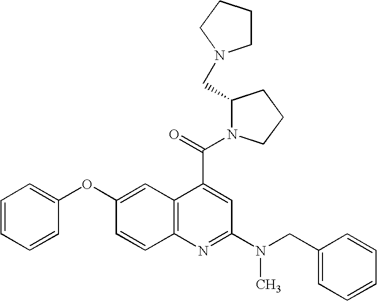 |
| 130 | 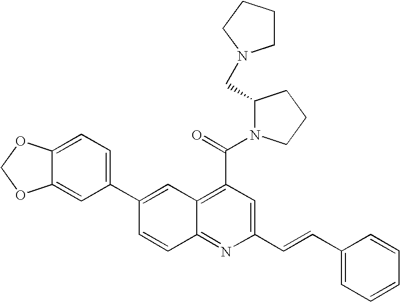 |
| 131 | 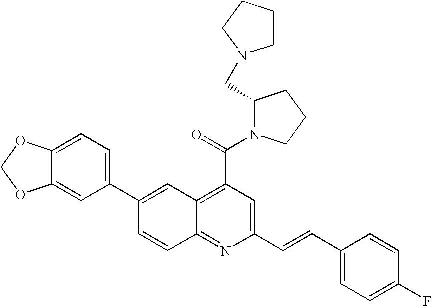 |
| 132 | 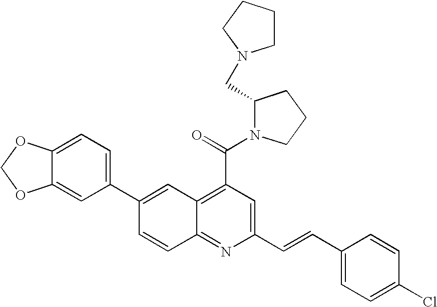 |
| 133 | 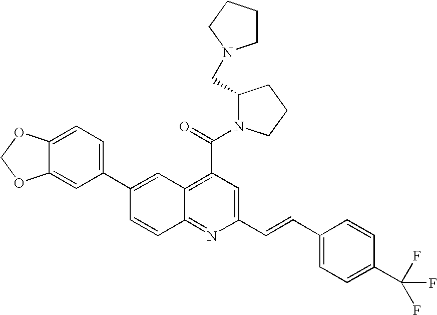 |
| 134 | 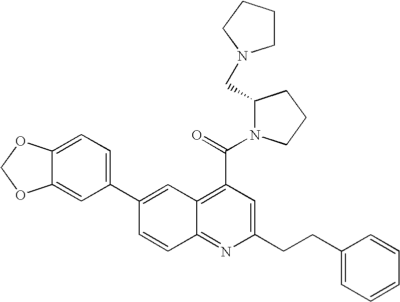 |
| 135 | 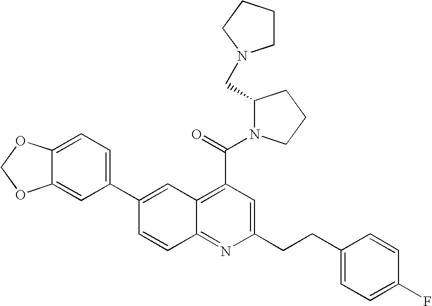 |
| 136 | 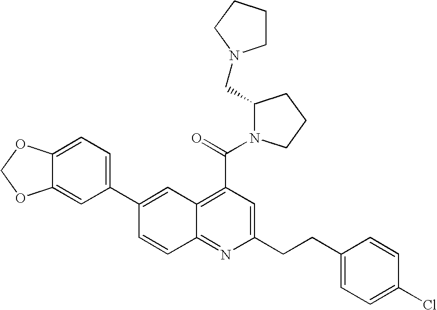 |
| 137 | 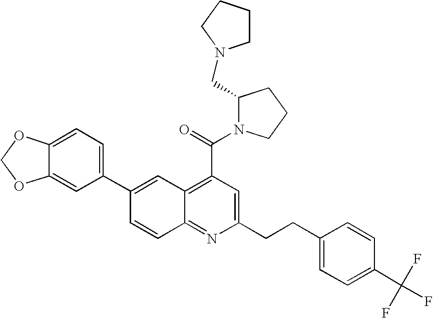 |
| 138 | 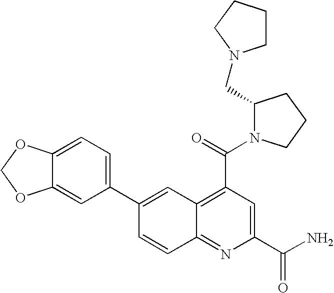 |
| 139 | 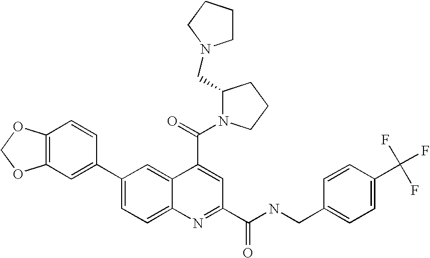 |
| 140 | 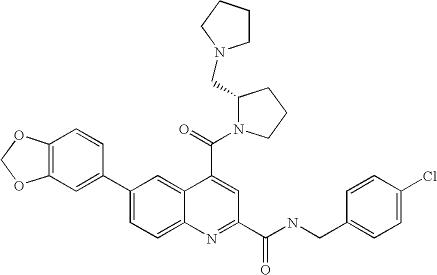 |
| 141 | 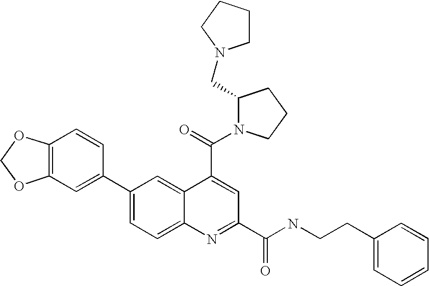 |
| 142 | 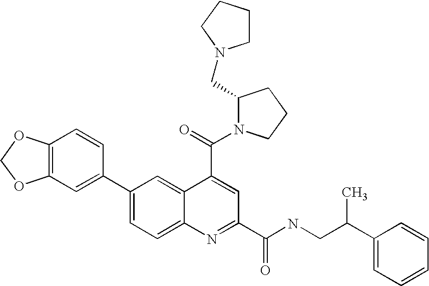 |
| 143 | 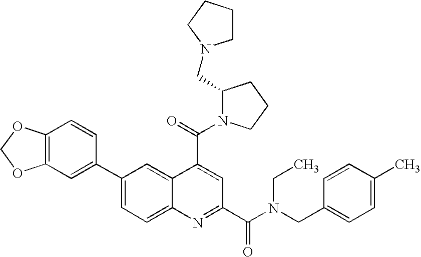 |
| 144 | 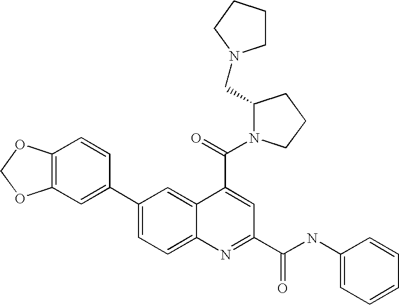 |
| 145 | 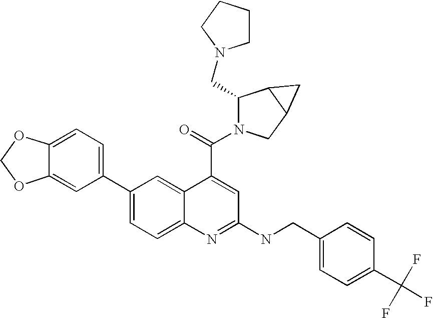 |
| 146 | 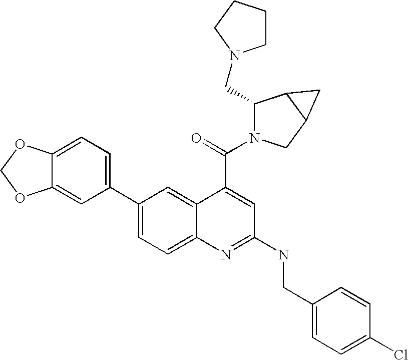 |
| 147 | 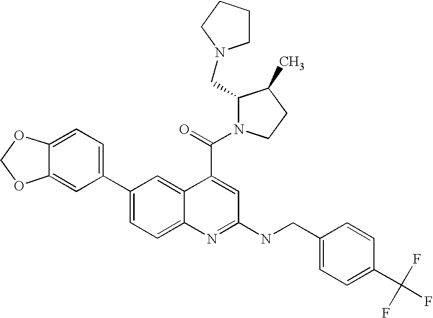 |
| 148 | 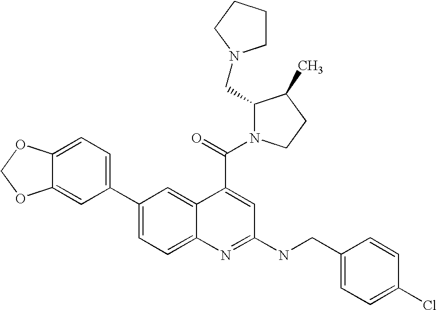 |
| 149 | 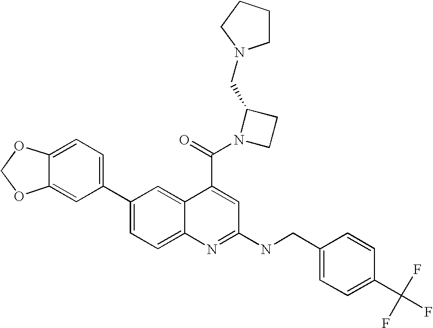 |
| 150 | 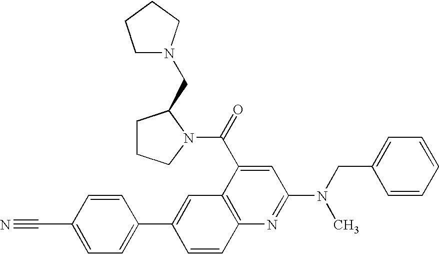 |
| 151 | 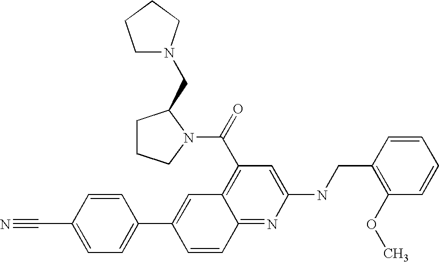 |
| 152 | 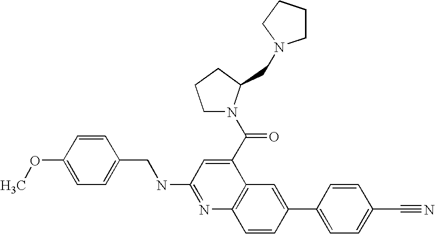 |
| 153 | 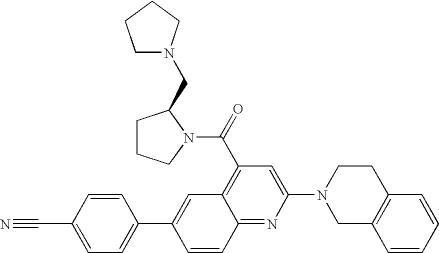 |
| 154 | 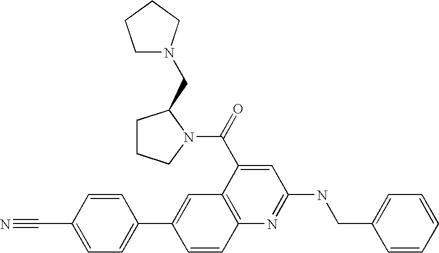 |
| 155 | 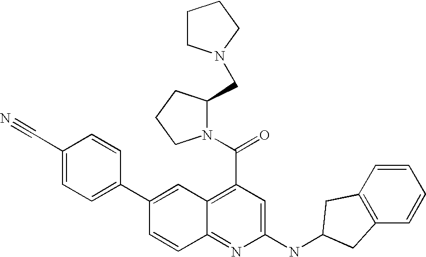 |
| 156 | 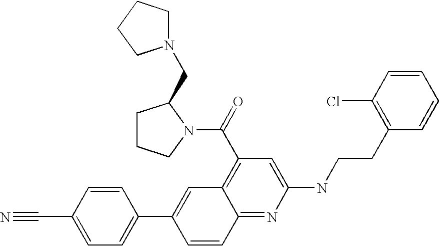 |
| 157 | 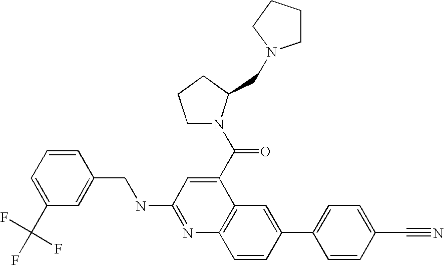 |
| 158 | 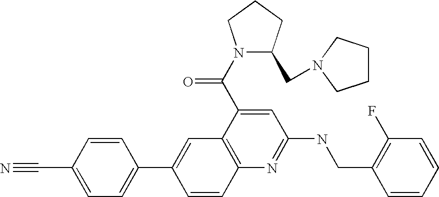 |
| 159 | 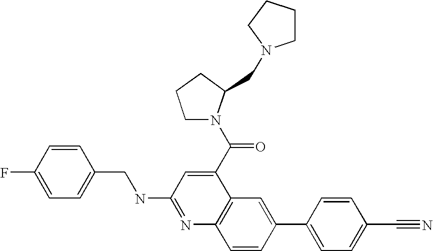 |
| 160 | 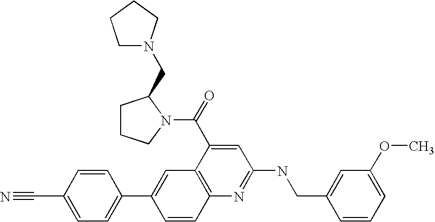 |
| 161 | 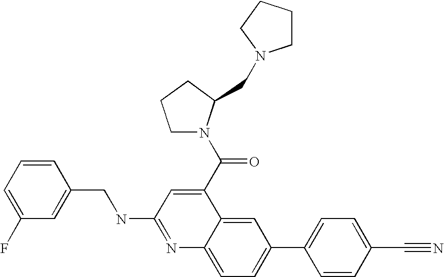 |
| 162 | 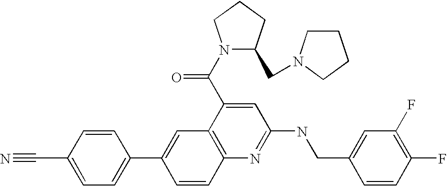 |
| 163 | 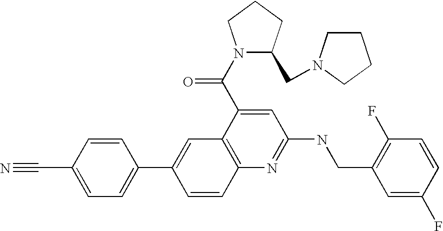 |
| 164 | 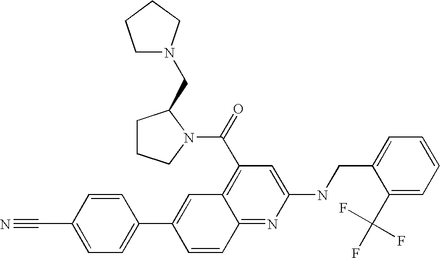 |
| 165 | 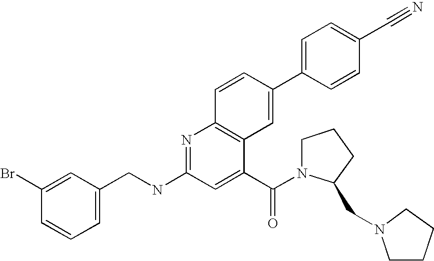 |
| 166 | 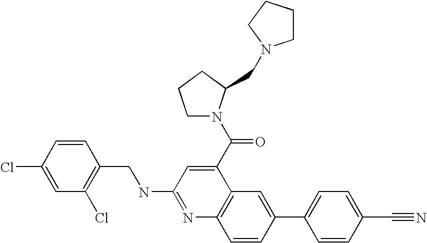 |
| 167 | 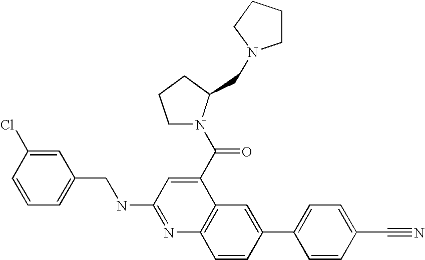 |
| 168 | 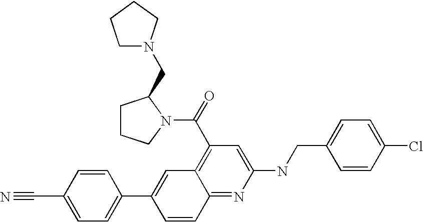 |
| 169 | 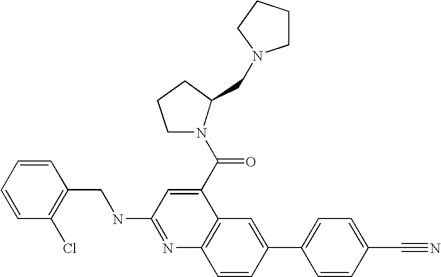 |
| 170 | 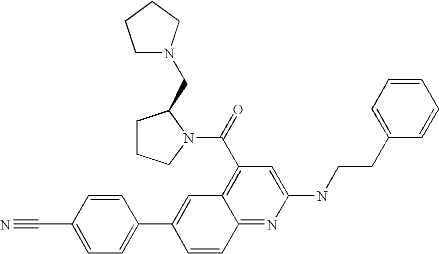 |
| 171 | 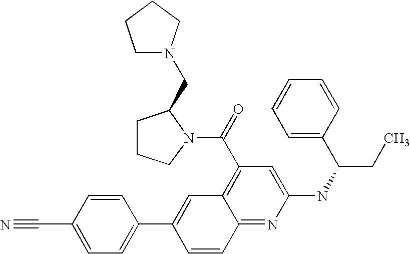 |
| 172 | 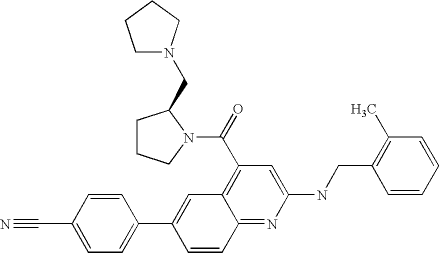 |
| 173 | 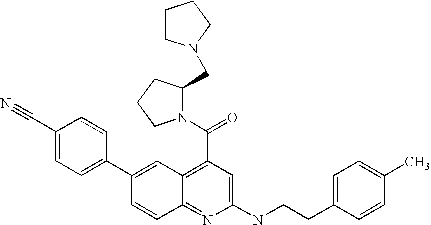 |
| 174 | 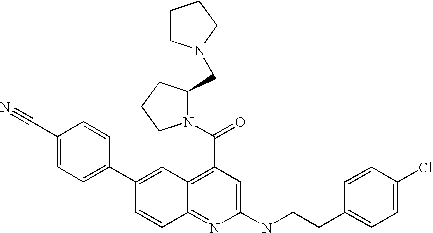 |
| 175 | 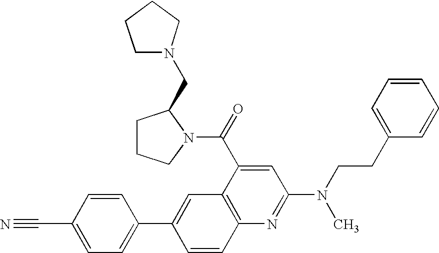 |
| 176 | 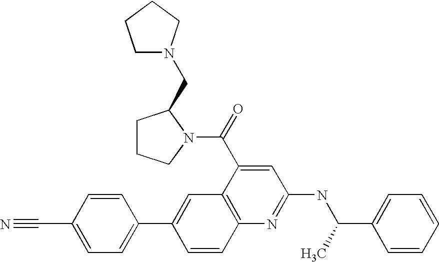 |
| 177 | 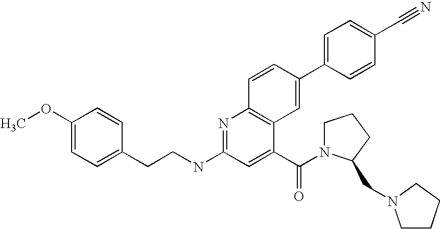 |
| 178 | 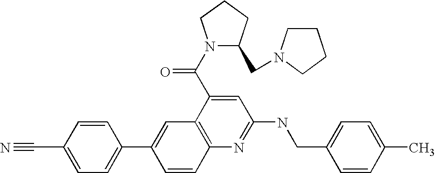 |
| 179 | 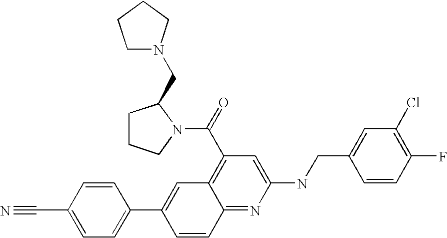 |
| 180 | 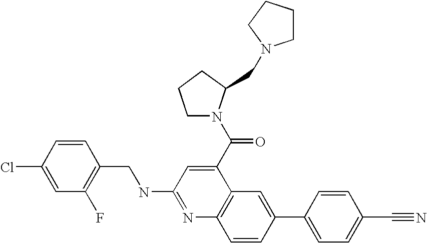 |
| 181 | 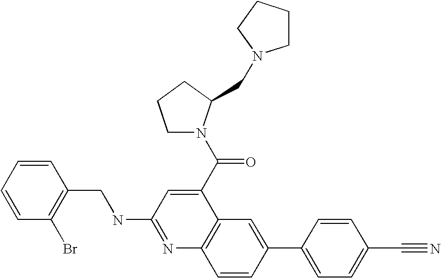 |
| 182 | 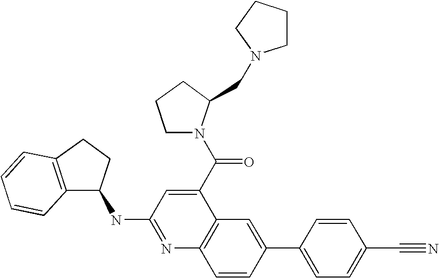 |
| 183 | 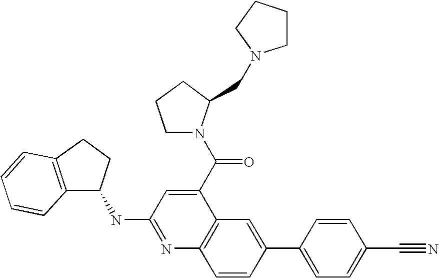 |
| 184 | 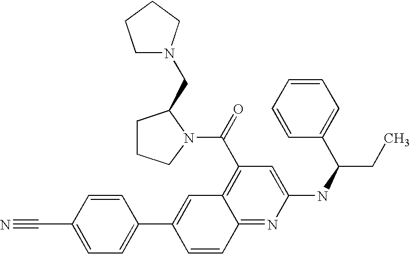 |
| 185 | 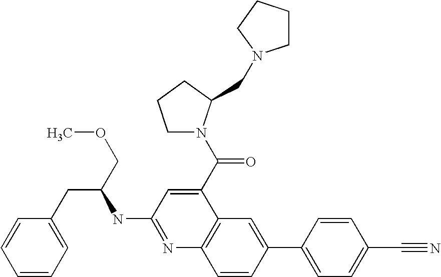 |
| 186 | 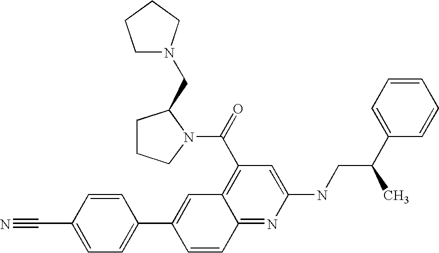 |
| 187 | 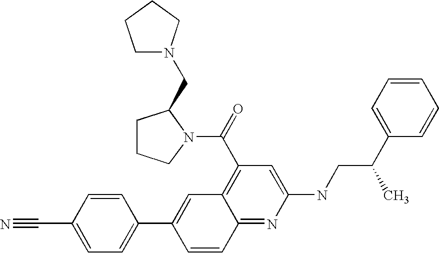 |
| 188 | 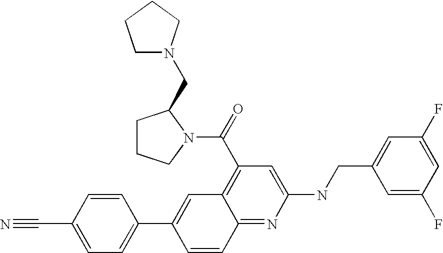 |
| 189 | 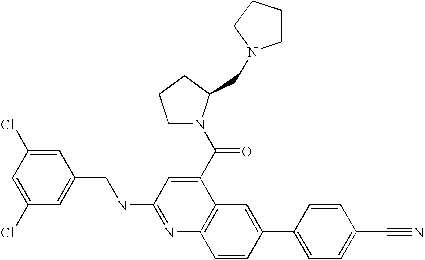 |
| 190 | 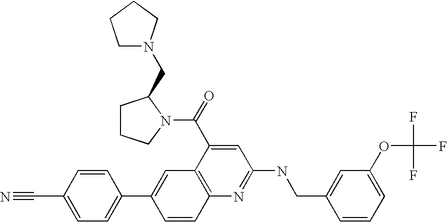 |
| 191 | 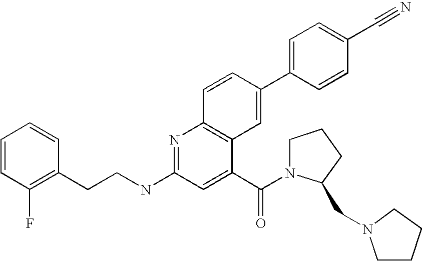 |
| 192 | 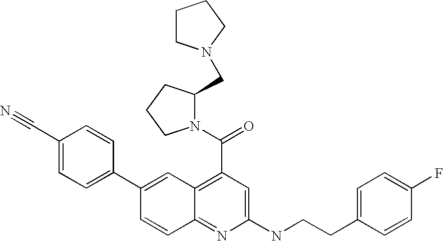 |
| 193 | 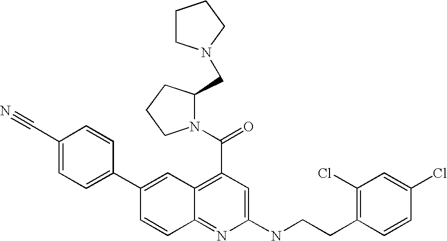 |
| 194 | 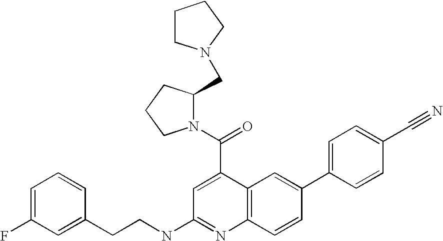 |
| 195 | 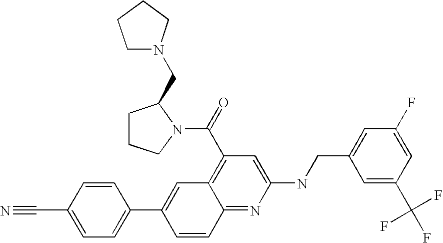 |
| 196 | 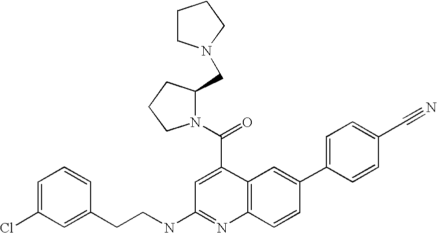 |
| 197 | 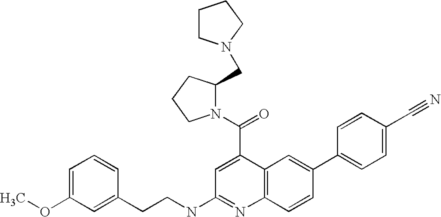 |
| 198 | 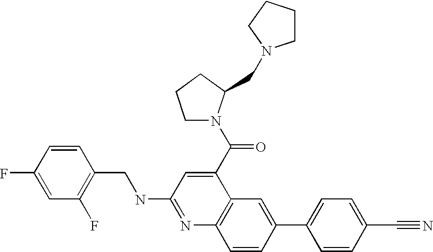 |
| 199 | 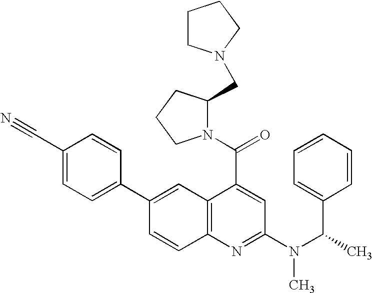 |
| 200 | 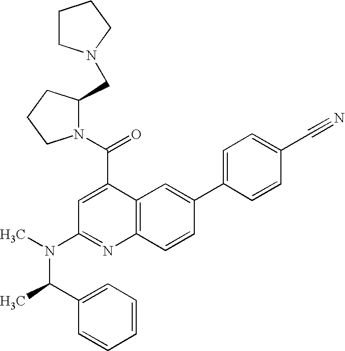 |
| 201 | 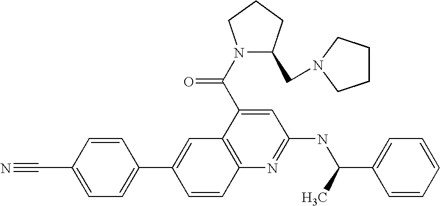 |
| 202 | 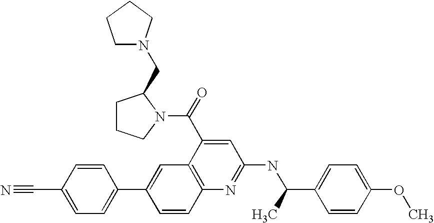 |
| 203 | 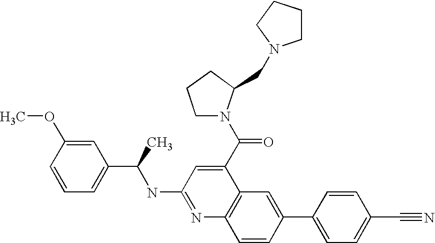 |
| 204 | 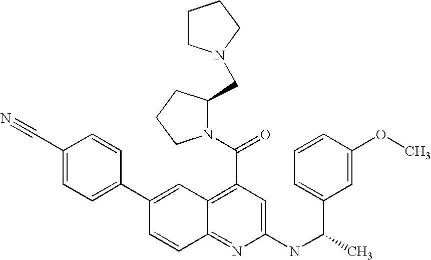 |
| 205 | 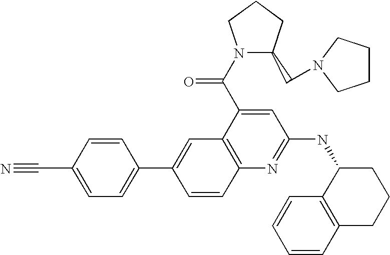 |
| 206 | 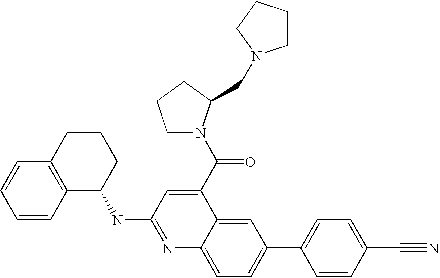 |
| 207 | 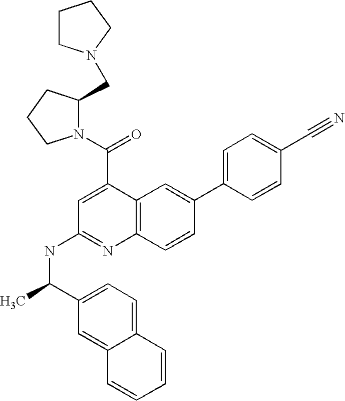 |
| 208 | 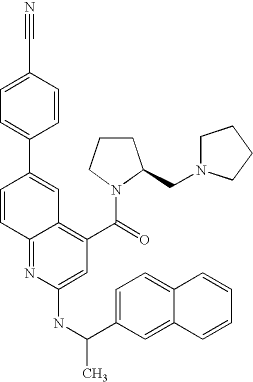 |
| 209 | 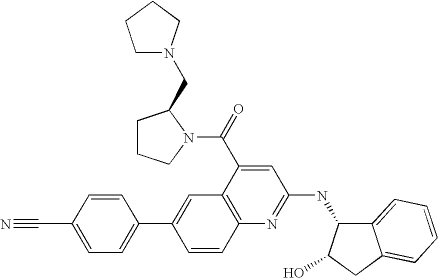 |
| 210 | 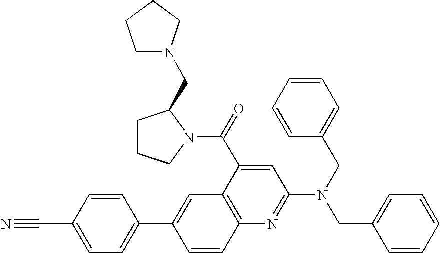 |
| 211 | 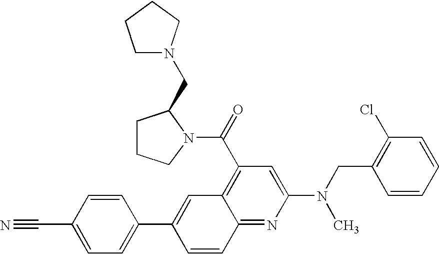 |
| 212 | 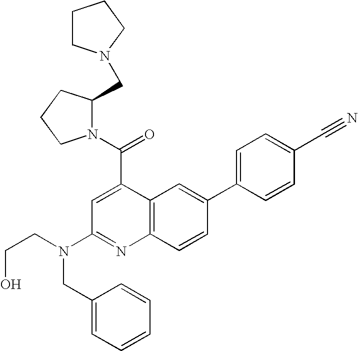 |
| 213 | 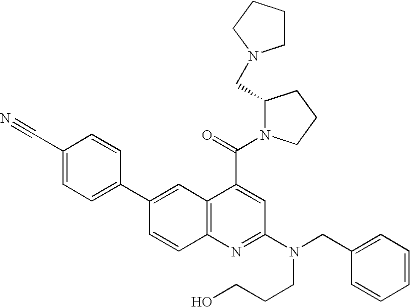 |
| 214 | 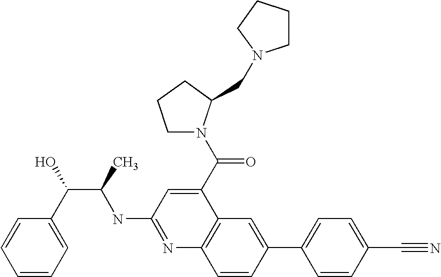 |
| 215 | 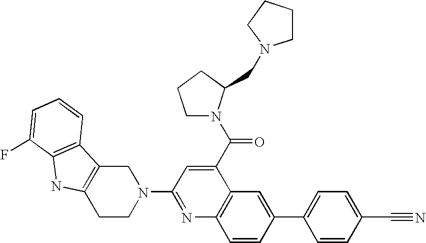 |
| 216 | 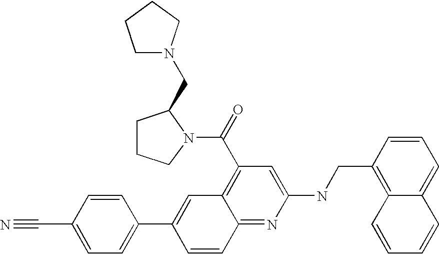 |
| 217 | 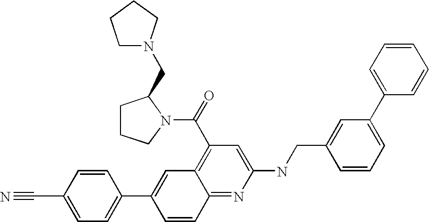 |
| 218 | 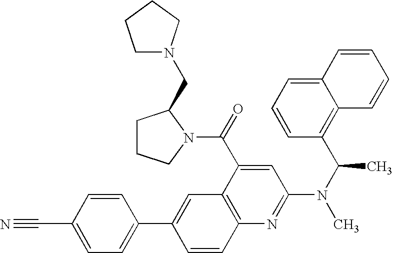 |
| 219 | 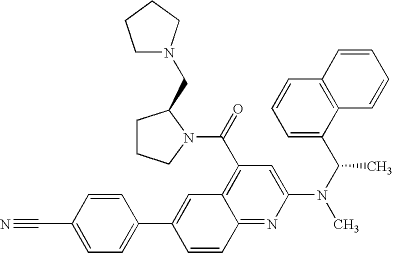 |
| 220 | 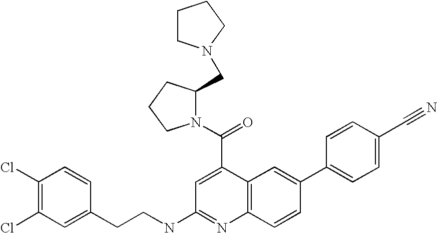 |
| 221 | 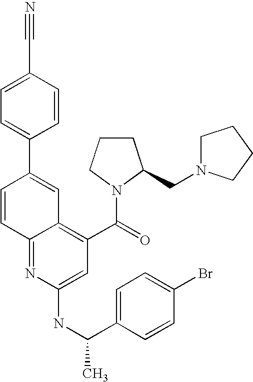 |
| 222 | 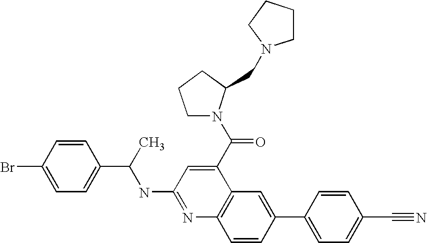 |
| 223 | 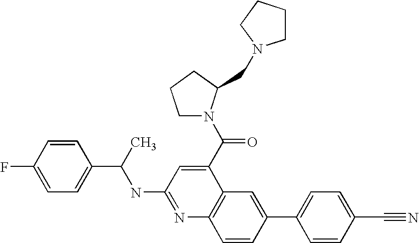 |
| 224 | 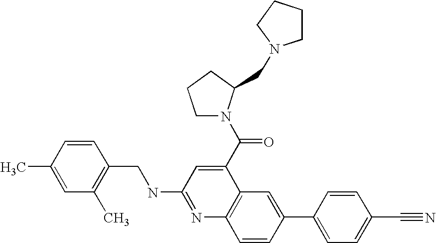 |
| 225 | 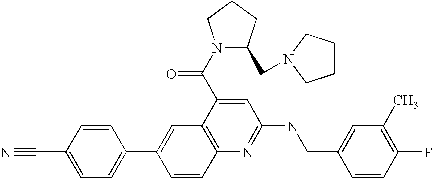 |
| 226 | 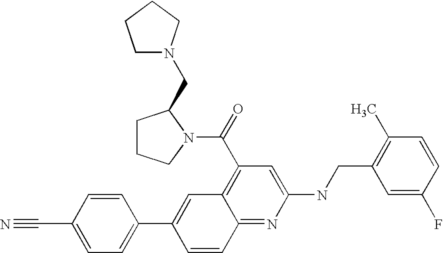 |
| 227 | 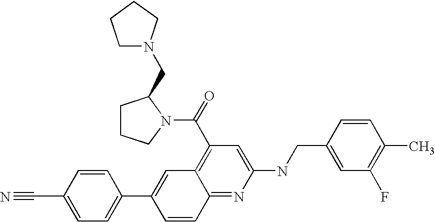 |
| 228 | 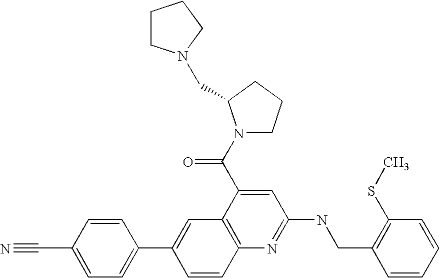 |
| 229 | 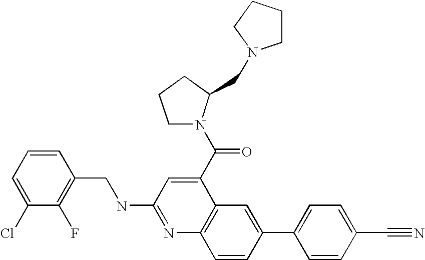 |
| 230 | 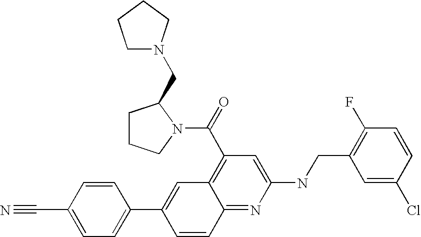 |
| 231 | 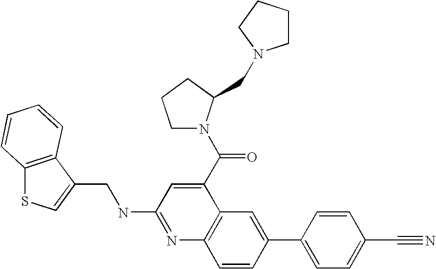 |
| 232 | 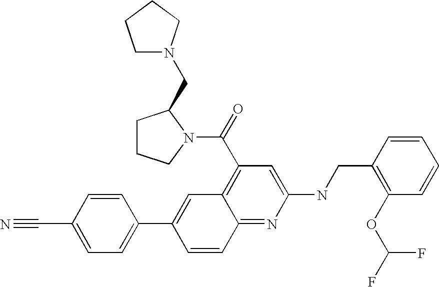 |
| 233 | 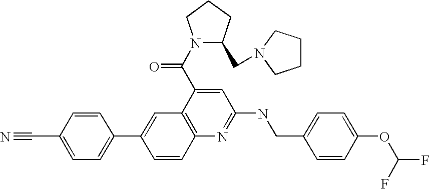 |
| 234 | 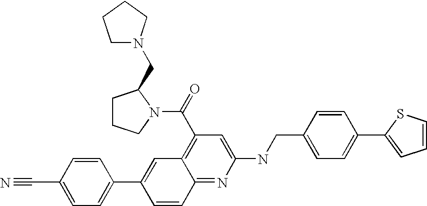 |
| 235 | 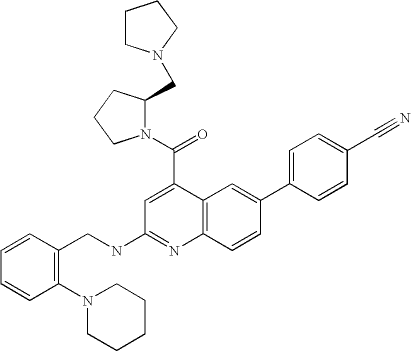 |
| 236 | 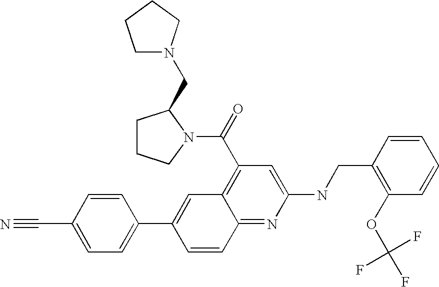 |
| 237 | 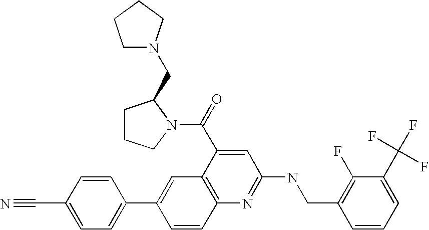 |
| 238 | 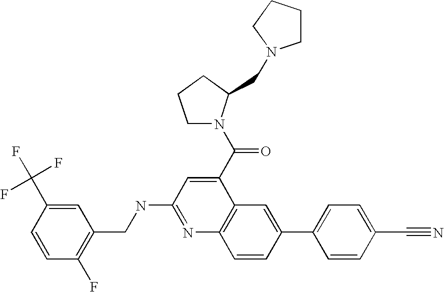 |
| 239 | 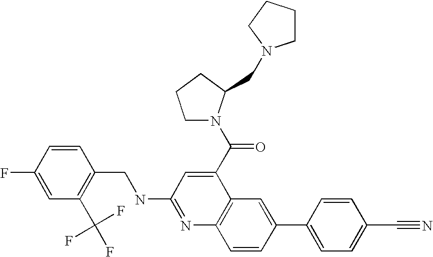 |
| 240 | 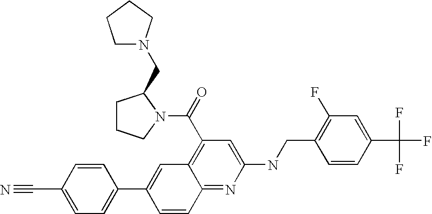 |
| 241 | 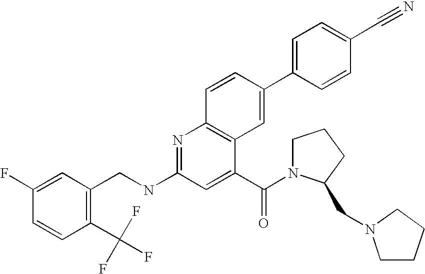 |
| 242 | 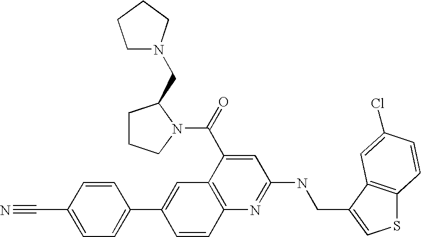 |
| 243 | 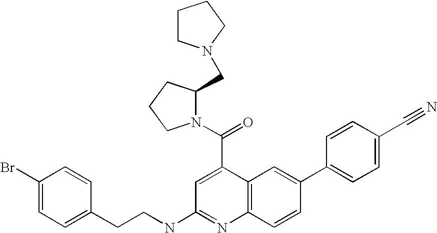 |
| 244 | 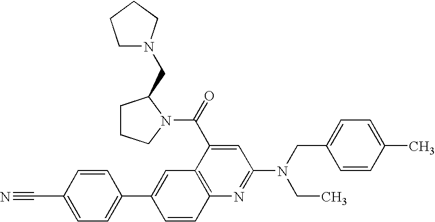 |
| 245 | 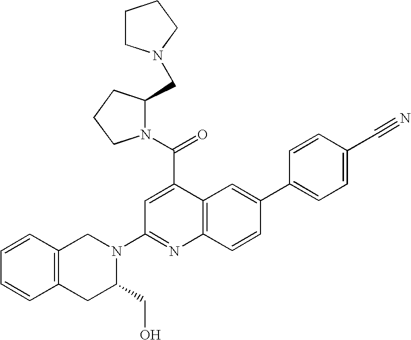 |
| 246 | 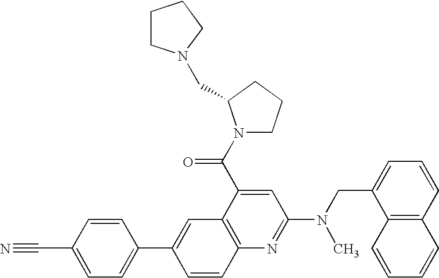 |
| 247 | 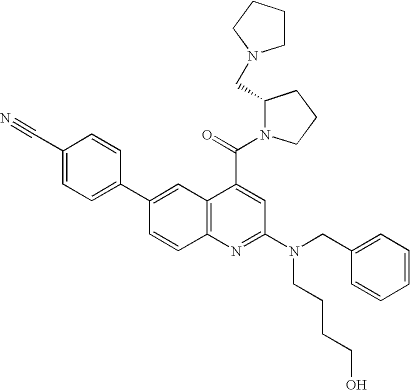 |
| 248 | 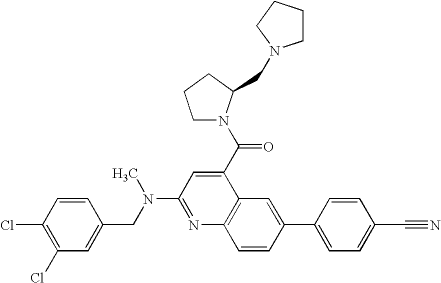 |
| 249 | 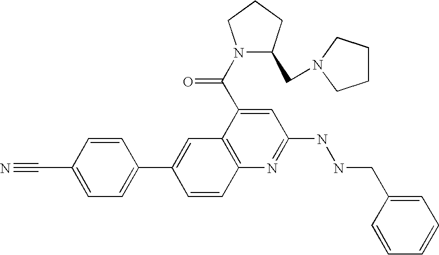 |
| 250 | 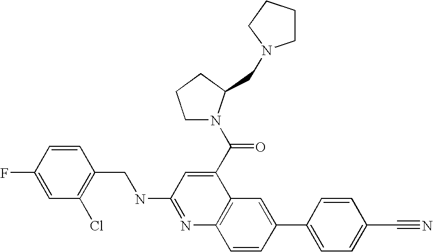 |
| 251 | 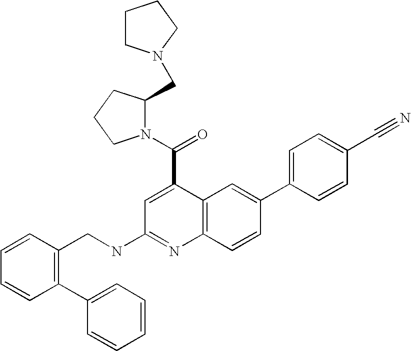 |
| 252 | 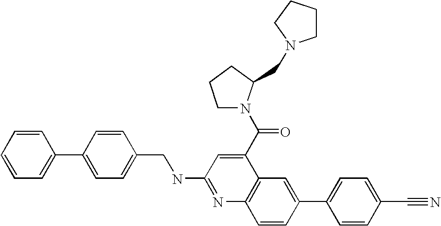 |
| 253 | 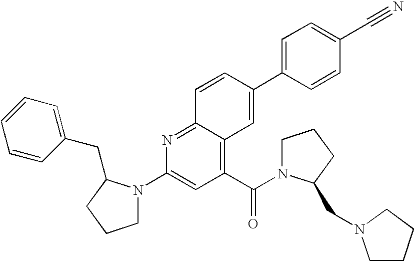 |
| 254 | 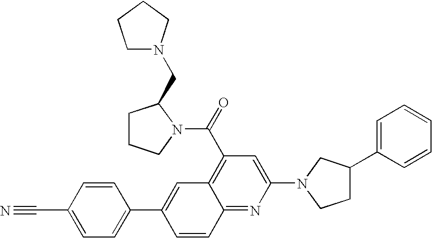 |
| 255 | 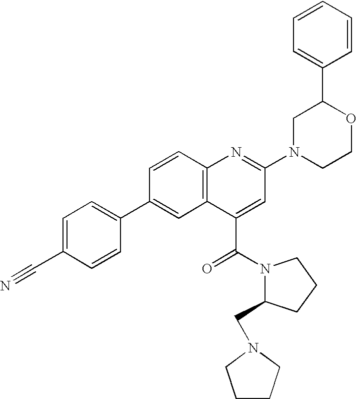 |
| 256 | 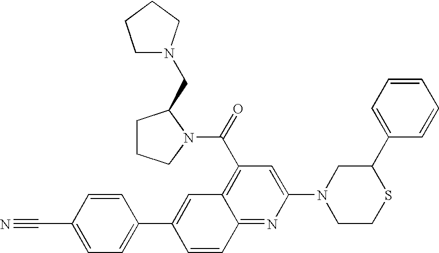 |
| 257 | 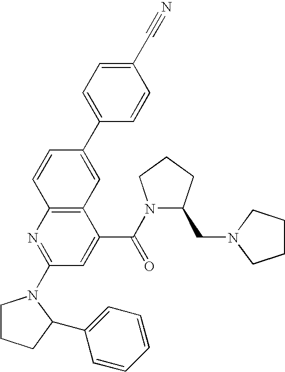 |
| 258 | 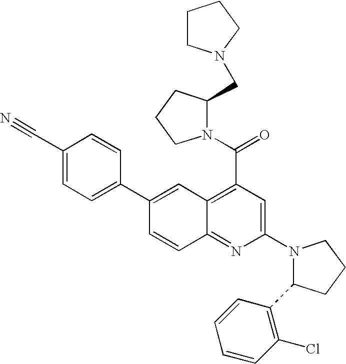 |
| 259 | 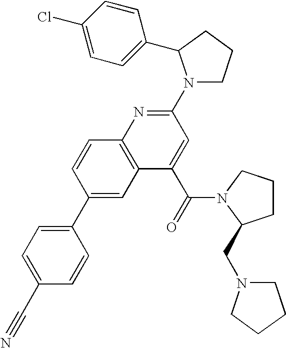 |
| 260 | 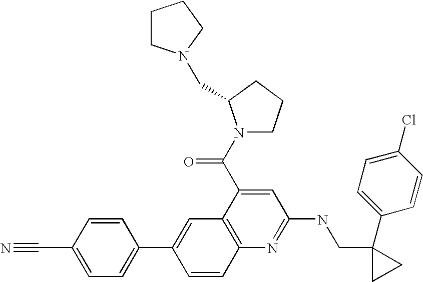 |
| 261 | 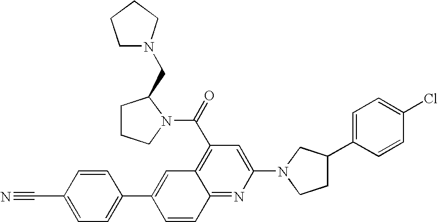 |
| 262 | 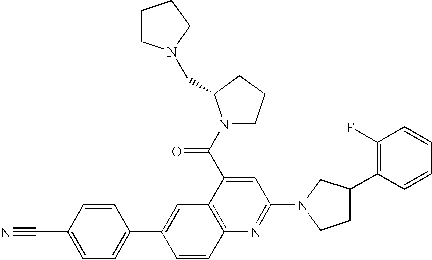 |
| 263 | 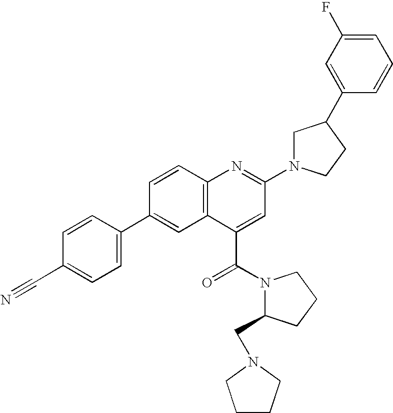 |
| 264 | 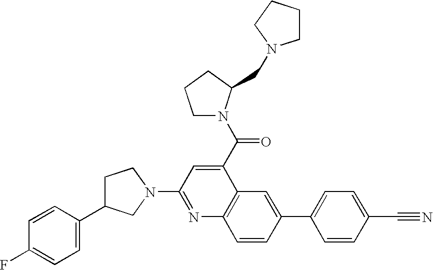 |
| 265 | 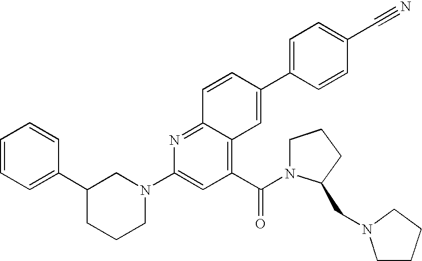 |
| 266 | 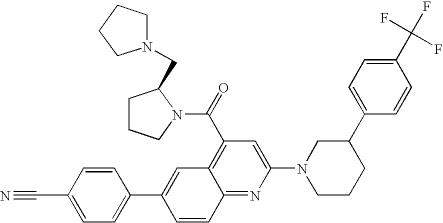 |
| 267 | 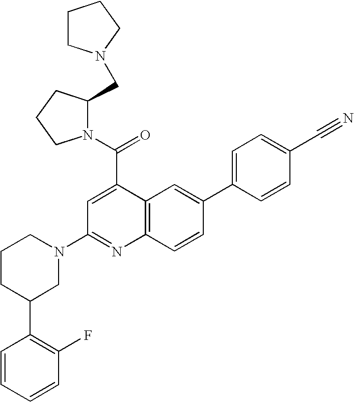 |
| 268 | 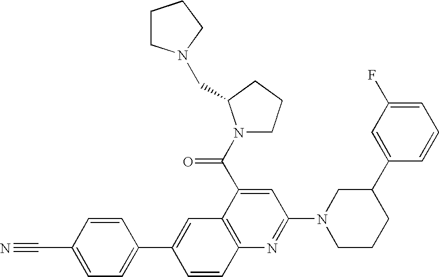 |
| 269 | 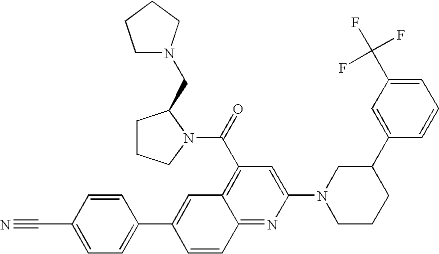 |
| 270 | 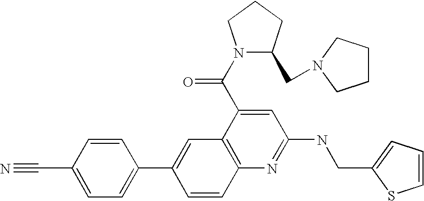 |
| 271 | 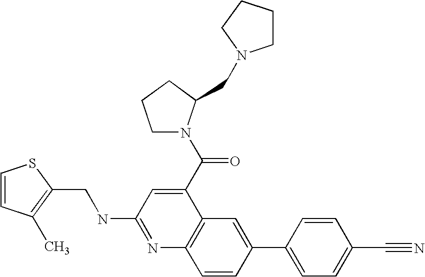 |
| 272 | 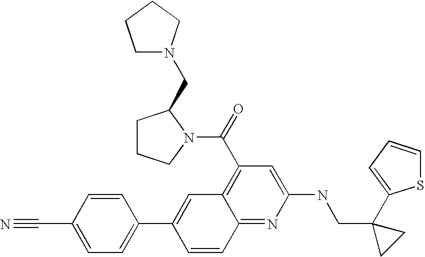 |
| 273 | 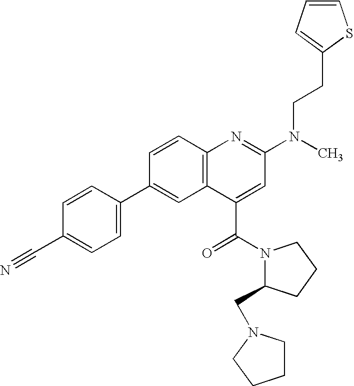 |
| 274 | 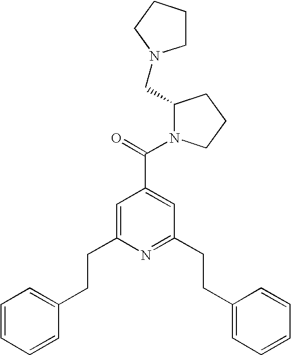 |
| 275 | 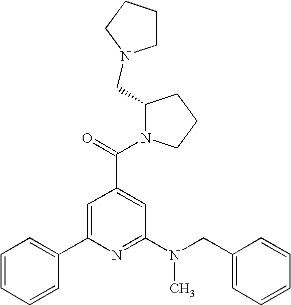 |
| 276 | 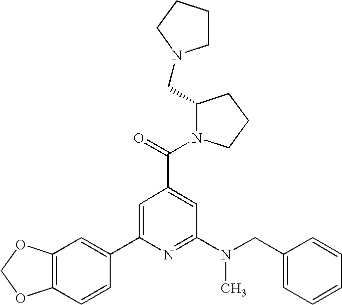 |
| 277 | 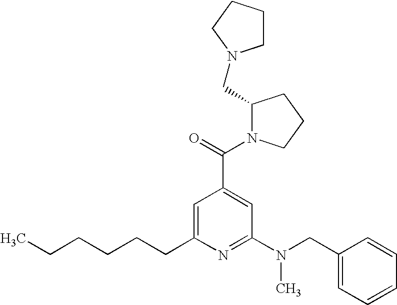 |
| 278 | 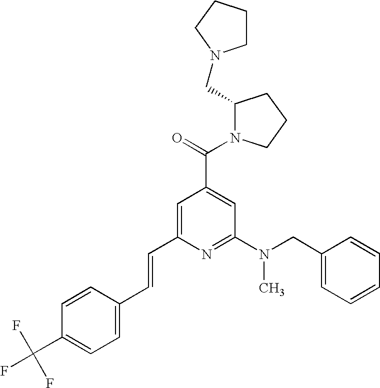 |
| 279 | 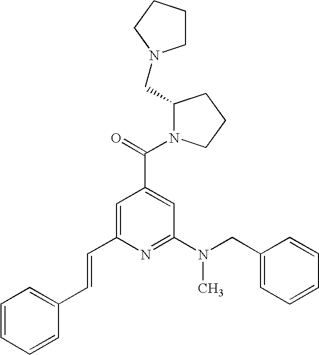 |
| 280 | 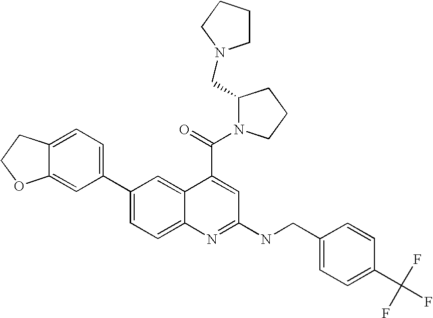 |
| 281 | 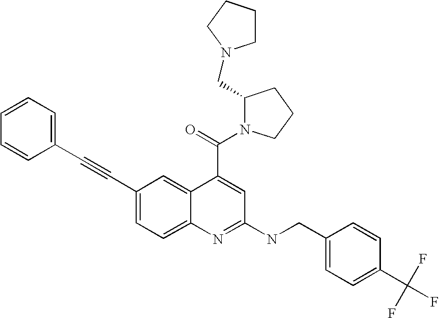 |
| 282 | 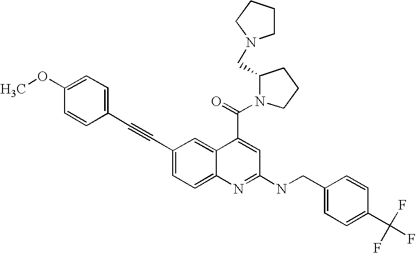 |
| 283 | 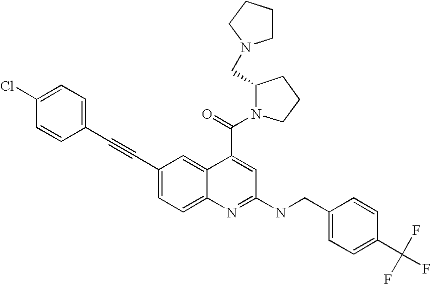 |
| 284 | 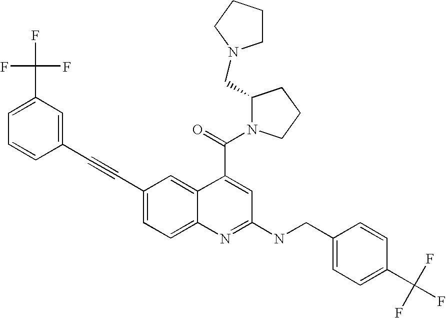 |
| 285 | 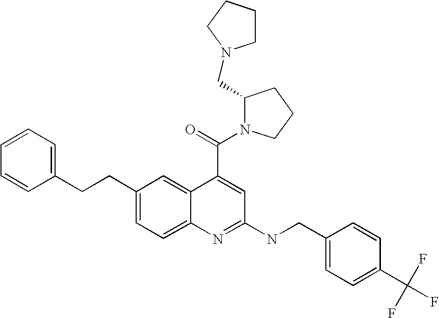 |
| 286 | 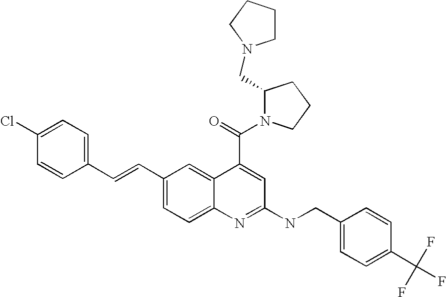 |
| 287 | 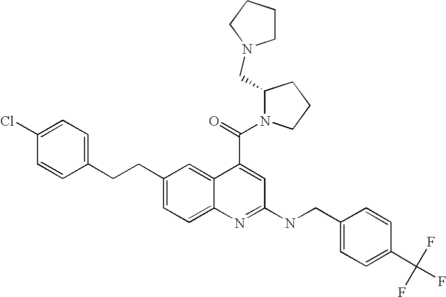 |
| 288 | 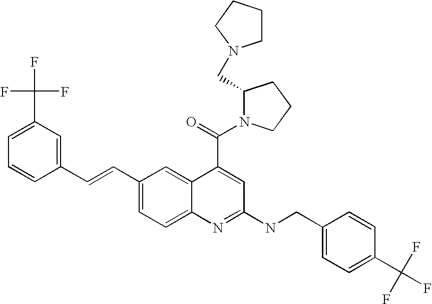 |
| 289 | 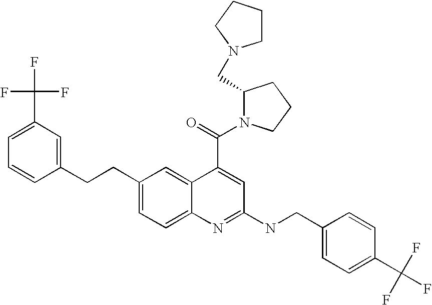 |
| 290 | 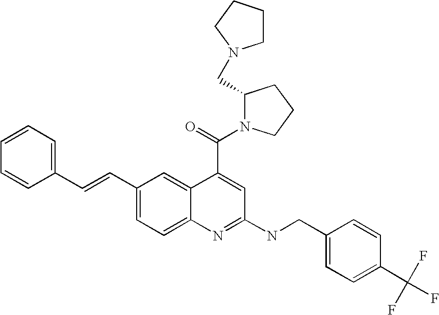 |
| 291 | 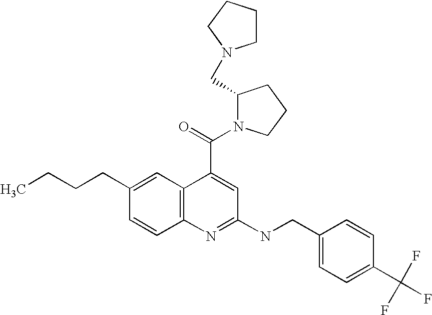 |
| 292 | 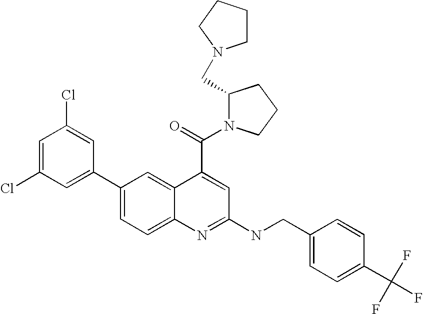 |
| 293 | 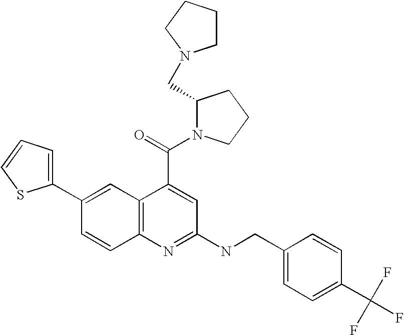 |
| 294 | 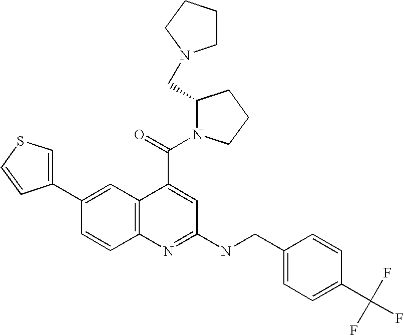 |
| 295 | 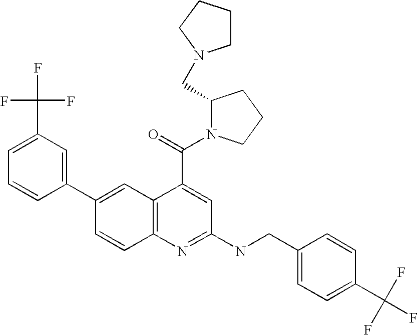 |
| 296 | 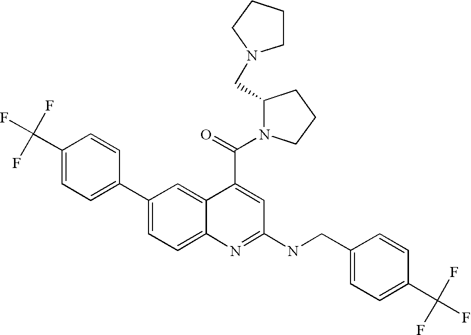 |
| 297 | 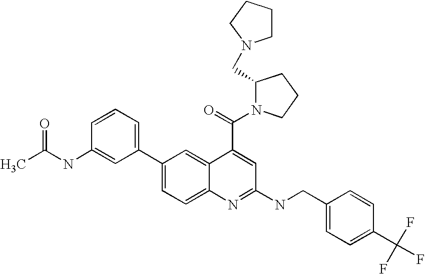 |
| 298 | 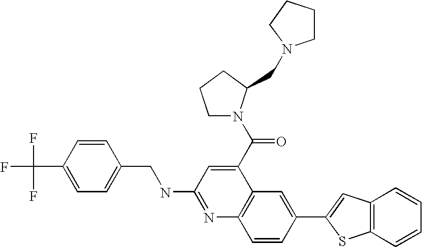 |
| 299 | 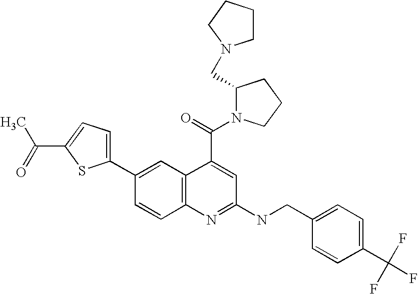 |
| 300 | 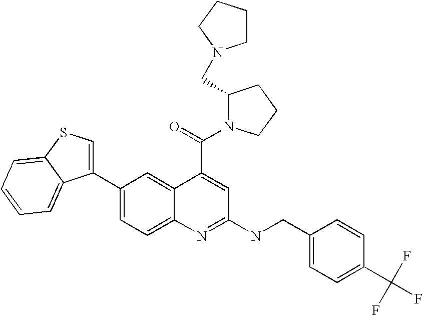 |
| 301 | 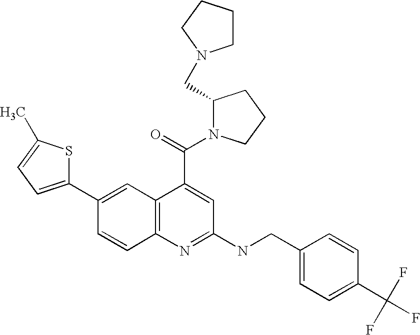 |
| 302 | 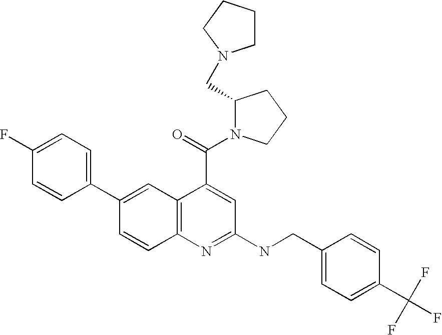 |
| 303 | 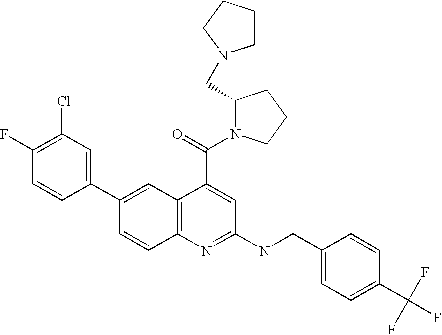 |
| 304 | 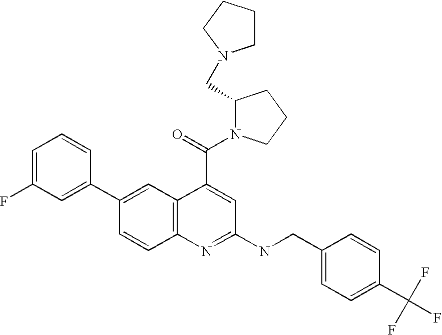 |
| 305 | 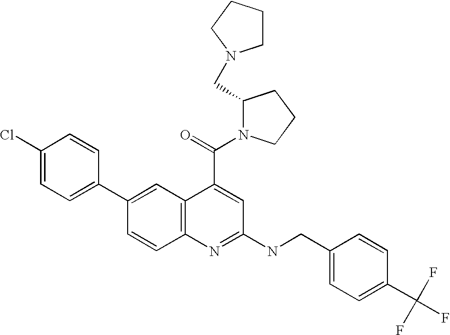 |
| 306 | 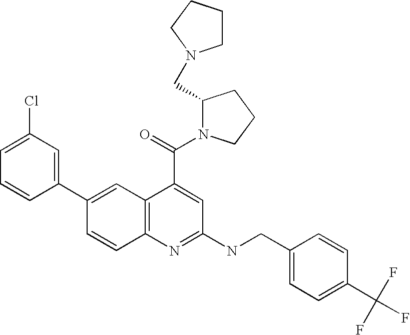 |
| 307 | 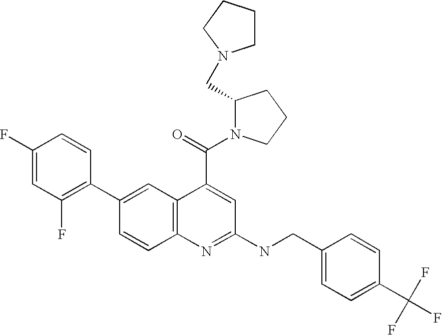 |
| 308 | 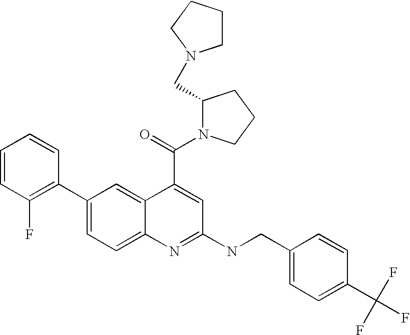 |
| 309 | 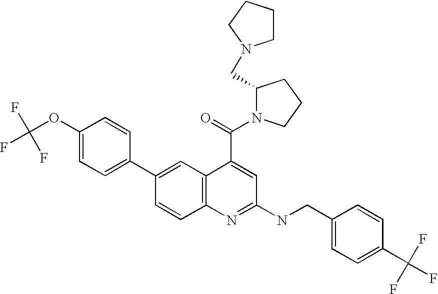 |
| 310 | 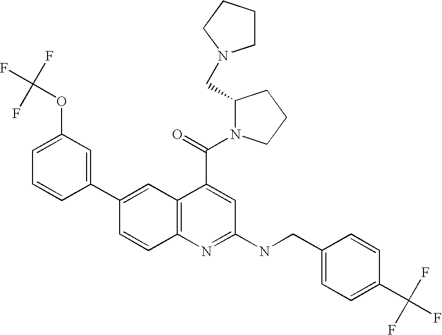 |
| 311 | 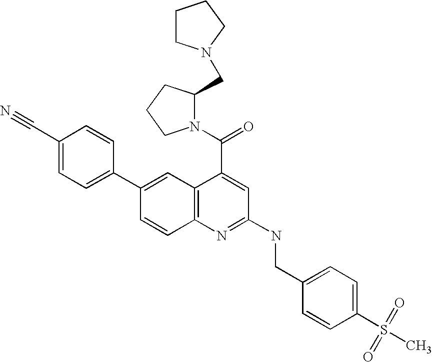 |
| 312 | 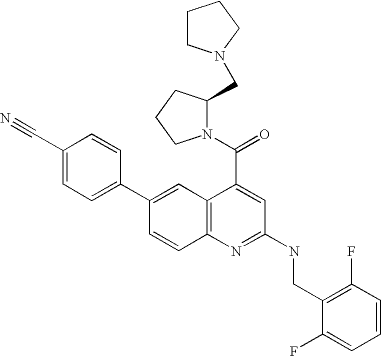 |
| 313 | 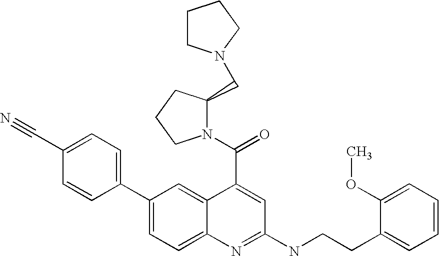 |
| 314 | 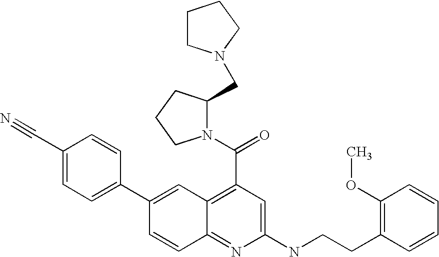 |
| 315 | 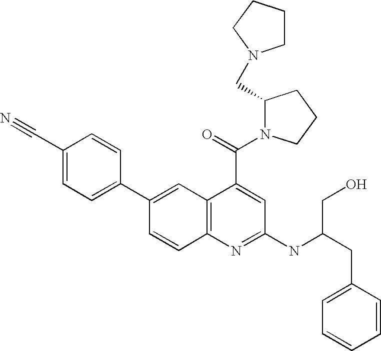 |
| 316 | 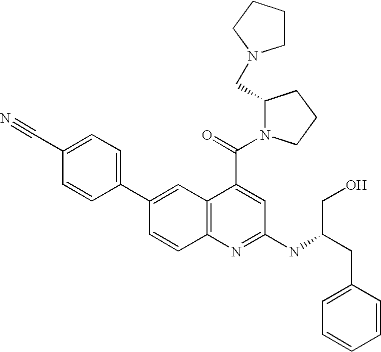 |
| 317 | 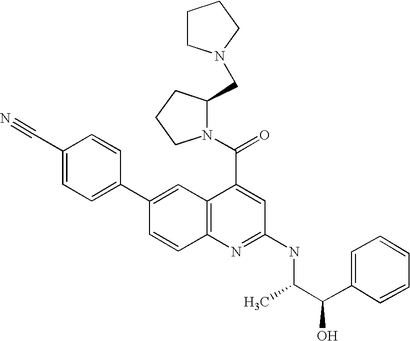 |
| 318 | 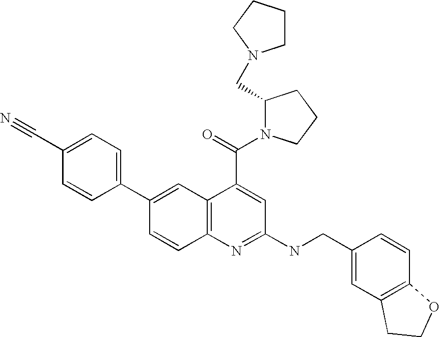 |
| 319 | 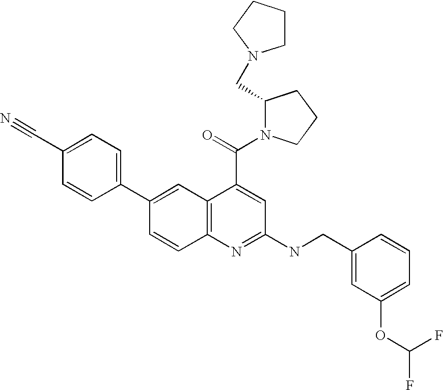 |
| 320 | 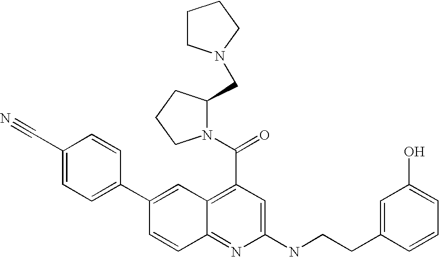 |
| 321 | 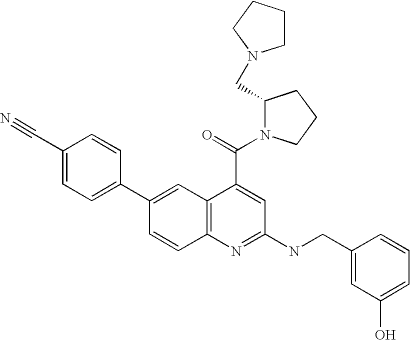 |
| 322 | 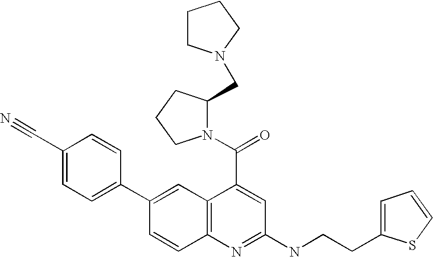 |
| 323 | 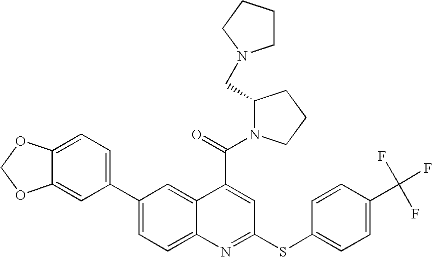 |
| 324 | 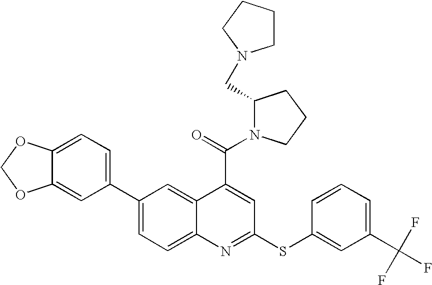 |
| 325 | 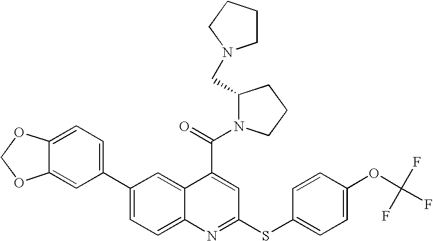 |
| 326 | 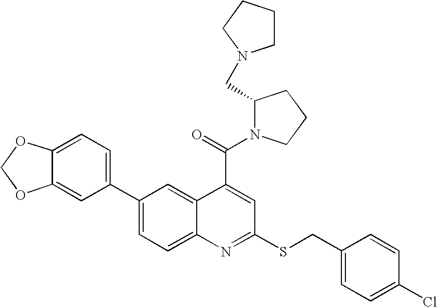 |
| 327 | 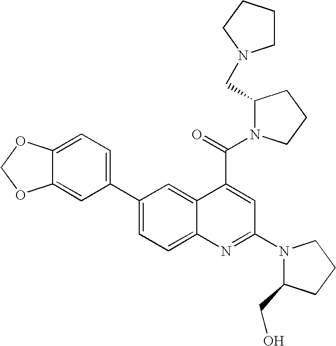 |
| 328 | 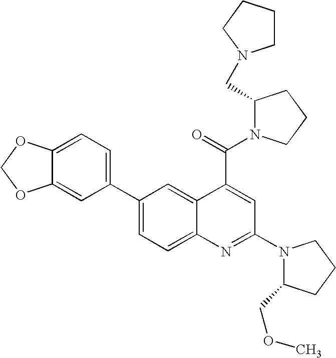 |
| 329 | 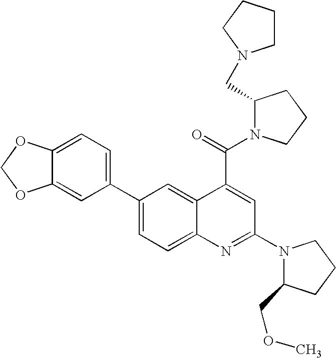 |
| 330 | 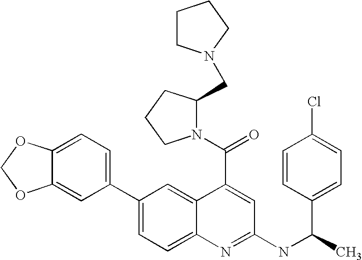 |
| 331 | 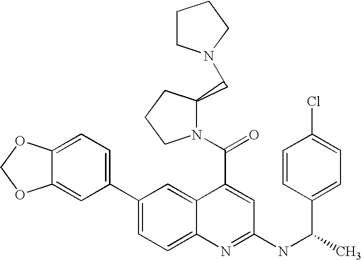 |
| 332 | 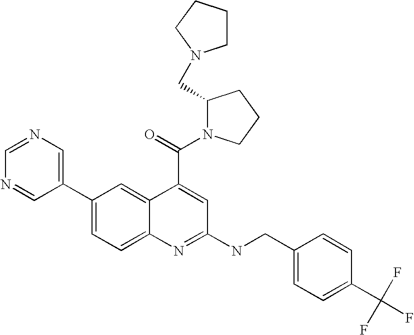 |
| 333 | 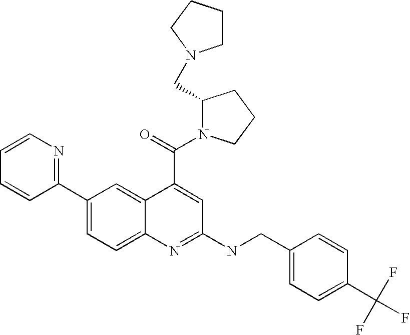 |
| 334 | 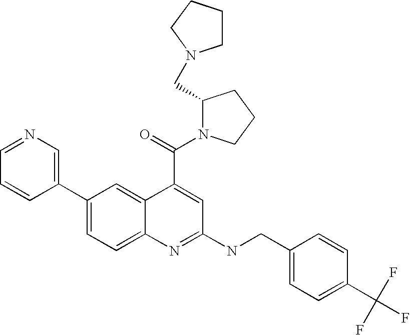 |
| 335 | 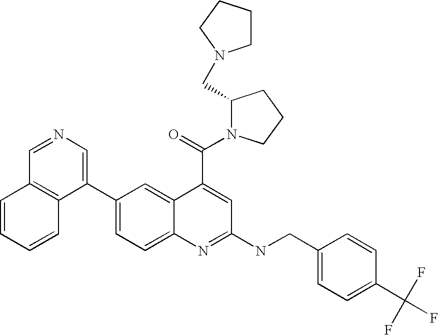 |
| 336 | 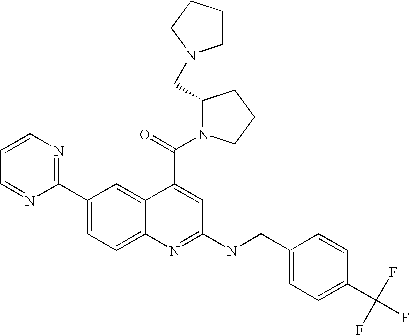 |
| 337 | 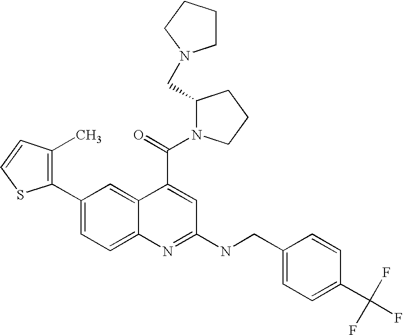 |
| 338 | 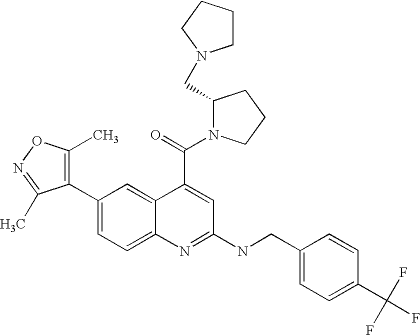 |
| 339 | 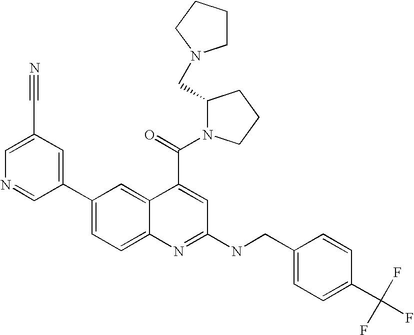 |
| 340 | 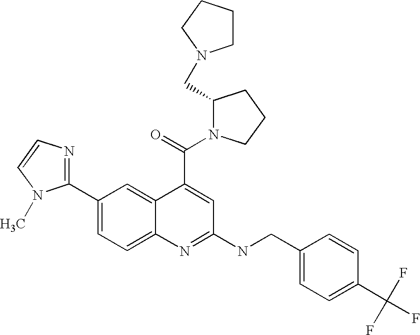 |
| 341 | 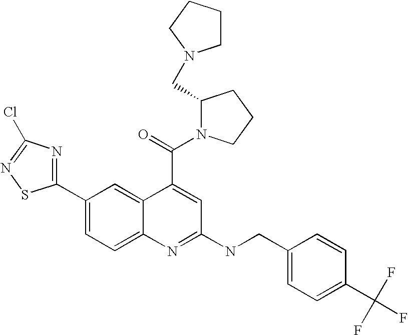 |
| 342 | 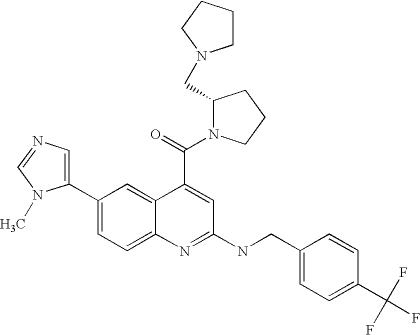 |
| 343 | 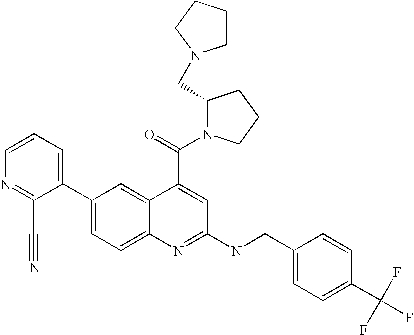 |
| 344 | 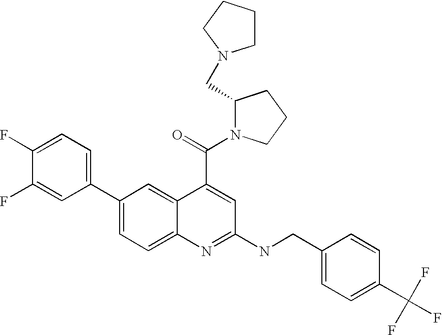 |
| 345 | 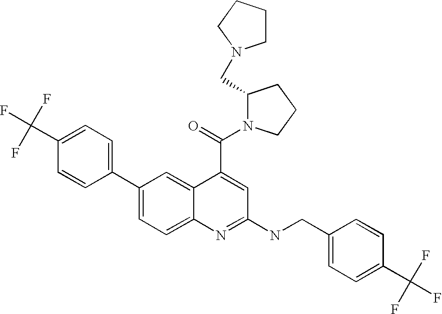 |
| 346 | 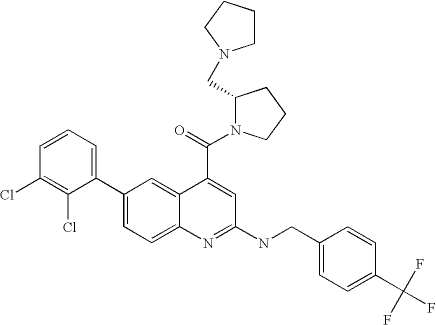 |
| 347 | 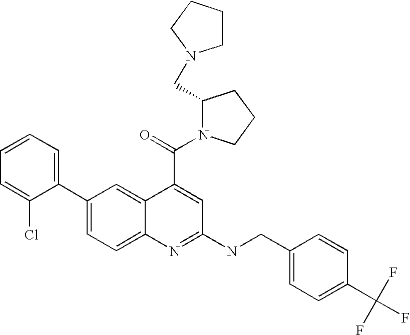 |
| 348 | 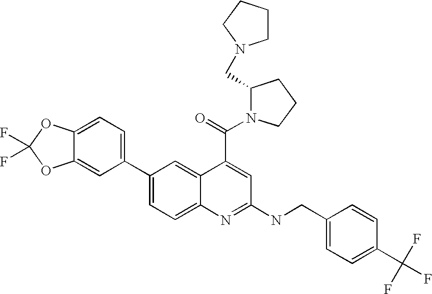 |
| 349 | 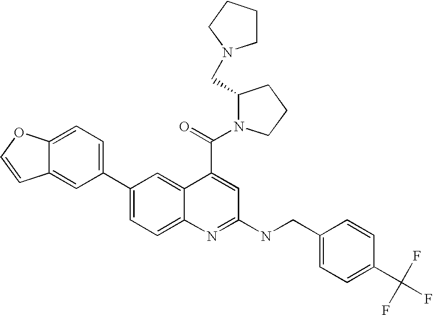 |
| 350 | 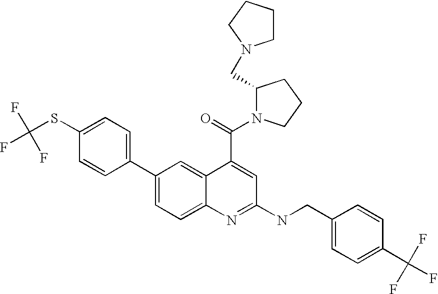 |
| 351 | 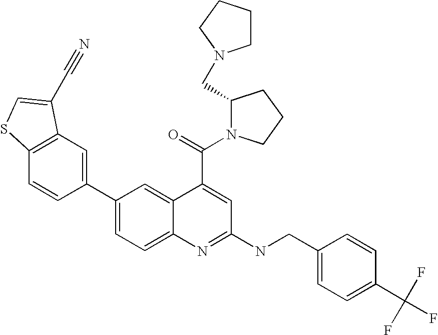 |
| 352 | 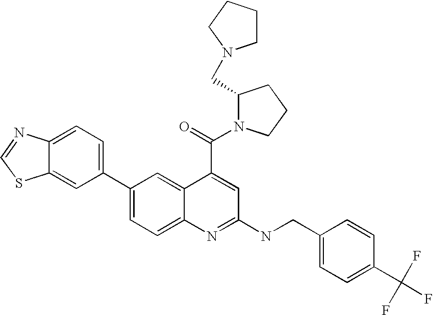 |
| 353 | 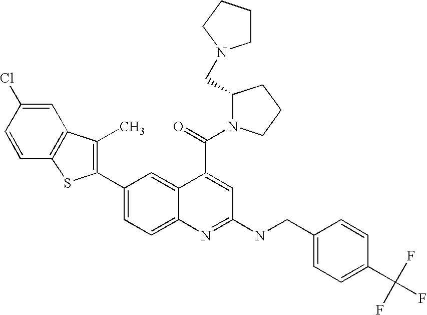 |
| 354 | 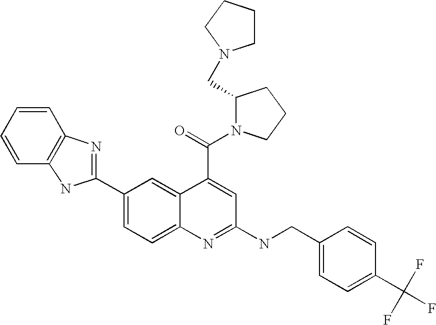 |
| 355 | 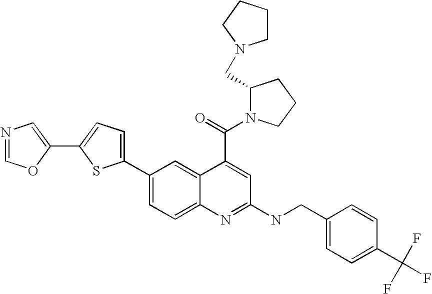 |
| 356 | 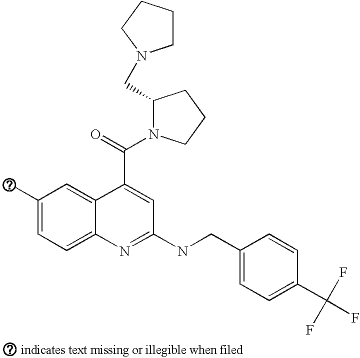 |
| 357 | 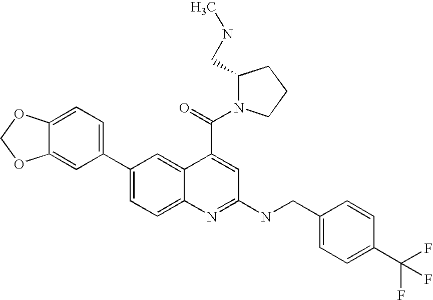 |
| 358 | 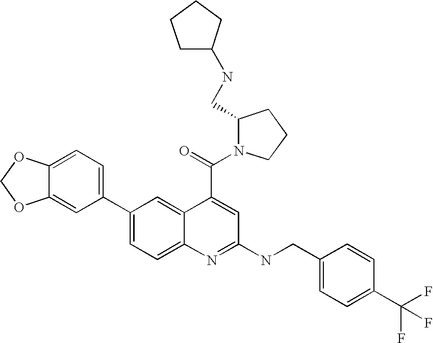 |
| 359 | 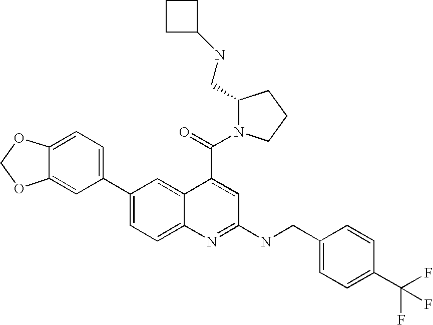 |
| 360 | 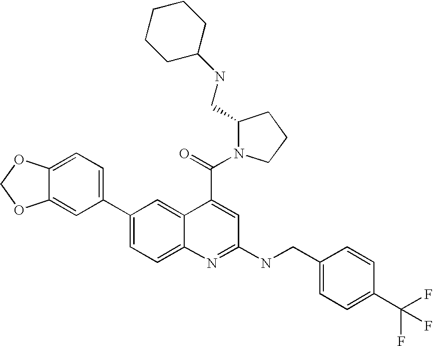 |
| 361 | 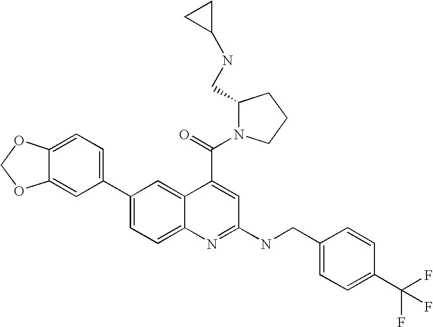 |
| 362 | 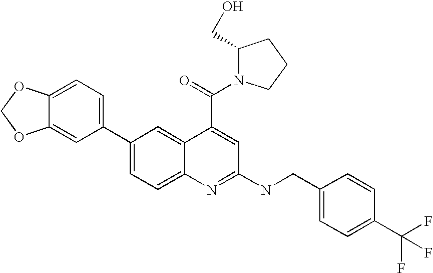 |
| 363 | 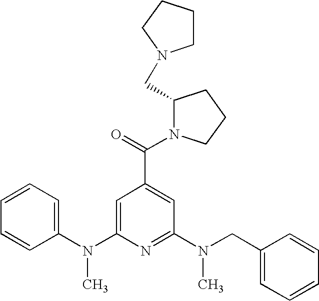 |
| 364 | 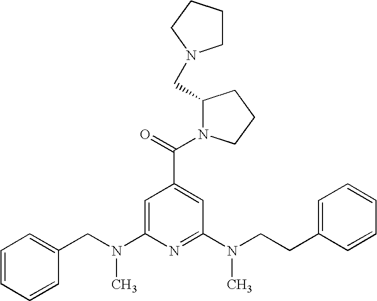 |
| 365 | 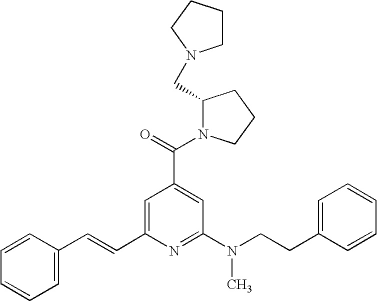 |
| 366 | 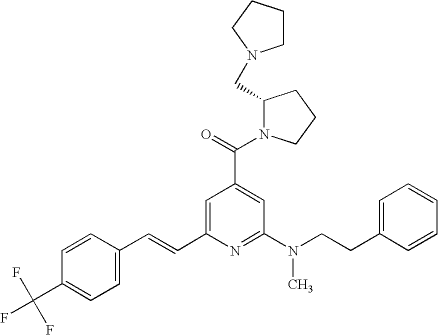 |
| 367 | 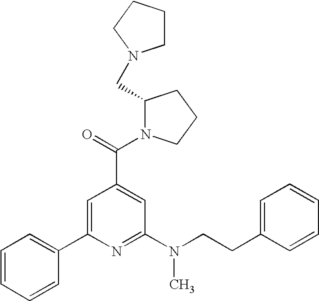 |
| 368 | 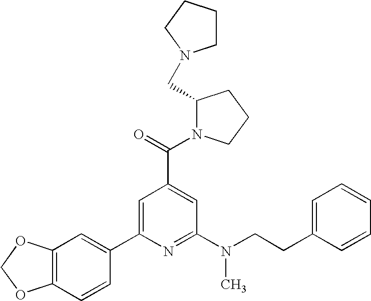 |
| 369 | 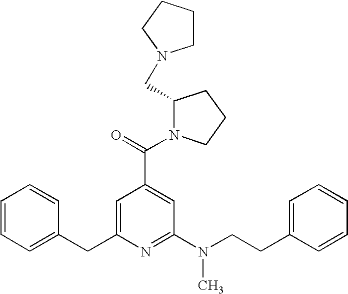 |
| 370 | 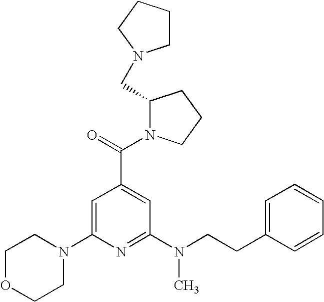 |
| 371 | 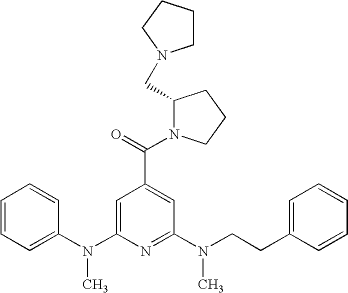 |
| 372 | 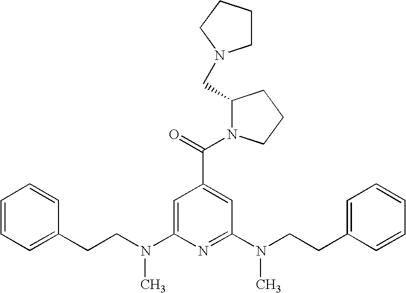 |
| 373 | 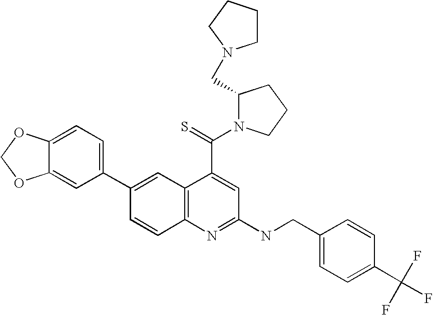 |
| 374 | 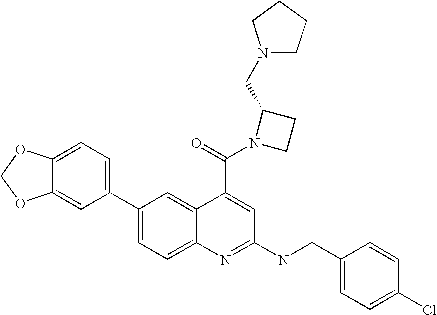 |
| 375 | 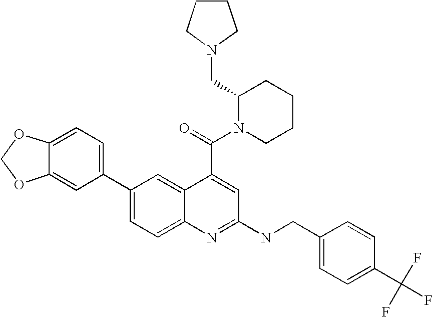 |
| 376 | 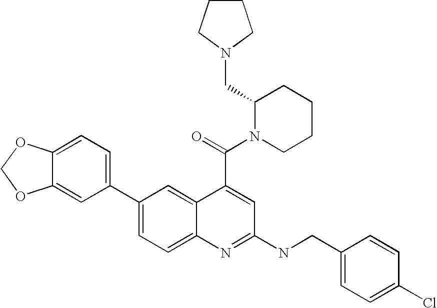 |
| 377 | 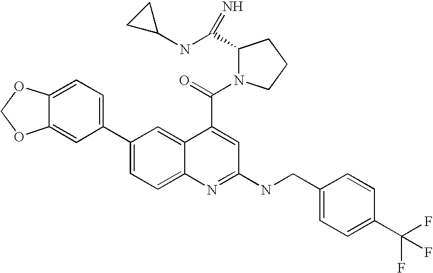 |
| 378 | 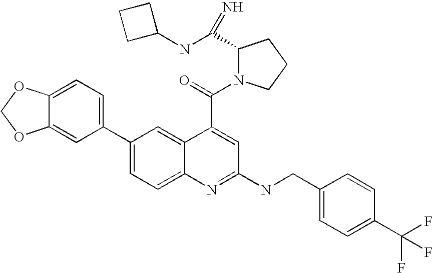 |
| 379 | 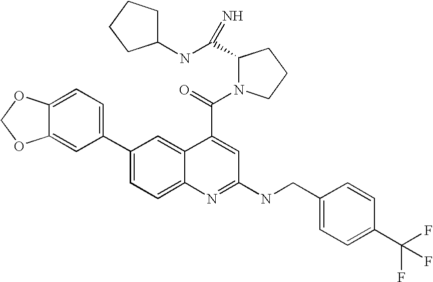 |
| 380 | 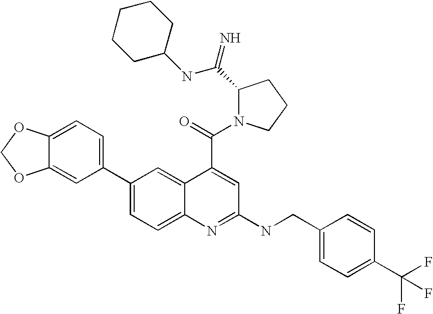 |
| 381 | 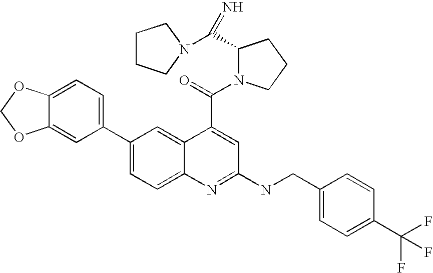 |
| 382 | 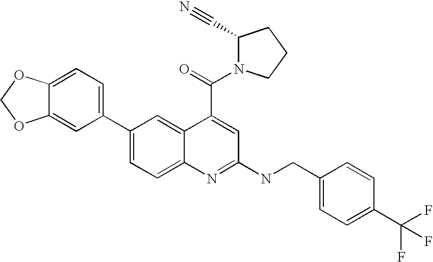 |
| 383 | 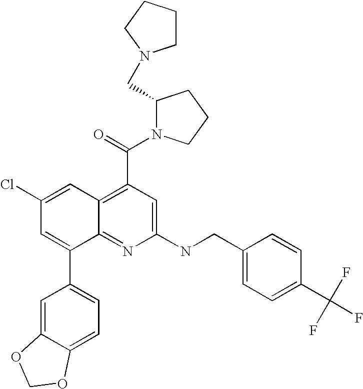 |
| 384 | 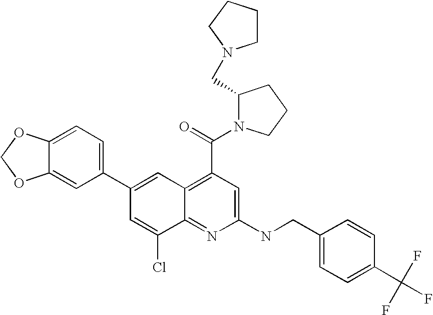 |
| 385 | 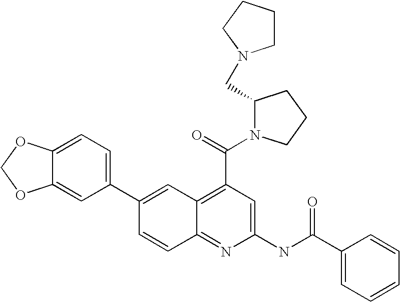 |
| 386 | 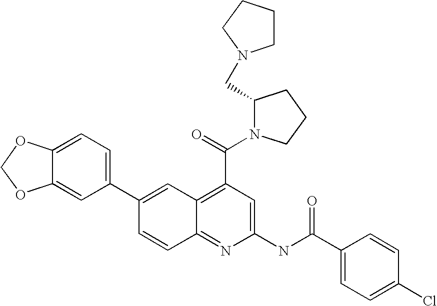 |
| 387 | 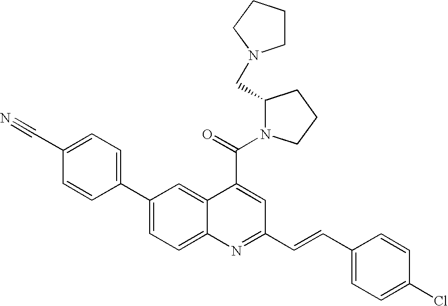 |
| 388 | 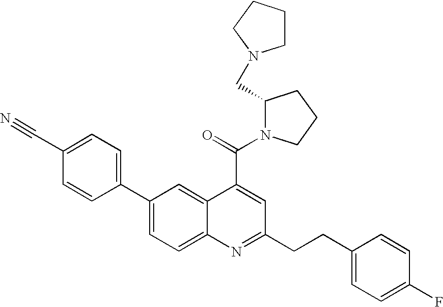 |
| 389 | 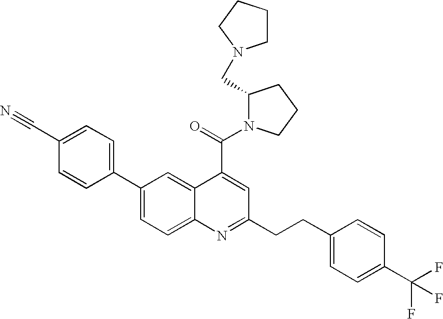 |
| 390 | 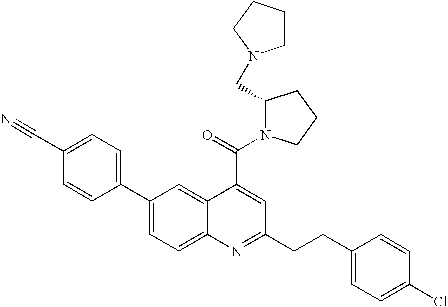 |
| 391 | 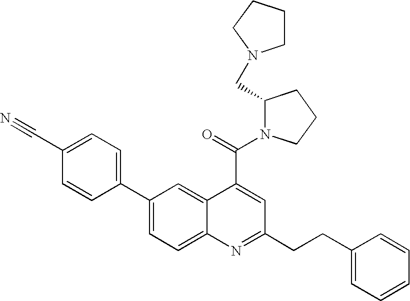 |
| 392 | 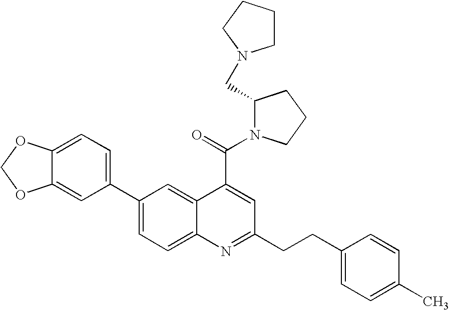 |
| 393 | 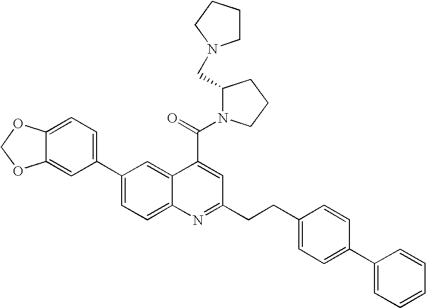 |
| 394 | 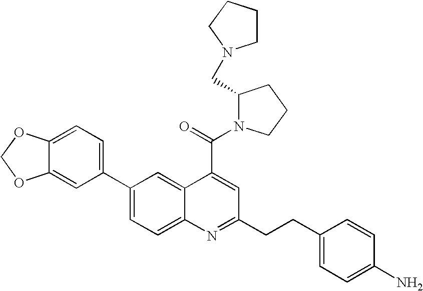 |
| 395 | 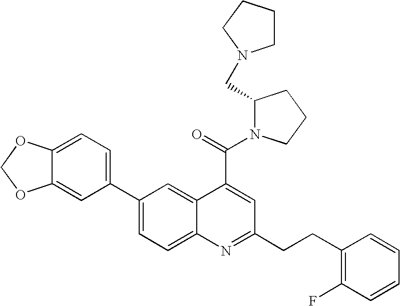 |
| 396 | 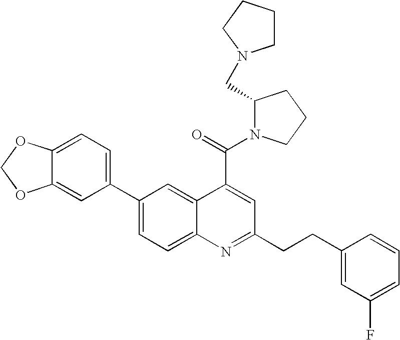 |
| 397 | 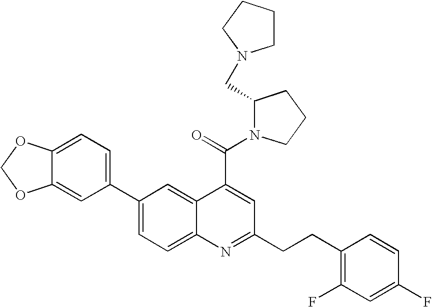 |
| 398 | 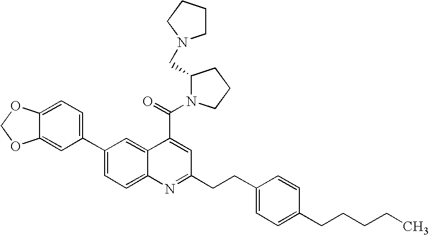 |
| 399 | 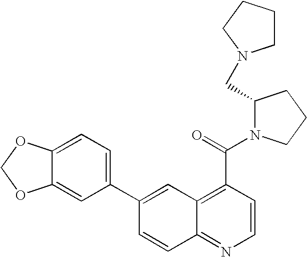 |
| 400 | 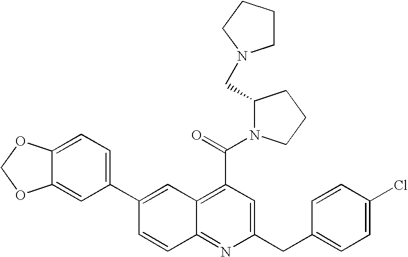 |
| 401 | 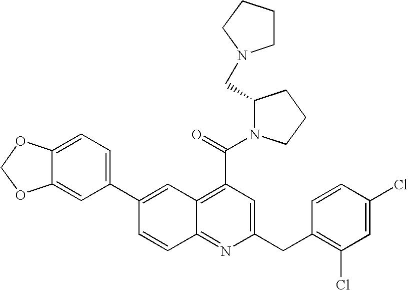 |
| 402 | 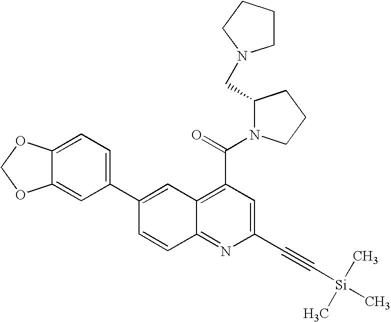 |
| 403 | 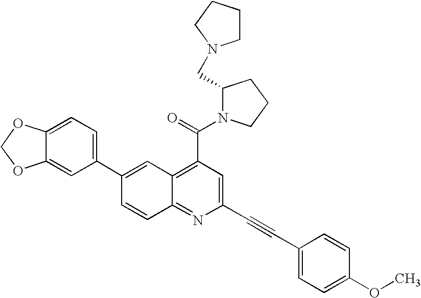 |
| 404 | 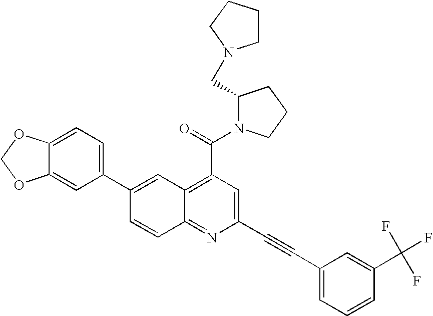 |
| 405 | 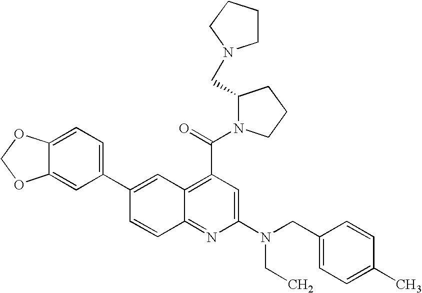 |
| 406 | 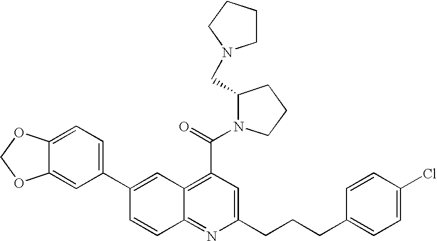 |
| 407 | 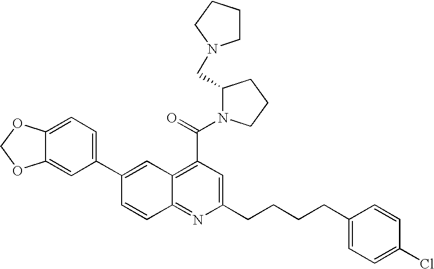 |
| 408 | 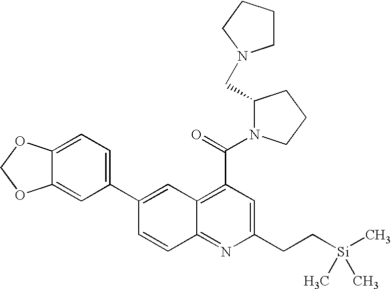 |
| 409 | 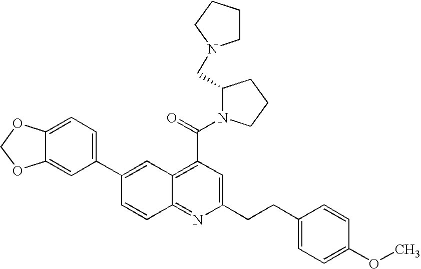 |
| 410 | 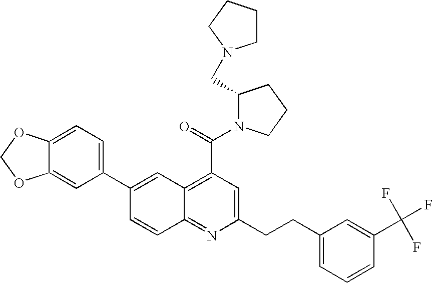 |
| 411 | 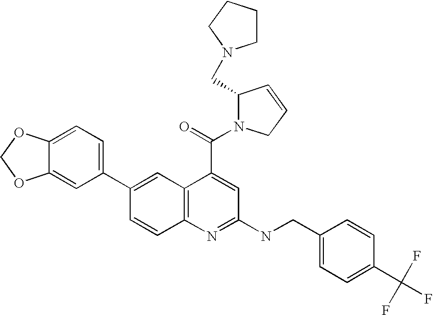 |
| 412 | 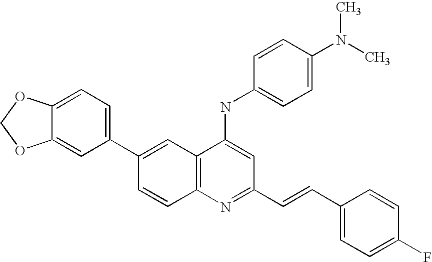 |
| 413 | 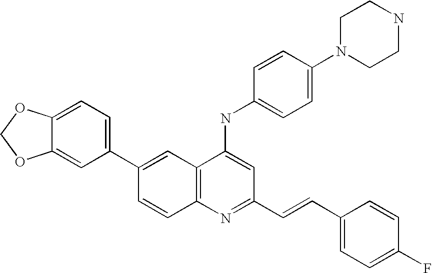 |
| 414 | 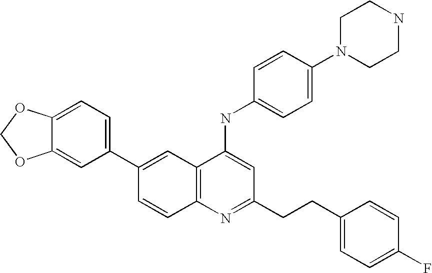 |
| 415 | 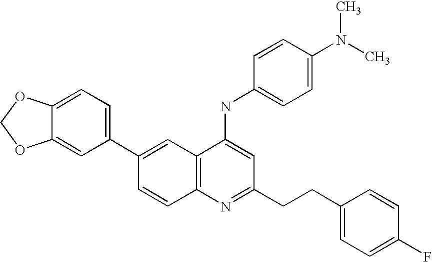 |
| 416 | 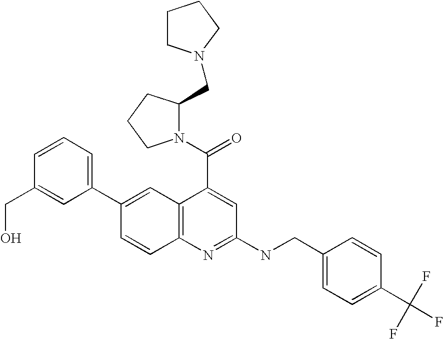 |
| 417 | 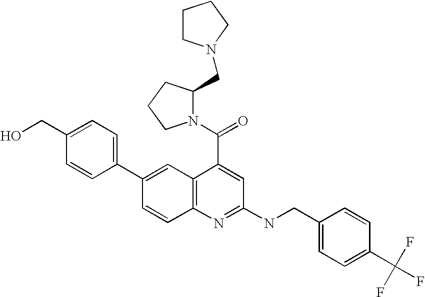 |
| 418 | 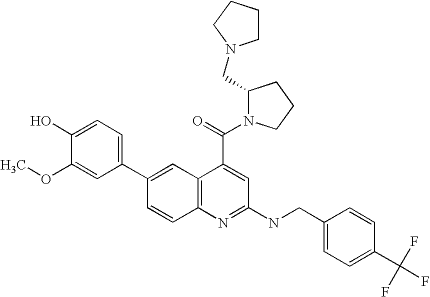 |
| 419 | 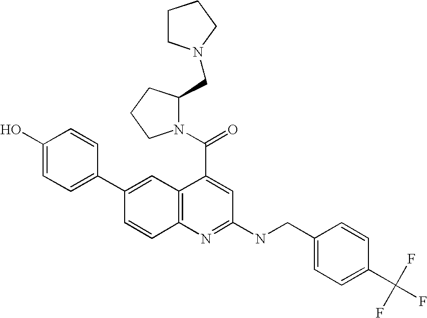 |
| 420 | 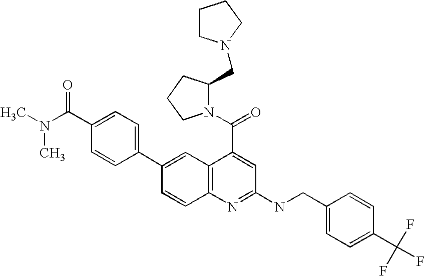 |
| 421 | 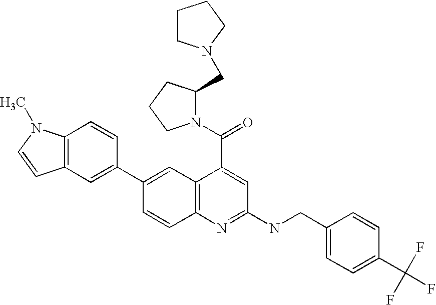 |
| 422 | 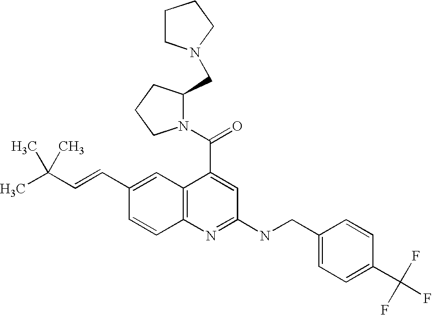 |
| 423 | 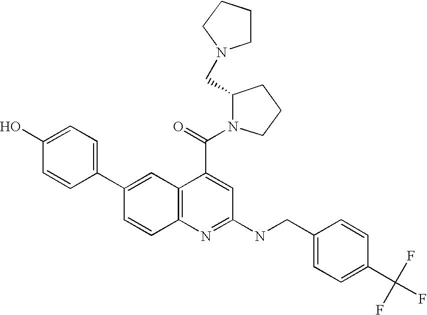 |
| 424 | 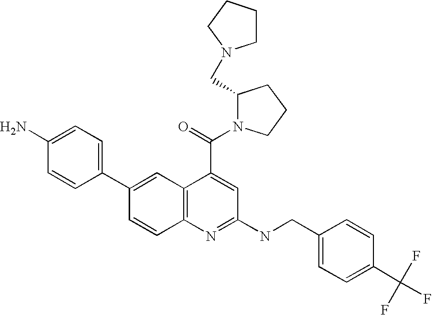 |
| 425 | 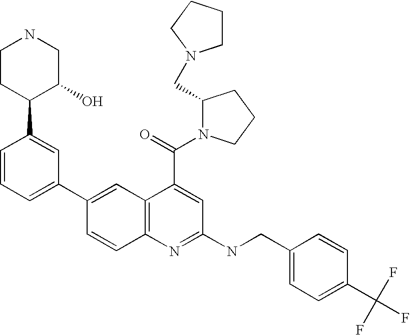 |
| 426 | 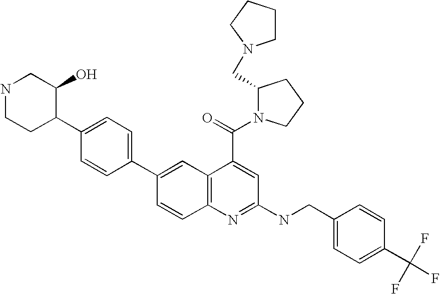 |
| 427 | 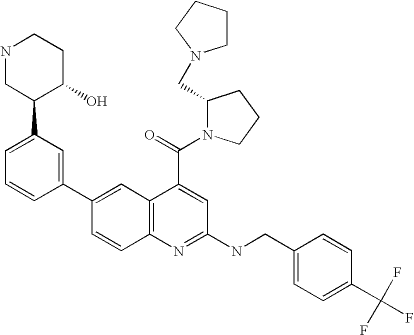 |
| 428 | 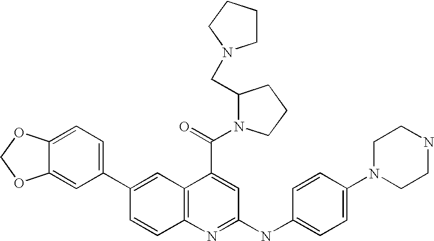 |
| 429 | 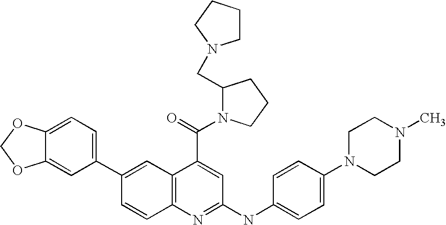 |
| 430 | 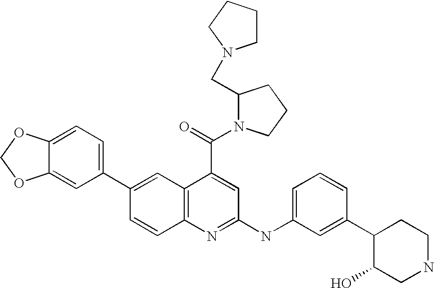 |
| 431 | 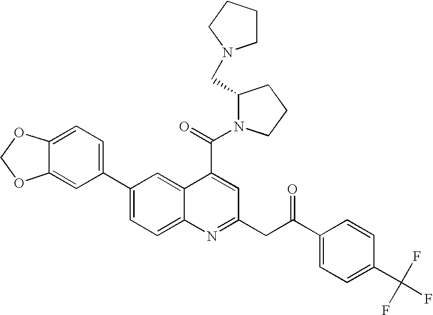 |
| 432 | 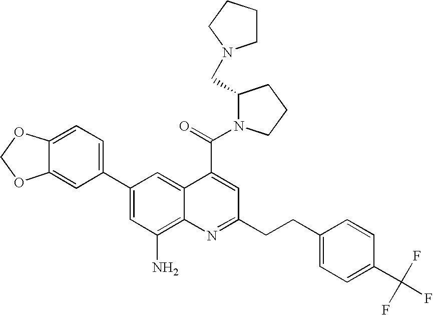 |
| 433 | 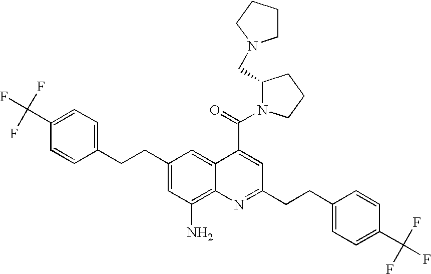 |
| 434 | 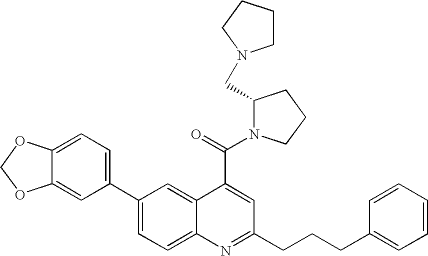 |
| 435 | 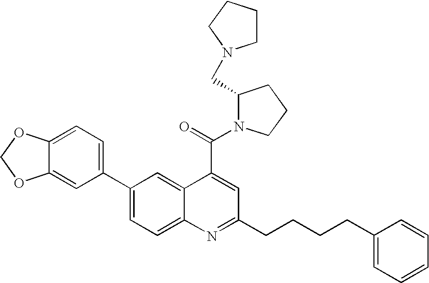 |
| 436 | 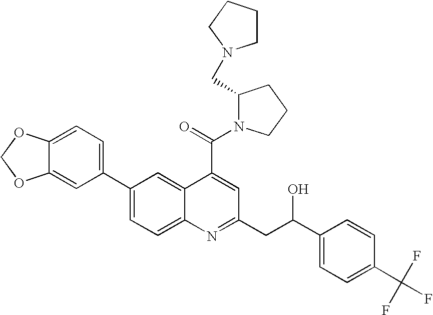 |
| 437 | 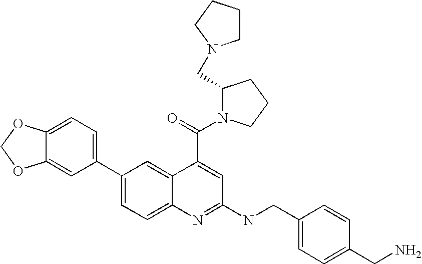 |
| 438 | 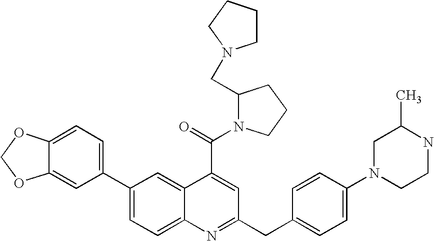 |
| 439 | 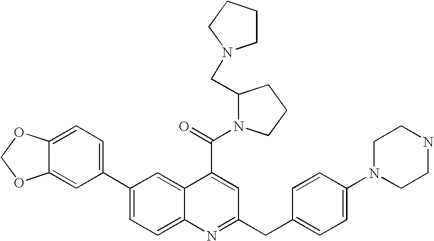 |
| 440 | 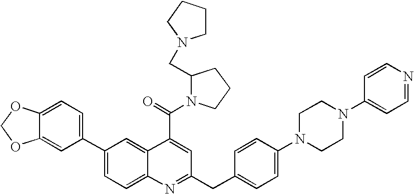 |
| 441 | 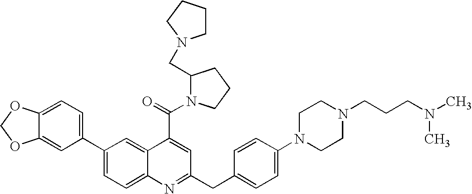 |
| 442 | 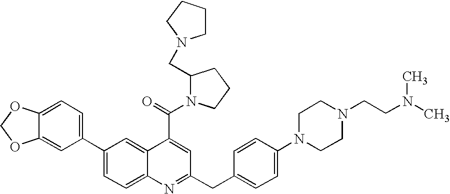 |
| 443 | 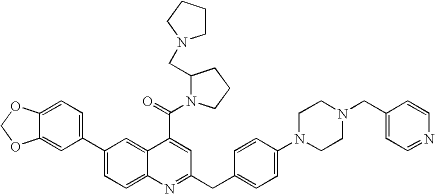 |
| 444 | 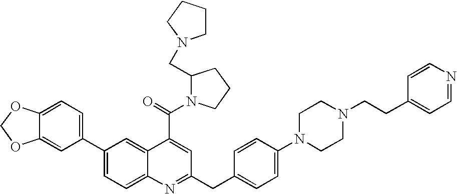 |
| 445 | 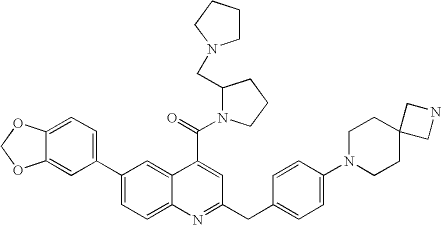 |
| 446 | 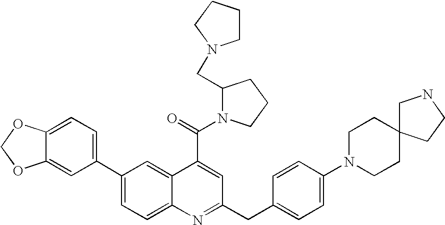 |
| 447 | 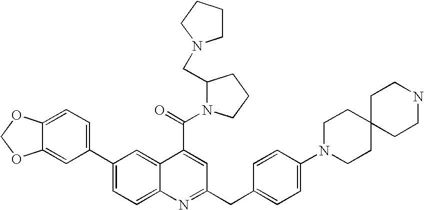 |
| 448 | 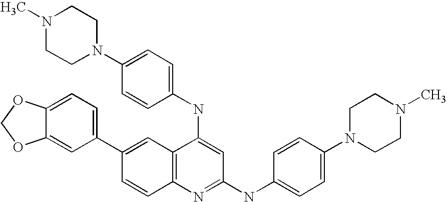 |
| 449 | 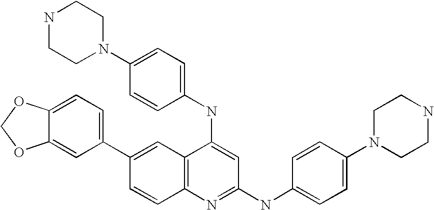 |
| 450 | 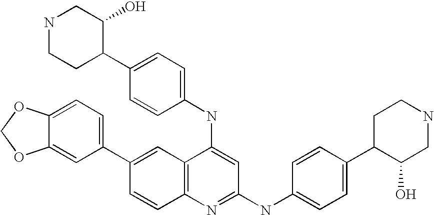 |

**In the structures above, the hydrogen atoms bonded to secondary nitrogen atoms are not depicted.

The compounds, compositions and methods described herein are useful in inhibiting ZAP-70 and Syk. Accordingly, the compounds, compositions and methods described are useful in treating ZAP-70 and Syk mediated diseases or disease symptoms in a mammal, in particular a human. ZAP-70 and Syk mediated diseases are exemplified by the following: lupus, organ transplant rejection, and inflammatory disorders. Examples of inflammatory disorders include arthritis (e.g., rheumatoid arthritis, rheumatoid spondylitis, gouty arthritis, traumatic arthritis, rubella arthritis, psoriatic arthritis, or osteoarthritis), inflammatory bowel disease, and Crohn's disease.

The compounds, compositions and methods of the present invention are useful in treating, for example, lupus, organ transplant rejection (e.g., kidney, liver, heart, lunch, pancreas (islet cells), bone marrow, cornea, small bowel, skin allografts or xenografts), and inflammatory disorders. Examples of inflammatory disorders include, but are not limited to, arthritis (e.g., rheumatoid arthritis, rheumatoid spondylitis, gouty arthritis, traumatic arthritis, rubella arthritis, psoriatic arthritis, or osteoarthritis), asthma, allergy, eczema, inflammatory bowel disease, and Crohn's disease.

Other aspects of this invention relate to a composition having a compound of any of the formulae described herein and a pharmaceutically acceptable carrier, or a compound of any of the formulae described herein, an additional therapeutic agent (e.g., anti-inflammatory; non-steroidal anti-inflammatory drugs (NSAID); steroid, and the like), and a pharmaceutically acceptable carrier; or a compound of any of the formulae described herein, an additional therapeutic agent, and a pharmaceutically acceptable carrier, wherein the additional therapeutic agent is an kinase binding agent (e.g., a polypeptide, an antibody or organic molecule).

Yet another aspect of this invention relates to a method of treating a subject (e.g., mammal) having a kinase mediated disorder or disorder symptom (including, but not limited to pain and inflammation). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The invention further relates to a product (i.e., a compound of any of the formulae herein) made by the methods described above.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

### Synthesis of Heterocyclic Kinase Inhibitors

Compounds of the formula (II) (where variables for all formulae (e.g., formula (II)) are as defined herein) are prepared by treating the compound of formula (IX) with malonic acid to provide a ring expansion compound, which is then coupled with a compound containing R² to provide the compound of formula (XII).

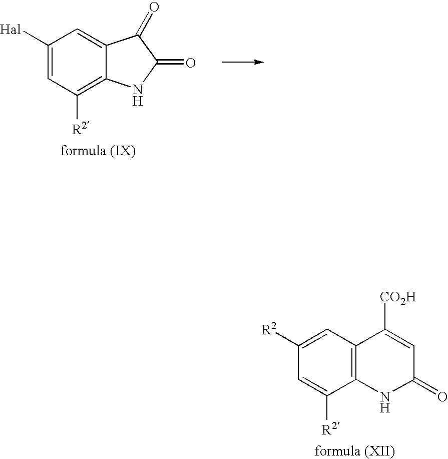

The compound of formula (XII) is treated with a chlorinating agent, such as POCl₃, and the carboxylic acid is coupled with a heterocyclic (e.g., heteroaryl or heterocyclyl) compound (“Het”) to provide a compound of formula (XIII).

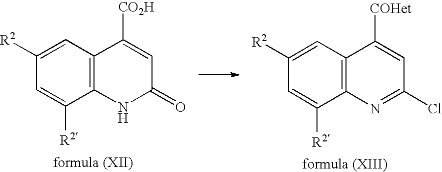

The resulting compound is then coupled with one or more coupling agents to provide a compound of formula (II).

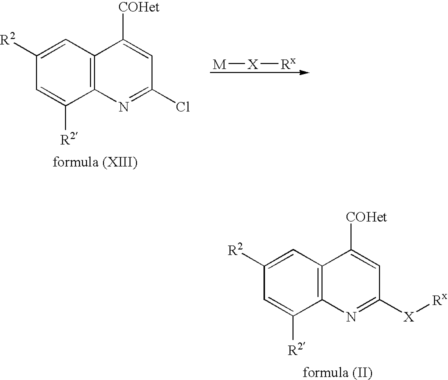

While the example above is shown with a quinoline core, other heterocyclic cores can also be used. For example, the synthesis above can be performed by substituting the exemplary heterocycles below for the starting material of formula (IX) above:

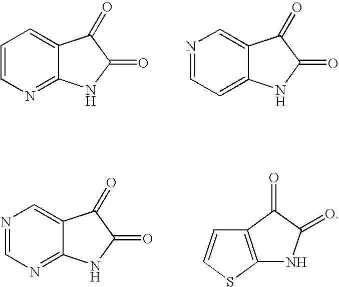

### These compounds are merely illustrative and are not intended to limit the scope of synthetic schemes.

The term “coupling agent” means a chemical agent that is used in a reaction that forms a bond between one chemical moiety and another moiety from the coupling agent. Some examples of coupling agents include transition metals such as Pd, Cu, and Mg and transition metal catalysts, as well as boron containing compounds such as boranes. Coupling agents can also include nucleophiles such as amines, alkoxides, sulfides or corresponding protonated forms.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

The compounds of the formulae described herein can, for example, be administered by injection, intravenously, intraarterially, subdermally, intraperitoneally, intramuscularly, or subcutaneously, or orally, buccally, nasally, transmucosally, topically, in an ophthalmic preparation, or by inhalation, with a dosage ranging from about 0.001 to about 100 mg/kg of body weight, preferably dosages between 10 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

Pharmaceutical compositions of this invention comprise a compound of the formulae described herein or a pharmaceutically acceptable salt thereof, an additional agent including for example, a steroid or an analgesic; and any pharmaceutically acceptable carrier, adjuvant or vehicle. Alternate compositions of this invention comprise a compound of the formulae described herein or a pharmaceutically acceptable salt thereof; and a pharmaceutically acceptable carrier, adjuvant or vehicle. The compositions delineated herein include the compounds of the formulae delineated herein, as well as additional therapeutic agents if present, in amounts effective for achieving a modulation of disease or disease symptoms, including kinase mediated disorders or symptoms thereof. The compositions are made by methods including the steps of combining one or more compounds delineated herein with one or more carriers and, optionally, one or more additional therapeutic agents delineated herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

In some cases, the pH of the formulation may be adjusted with pharmaceutically acceptable acids, bases or buffers to enhance the stability of the formulated compound or its delivery form.

The term parenteral as used herein includes subcutaneous, intracutaneous, intravenous, intramuscular, intraarticular, intraarterial, intrasynovial, intrasternal, intrathecal, intralesional and intracranial injection or infusion techniques.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

### EXAMPLES

### Example 1

### Synthesis of 2-Aminoquinolines

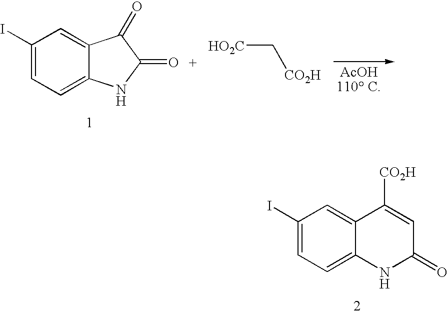

5-Iodoisatin 1 (10 g, 36.3 mmol) and malonic acid (7.5 g, 72 mmol) in 200 mL of glacial acetic acid were refluxed overnight. The precipitate was collected by filtration and washed with AcOH and acetone. The solid was then refluxed with EtOH for 1 h. Filtration and washing with EtOH and Et₂O gave 6-Iodo-2-oxo-1,2-dihydro-quinoline-4-carboxylic acid 2 as the product, yielding 8.8 g (76%). ¹H-NMR (400 MHz, DMSO-d₆): δ 14.0 (br s, 1H), 12.13 (s, 1H), 8.56 (d, 1H, J=8.1 Hz); 7.83 (dd, 1H, J=8.7, 1.8 Hz), 7.17 (d, 1H, J=8.4 Hz), 6.93 (s, 1H).

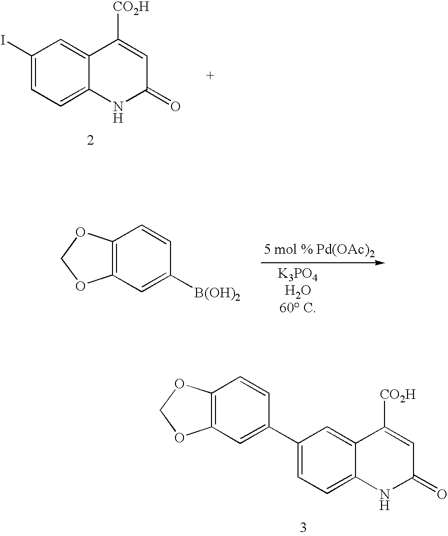

A mixture of 6-Iodo-2-oxo-1,2-dihydro-quinoline-4-carboxylic acid 2 (3.15 g, 10 mmol), 3,4-(methylenedioxyl)phenylboronic acid (2.49 g, 15 mmol), K₃PO₄ (8.49 g, 40 mmol), and Pd(OAc)₂ (112 mg, 0.5 mmol) in 60 mL of degassed H₂O was heated under argone at 60° C. for 2 h. After cooling to room temperature, the solid was collected by filtration, washed with H₂O and acetone. It was then treated with 20 mL of 1M HCl, the resulting greenish yellow solid was filtered again and washed with H₂O. Drying in vacuo over P₂O₅ yielded 2.53 g (82%) of product 3 as a greenish yellow solid. ¹H-NMR (400 MHz, DMSO-d₆): δ 14.0 (br s, 1H), 12.11 (s, 1H), 8.34 (s, 1H), 7.79 (d, 1H, J=7.6 Hz), 7.40 (d, 1H, J=8.6 Hz), 7.18 (s, 1H), 7.08 (d, 1H, J=7.1 Hz), 7.01 (d, 1H, J=8.2 Hz), 6.91 (s, 1H), 6.06 (s, 2H).

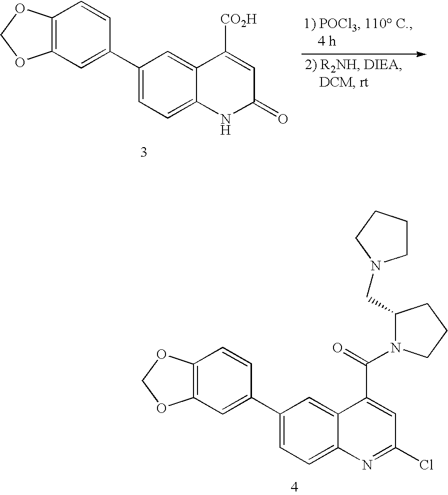

6-Benzo[1,3]dioxol-5-yl-2-oxo-1,2-dihydro-quinoline-4-carboxylic acid 3 (1.0 g, 3.2 mol) in 10 mL of oxyphosphourus chloride was refluxed for 4 h and cooled to room temperature. The solution was concentrated to dryness to yield a brownish yellow solid. The solid was then dissolved in 20 mL of methylene dichloride. Diisopropylethylamine (1.50 g, 11.5 mmol) and 2-(S)-pyrroldinylmethyl pyrrolidine (0.59 g, 3.84 mmol) were slowly added into the solution at 0° C. The mixture was allowed to stir at room temperature for 12 h. After removing the solvent by rotary evaporation, the residue was dissolved in ethyl acetate, washed with saturated aqueous NaHCO₃ and brine. The organic phase was dried over sodium sulfate and concentrated. The residue was purified by silica gel column chromatography (Et₃N—AcOBt 5:95) to give (6-Benzo[1,3]dioxol-5-yl-2-chloro-quinolin-4-yl)-(2-pyrrolidin-1-ylmethyl-pyrrolidin-1-yl)-methanone 4 (1.20 g, 81%). MS m/z 464.2 (M⁺+1); ¹H-NMR (400 MHz, CDCl₃): δ 8.05 (m, 1H), 7.93 (m, 1H), 7.85 (br s, 1H), 7.41 (s, 1H), 7.13 (m, 1H), 7.11 (s, 1H), 6.92 (m, 1H), 6.03 (s, 1H), 4.58 (m, 1H), 3.92 (m, 0.5H), 3.75 (m, 0.5H), 3.38-3.13 (m, 2H), 2.94 (m, 3H), 2.35-2.15 (m, 2H), 2.04 (m, 3H), 2.01-1.85 (m, 4H), 1.83 (m, 1H).

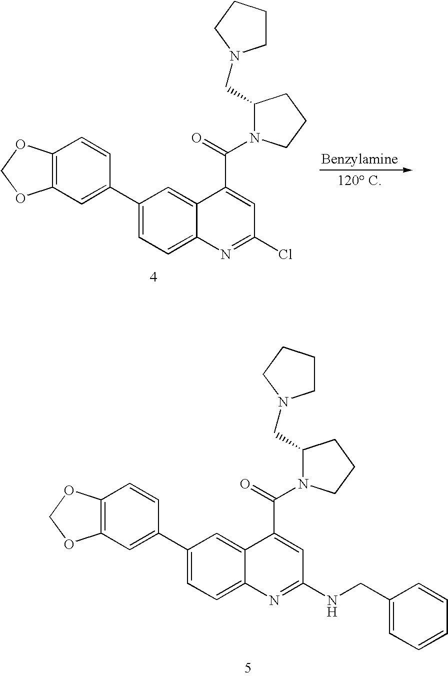

2-Chloroquinoline 4 (80 mg, 0.17 mg) in benzylamine (0.3 mL) was heated for 12 h at 120° C. LC-MS analysis indicated that the reaction was complete. The reaction mixture was then dissolved in 3 mL of DMSO/CH3CN (3:1), and purified by preparative LC to yield product 5. MS m/z 535.3 (M⁺+1).

### Example 2

### Synthesis of 2-Alkyl Quinolines

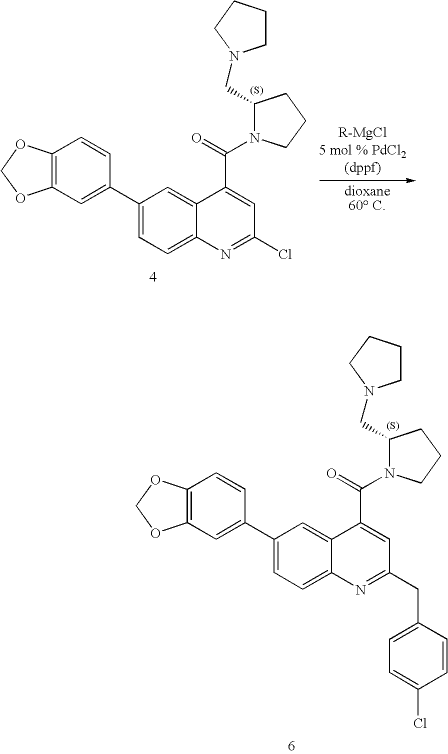

The solution of 4-chlorobenzyl magnesium chloride (0.43 mL, 0.25 M solution in Et₂O) was added into a mixture of 2-chloroquinoline 4 (25 mg, 0.054 mg) and PdCl₂(dppf) (2.2 mg, 0.0027 mmol) in 0.5 mL of dioxane under argone. The reaction mixture was stirred at 100° C. for 12 h. After cooling to room temperature, aqueous NH₄Cl was added. The mixture was extracted with EtOAc, and washed with brine. The organic layer was dried over Na₂SO₄, and concentrated. The residue was purified by preparative LC to give product 6. MS m/z 554.3 (M⁺+1).

### Example 3

### Synthesis of 6-Substituted Quinolines

### Procedure A:

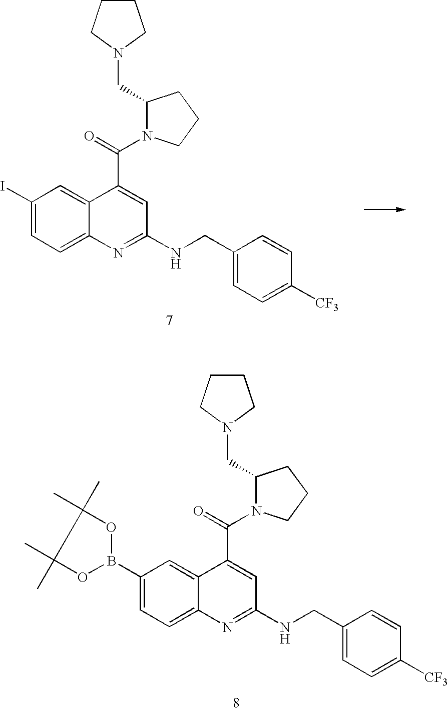

To a 25 ml round bottom flask charged with bis(pinacolato)diboron (279 mg, 1.1 mmol), KOAc (294, 3.0 mmol) and PdCl₂(dppf) (24.5 mg, 0.03 mmol) was added a solution of 6-iodoquinoline 7 (607 mg, 1.0 mmol) in DMSO (6 mL). The mixture was thoroughly degassed by alternately connected the flask to vacuum and Argon. This resulting mixture was then heated at 80° C. overnight, diluted by EtOAc (40 mL) and filtered through CELITE. The resulting product 8 was used in next steps without further purification after concentration. Molecular weight is 608.3 and LC-MS showed 609.2 (M⁺+1).

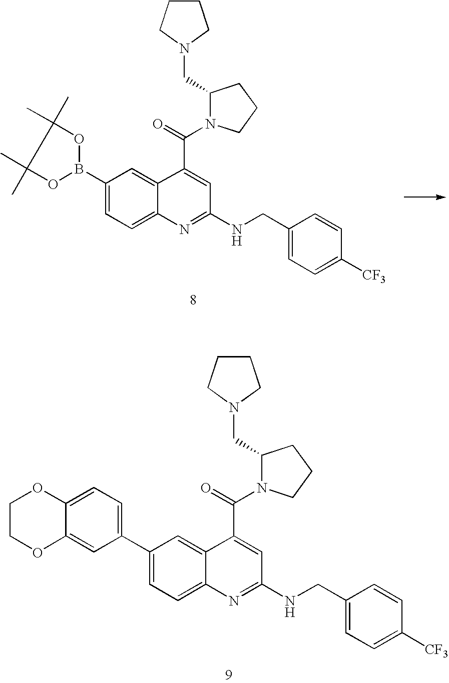

Under Argon, the 6-boronate 8 (15 mg, 0.025 mmol) in dioxane (2.0 mL) was added to the flask which was charged with Pd(dppf)Cl₂ (2 mg), Cs₂CO₃ (17 mg, 0.055 mmol), and 3,4-ethylenedioxyiodobenzene (15 mg, 0.057 mmol). The mixture was thoroughly degassed by alternately connected the flask to vacuum and Argon. The resulting solution was heated to 70° C. and stirred overnight. It was diluted by EtOAc after cooled to room temperature. The solid was removed by filter through CELITE and washed by some EtOAc. Concentration to remove the solvent and the resulting residue purified by LC to give product 9. Exact Mass is 616 and LC-MS showed 617 (M⁺+1).

### Procedure B:

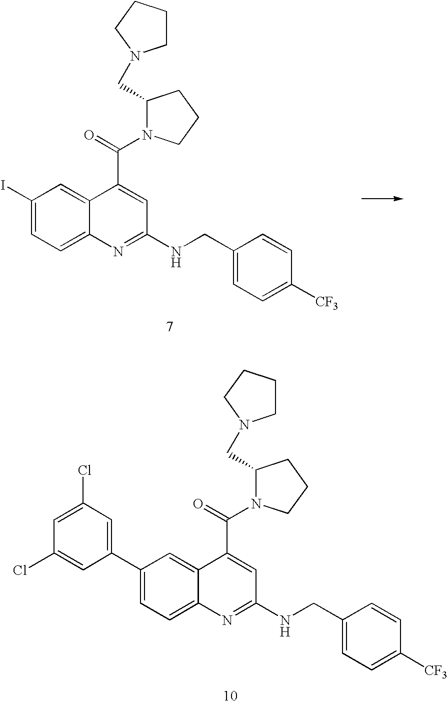

A reaction vessel was charged with 6-iodoquinoline 7 (25.0 mg, 0.0411 mmol, 1.00 equiv), Pd(dppf)Cl₂ (1.5 mg, 0.0021, 0.050 equiv), K₃PO₄ (35.0 mg, 0.164 mmol, 4.00 equiv) and 3,5-dichlorophenylboronic acid (15.7 mg, 0.0811 mmol, 2.00 equiv). After flushing the vessel with argon, dioxane (2.0 mL) was introduced under argon and the resulting suspension was stirred overnight at 80° C. The crude reaction mixture was allowed to cool to rt, filtered through CELITE with the aid of EtOAc and concentrated. The crude residue was purified by LC to give product 10. LC-MS m/z 627.2 (M⁺+1).

An identical procedure was used for the synthesis of 6-aryl quinolines from the corresponding 6-bromo quinoline.

### Procedure C:

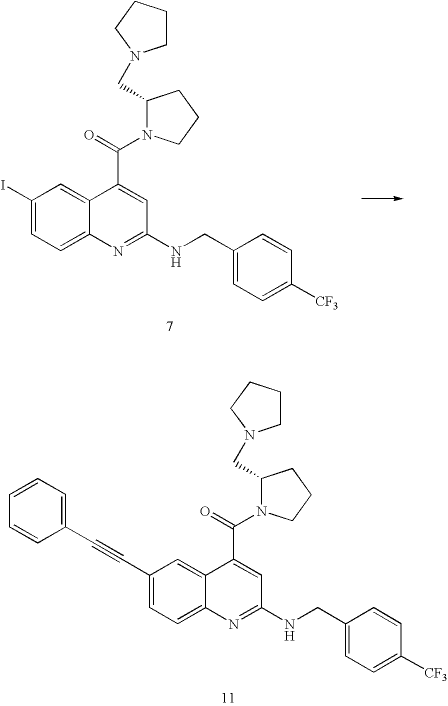

To a round bottom flask charged with the 6-iodoquinoline 7 (61 mg, 0.1 mmol), phenylacetylene (1 molar equiv.), Pd(PPh₃)₂Cl₂ (4.0 mg), and CuI (1.0 mg) was added Et₃N (2 mL). The mixture was thoroughly degassed by alternately connected the flask to vacuum and Argon and then heated to 45-50° C. and stirred overnight. The mixture was diluted with EtOAc (20 ml) after cooling to room temperature and filtered through CELITE. After concentration, the crude product 11 was used in the next step without further purification. A small portion was purified by LC. Exact Mass is 582 and LC-MS showed 583 (M⁺+1).

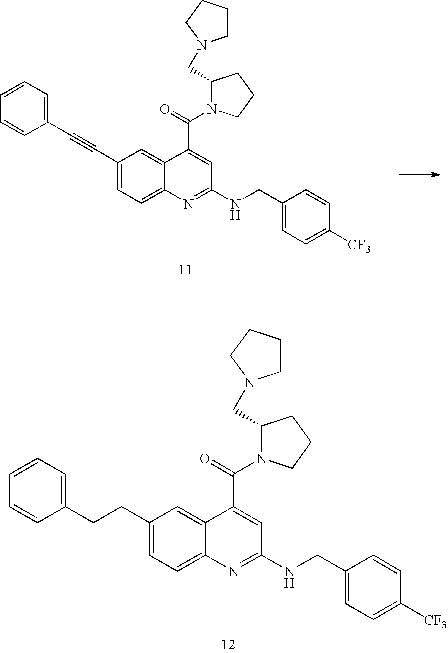

Under H₂ (1 atm), the crude acetylene compound II (15 mg) was reduced with H₂ in the presence of 5% Pd/C (5 mg) in methanol at room temperature overnight. The mixture was filtered through CELITE to remove the catalyst and concentrated under reduced pressure to give the crude product. Compound 12 was further purified by LC. The Exact Mass is 586 and LC-MS showed 587 (M⁺+1).

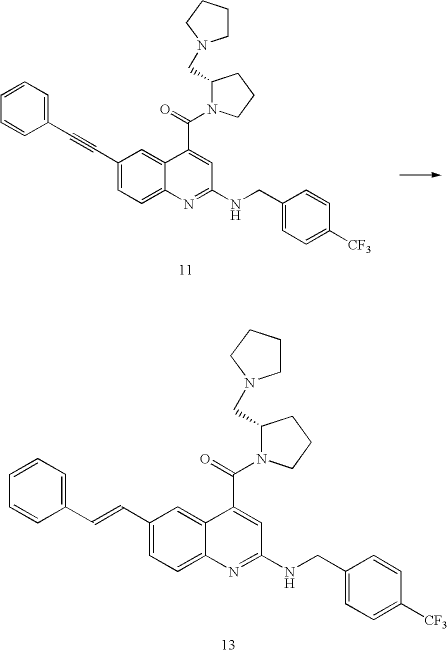

Under H₂ (1 atm), the crude acetylene compound II (15 mg) was reduced in the presence of 5% palladium on barium sulfate (5 mg) in methanol at room temperature overnight. The mixture was filtered through CELITE to remove the catalyst and concentrated under reduced pressure to give the crude product. Preparative LC gave the pure product 13 for testing. The Exact Mass is 584 and LC-MS showed 585 (M⁺+1).

### Example 4

### Synthesis of 2-Thioquinolines

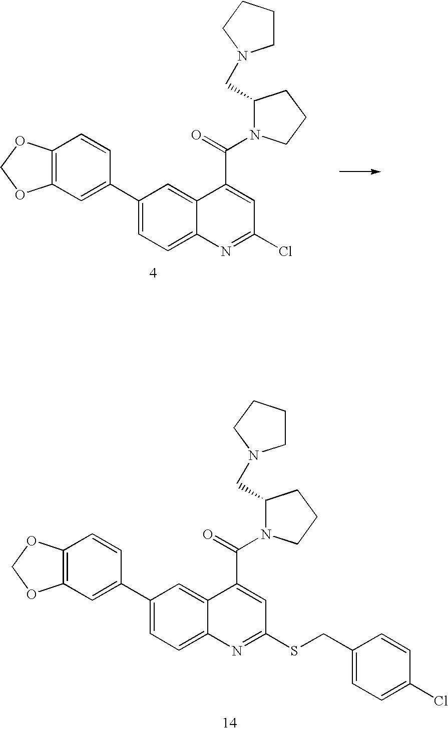

Under Argon, to the solution of 2-chloroquinoline 4 (15 mg, 0.032 mmol) and 4-chlorobenzyl mercaptan (8.6 mg, 0.048 mmol) in dry DMF (1.0 mL) was added Cs₂CO₃ (16 mg, 0.048 mmol). The resulting mixture was heated up to 80° C. and stirred at this temperature for two hours. After cooled to room temperature, EtOAc (20 ml) was added to dilute the mixture and the organic phase was washed by water and brine and dried over Na₂SO₄. After concentration, the residue was purified by LC to yield product 14. The Exact Mass is 586 and LC-MS showed 587 (M⁺+1).

### Example 5

### DELFIA Assay

Before initiation of kinase reactions, compounds were pre-incubated with ZAP-70. Pre-incubation reactions contained 62.5 mM Mops pH 7.0, 12.5 mM MgCl₂, 12.5% glycerol, 3.1 nM ZAP-70, 62.5 nM biotinylated poly(glu,Tyr), 0.1 mg/ml BSA, 6.25% DMSO and 0-100 mM compound in a total volume of 40 ml. After a 10 minute room temperature incubation, 10 ml of 5 mM ATP was added to start the reaction. Reactions were incubated at room temperature for 30 minutes then terminated by addition of 5 ml 500 mM EDTA. The amount of phosphate transferred to biotinylated poly(glu,tyr) was measured using the Dissociation Enhanced Lanthanide Fluorescence Immuno-assay (DELFIA) from Perkin Elmer according to manufacturers protocol. Briefly, biotinylated poly (gly,tyr) was captured on streptavidin coated plates, washed twice, then incubated with Europium labeled-anti-phosphotyrosine antibody. Free antibody was removed with six washes, Europium was dissociated from the antibody, and Europium fluorescence was measured at using an excitation wavelength of 340 nM and an emission wavelength of 615 nM.

TABLE 2
In vitro Activity of Representative Compounds*

| Number | Activity |
|---|---|
| 1 | B |
| 2 | B |
| 3 | A |
| 4 | C |
| 5 | B |
| 6 | A |
| 7 | A |
| 8 | A |
| 9 | A |
| 10 | A |
| 11 | B |
| 12 | C |
| 13 | C |
| 14 | A |
| 15 | C |
| 16 | C |
| 17 | C |
| 18 | C |
| 19 | C |
| 20 | A |
| 21 | A |
| 22 | C |
| 23 | C |
| 24 | C |
| 25 | C |
| 26 | A |
| 27 | C |
| 28 | A |
| 29 | B |
| 30 | C |
| 31 | C |
| 32 | A |
| 33 | B |
| 34 | B |
| 35 | C |
| 36 | C |
| 37 | C |
| 38 | B |
| 39 | B |
| 40 | C |
| 41 | C |
| 42 | B |
| 43 | C |
| 44 | C |
| 45 | C |
| 46 | C |
| 47 | B |
| 48 | B |
| 49 | B |
| 50 | C |
| 51 | C |
| 52 | C |
| 53 | C |
| 54 | C |
| 55 | C |
| 56 | C |
| 57 | C |
| 58 | B |
| 59 | B |
| 60 | C |
| 61 | B |
| 62 | B |
| 63 | B |
| 64 | B |
| 65 | C |
| 66 | B |
| 67 | A |
| 68 | C |
| 69 | A |
| 70 | C |
| 71 | A |
| 72 | B |
| 73 | B |
| 74 | B |
| 75 | B |
| 76 | C |
| 77 | C |
| 78 | B |
| 79 | B |
| 80 | A |
| 81 | B |
| 82 | B |
| 83 | B |
| 84 | A |
| 85 | B |
| 86 | B |
| 87 | A |
| 88 | A |
| 89 | B |
| 90 | B |
| 91 | C |
| 92 | C |
| 93 | A |
| 94 | B |
| 95 | B |
| 96 | B |
| 97 | B |
| 98 | C |
| 99 | C |
| 100 | A |
| 101 | B |
| 102 | B |
| 103 | B |
| 104 | B |
| 105 | C |
| 106 | C |
| 107 | C |
| 108 | B |
| 109 | B |
| 110 | A |
| 111 | A |
| 112 | A |
| 113 | A |
| 114 | A |
| 115 | A |
| 116 | B |
| 117 | C |
| 118 | B |
| 119 | A |
| 120 | B |
| 121 | B |
| 122 | B |
| 123 | B |
| 124 | B |
| 125 | C |
| 126 | C |
| 127 | C |
| 128 | A |
| 129 | B |
| 130 | B |
| 131 | B |
| 132 | B |
| 133 | B |
| 134 | B |
| 135 | B |
| 136 | B |
| 137 | B |
| 138 | A |
| 139 | B |
| 140 | C |
| 141 | B |
| 142 | B |
| 143 | B |
| 144 | B |
| 145 | B |
| 146 | B |
| 147 | B |
| 148 | B |
| 149 | B |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | A |
| 277 | A |
| 278 | A |
| 279 | A |
| 280 | C |
| 281 | C |
| 282 | B |
| 283 | C |
| 284 | C |
| 285 | B |
| 286 | C |
| 287 | B |
| 288 | B |
| 289 | B |
| 290 | B |
| 291 | A |
| 292 | C |
| 293 | B |
| 294 | C |
| 295 | B |
| 296 | B |
| 297 | A |
| 298 | B |
| 299 | C |
| 300 | C |
| 301 | 1.785 μM |
| 302 | C |
| 303 | C |
| 304 | B |
| 305 | C |
| 306 | C |
| 307 | B |
| 308 | B |
| 309 | B |
| 310 | C |
| 311 | B |
| 312 | B |
| 313 | B |
| 314 | B |
| 315 | C |
| 316 | B |
| 317 | C |
| 318 | B |
| 319 | C |
| 320 | B |
| 321 | B |
| 322 | — |
| 323 | A |
| 324 | C |
| 325 | C |
| 326 | C |
| 327 | A |
| 328 | B |
| 329 | A |
| 330 | C |
| 331 | C |
| 332 | A |
| 333 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | C |
| 345 | A |
| 346 | A |
| 347 | A |
| 348 | C |
| 349 | C |
| 350 | A |
| 351 | C |
| 352 | C |
| 353 | A |
| 354 | A |
| 355 | A |
| 356 | A |
| 357 | B |
| 358 | C |
| 359 | C |
| 360 | B |
| 361 | B |
| 362 | A |
| 363 | B |
| 364 | B |
| 365 | B |
| 366 | B |
| 367 | A |
| 368 | A |
| 369 | A |
| 370 | A |
| 371 | A |
| 372 | B |
| 373 | B |
| 374 | B |
| 375 | B |
| 376 | B |
| 377 | A |
| 378 | A |
| 379 | A |
| 380 | A |
| 381 | A |
| 382 | A |
| 383 | A |
| 384 | C |
| 385 | B |
| 386 | B |
| 387 | B |
| 388 | B |
| 389 | C |
| 390 | A |
| 391 | A |
| 392 | C |
| 393 | C |
| 394 | A |
| 395 | C |
| 396 | C |
| 397 | C |
| 398 | C |
| 399 | A |
| 400 | C |
| 401 | C |
| 402 | C |
| 403 | C |
| 404 | C |
| 405 | C |
| 406 | C |
| 407 | C |
| 408 | A |
| 409 | A |
| 410 | C |
| 411 | C |
| 412 | A |
| 413 | B |
| 414 | A |
| 415 | A |
| 416 | A |
| 417 | A |
| 418 | A |
| 419 | A |
| 420 | A |
| 421 | A |
| 422 | A |
| 423 | A |
| 424 | A |
| 425 | A |
| 426 | A |
| 427 | A |
| 428 | C |
| 429 | C |
| 430 | C |
| 431 | B |
| 432 | C |
| 433 | C |
| 434 | C |
| 435 | C |
| 436 | C |
| 437 | C |
| 438 | C |
| 439 | C |
| 440 | C |
| 441 | C |
| 442 | C |
| 443 | C |
| 444 | C |
| 445 | C |
| 446 | C |
| 447 | C |
| 448 | C |
| 449 | C |

*“A” means the compound has an IC₅₀ of >10 μM in vitro, “B” means the compound has an IC₅₀ between 1 and 10 μM in vitro, and “C” means the compound has an IC₅₀ of <1 μM in vitro.

### Example 6

### Cell-Based Assay of ZAP-70-Mediated LAT Phosphorylation

ZAP-70 tyrosine kinase activity present in activated Jurkat T cells was measured by assessing the phosphorylation status of LAT. LAT is a palmitoylated p36/38 kDa plasma membrane-associated protein expressed in all T lymphocytes and it is the physiologic substrate of activated ZAP-70. When Jurkat cells are stimulated with an anti-CD3 monoclonal antibody, which crosslinks the surface antigen receptor-CD3 complex, ZAP-70 becomes activated and phosphorylates two tyrosine residues on LAT, Tyr-191 & Tyr-226. Therefore, assessment of LAT Tyr-191 and Tyr-226 phosphorylation in anti-CD3 stimulated Jurkat T cells is a specific measure of intracellular ZAP-70 activity. Accordingly, compounds that inhibit Tyr-191 and Tyr-226 phosphorylation of LAT indicate successful antagonism of ZAP-70 tyrosine kinase activity or upstream activation of the ZAP-70 pathway.

Jurkat cells (ATCC) cultured at 15-20×106 cells/mL are pre-incubated for 15 mins at 370° C. with inhibitor compounds, or carrier (DMSO), and then are stimulated for 5 min. by the addition of 1 mg/mL final concentration of anti-CD3 monoclonal antibody (UCHT-1 or OKT-3; E-Bioscience). The cells are then lysed by a detergent-based buffer and extracts are used for immunoprecipitation of all phosphotyrosine-containing proteins using a commercially available phosphotyrosine specific monoclonal antibody (4G10; Upstate Biotechnology Inc.) and protein-A-conjugated agarose beads. After washing the immune complexes to remove non-specifically bound proteins, the immunoprecipitated phosphotyrosine proteins are liberated from the agarose beads by boiling and denaturing and are resolved by SDS-PAGE and transferred to nitrocellulose membranes. LAT phosphorylated on Tyr-191 and Tyr-226 is then detected by standard western blotting technology using commercially available phospho-specific antibodies that react specifically with either phospho-Tyr-191 or phospho-Tyr-226 on LAT (Upstate Biotechnology Inc.), an enzyme-conjugated secondary antibody (horseradish peroxidase-conjugated goat anti-rabbit IgG antiserum), and a Storm Imaging system (Amersham-Pharmacia).

Inhibition was observed with representative compounds described herein.

### Example 7

### IL-2 Inhibition Cell-Based Assay

IL-2 production was measured in Jurkat cells following co-stimulation with Anti-CD3 and Anti-CD28 antibodies. Cells were seeded at 1×10⁵/well (200 μl/well of culture) in 96 well plates precoated with anti-CD3 antibody (BD Biocoat T-cell activation plates, Anti-human CD3 plate=Cat. No. 354725 from BD BioSciences). Anti-CD28 antibody (eBioScience, Cat# 16-0289-85; Functional Grade, co-stimulatory, clone 28.2) is added to the plate at the final concentration of 20 ng/well. Cells are incubated for 48 hrs after which a 50 μl aliquot of cell supernatant is removed for IL-2 titration. IL-2 titration was performed using the Endogen enzyme-linked immunosorbent assay (ELISA) Kit (Endogen of Pierce, Cat# EH2-IL2-5) as described by the manufacturer. In brief, this assay consists of using a 96 well microtiter plate that has been precoated with anti-IL-2 antibodies for the capture of human IL-2. In order to detect the captured IL-2, a biotinylated second anti-IL-2 antibody is added to all wells. This results in a sandwiching of any IL-2 (capture IL-2 Ab C IL-2 C detection IL-2-biotin Ab). Following the removal of unbound antibodies, by a series of washings, a horseradish peroxidase (HRP) conjugate with a high affinity for biotin is added. Unbound Strepavidin-HRP is removed and the bound enzyme labeled antibodies can then be measured via a chromogenic reaction with the addition of TMB substrate. The resulting chromogenic reaction is stopped using H₂SO₄ (Stop Solution) and the optical density of each well is then read at the appropriate wavelength. The level of substrate conversion is colormeterically determined by measuring the absorbance that is proportional to the amount of IL-2.

Representative compounds described herein demonstrated IL-2 inhibition.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

A number of embodiments of the invention have been described. Nevertheless, it will be understood that various modifications may be made without departing from the spirit and scope of the invention. Accordingly, other embodiments are within the scope of the following claims.

What is claimed is:

1. A compound of the formula (I) 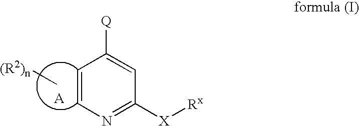
wherein;
A forms a benzene, pyridine, pyrimidine, thiophene, pyrrole, imidazole, pyrrazole, thiazole, or oxazole ring;
X is O, S, NR³, N(R³)N(R³), C(O), N(R⁵)C(O), C(O)NR⁵, or alkyl;
R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl wherein R^{X} is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when X—R^{X} is Me and Q is NHaryl substituted with heterocycyl; and wherein R² is not acetyl when X—R^{X} is arylalkenyl;
n is 0-3;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR⁵₂, NR⁵R⁹S-alkyl, alkyl, or heterocyclyl, wherein each alkyl or heterocyclyl is optionally substituted with 1-4 R¹⁰;
each R⁹ is independently aryl, heterocyclyl, heteroaryl, arylalkyl, or heteroarylalkyl, each of which is optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and
each R¹¹ is independently arylalkyl, heteroarylalkyl, cycloalkyl, or heterocyclyl.

2. The compound of claim 1, formula (I), wherein
A forms a benzene ring.

3. The compound of claim 1, formula (II) 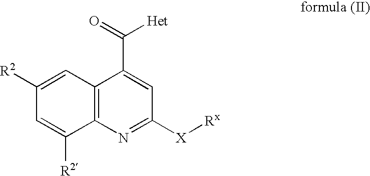
wherein;
X is O, S, NR³, N(R³)N(R³), C(O), N(R⁵)C(O), C(O)NR⁵, or alkyl;
R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷;
each R^{2′} is independently H, halo, NH₂, alkyl, OH, C(O)Me, aryl, heteroaryl;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Het is heterocyclyl optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and
each R¹¹ is independently arylalkyl, heteroarylalkyl, cycloalkyl, or heterocyclyl.

4. The compound of claim 3, formula (II), wherein
Het is attached through a ring-nitrogen atom.

5. The compound of claim 3, formula (II), wherein
R^{2′} is H.

6. The compound of claim 3, formula (II), wherein;
Het is attached through a ring-nitrogen atom;
X is NR³, or alkyl; and
R^{X} is cycloalkyl, aryl, heteroaryl, arylalkyl, heteroarylalkyl, or heterocyclyl wherein R^{X} is optionally substituted with 1-4 R⁴; and
R^{2′} is H.

7. The compound of claim 3, formula (II), wherein
Het is

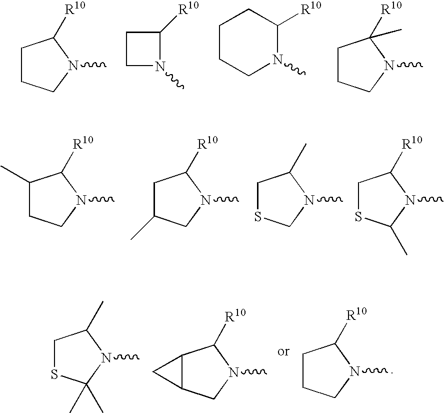

8. The compound of claim 3, formula (II), wherein
Het is

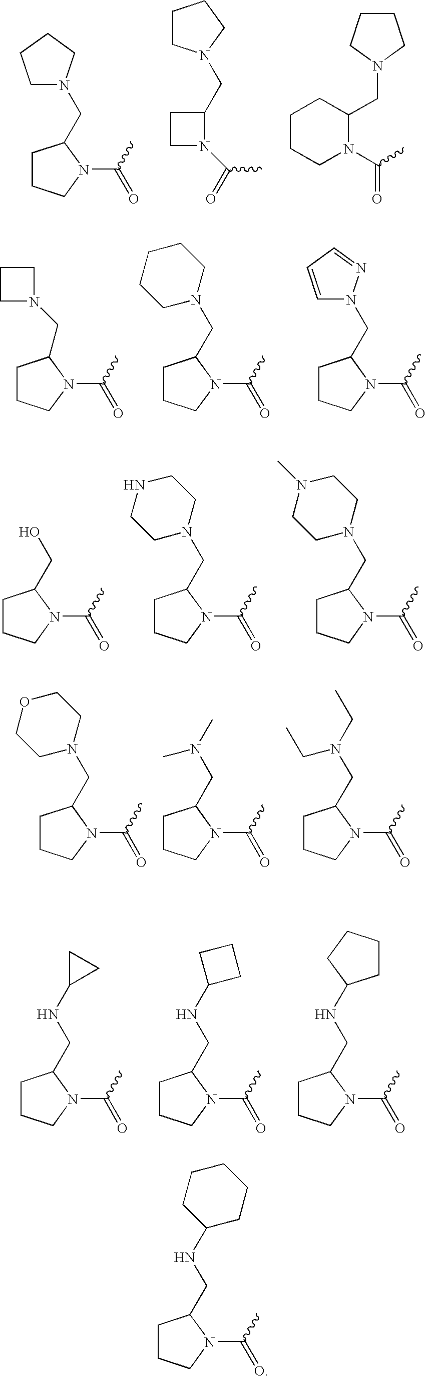

9. The compound of claim 1, formula (III), 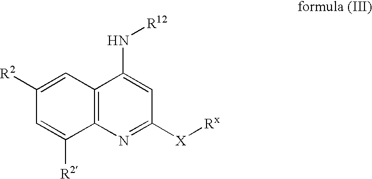
wherein,
X is O, S, NR³, N(R³)N(R³), C(O), N(R⁵)C(O), C(O)NR⁵, or alkyl;
R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when X—R^{X} is Me and R¹² is aryl substituted with heterocyclyl;
each R^{2′} is independently H, halo, NH₂, alkyl, OH, C(O)Me, aryl, heteroaryl;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
each R¹² is independently aryl or heteroaryl, optionally substituted with 1-4 R¹³; and
each R¹³ is independently heterocyclyl optionally substituted with alkyl or OR⁵.

10. The compound of claim 9 formula (III) wherein R^{2′} is H.

11. The compound of claim 9 formula (III), wherein
R¹² is

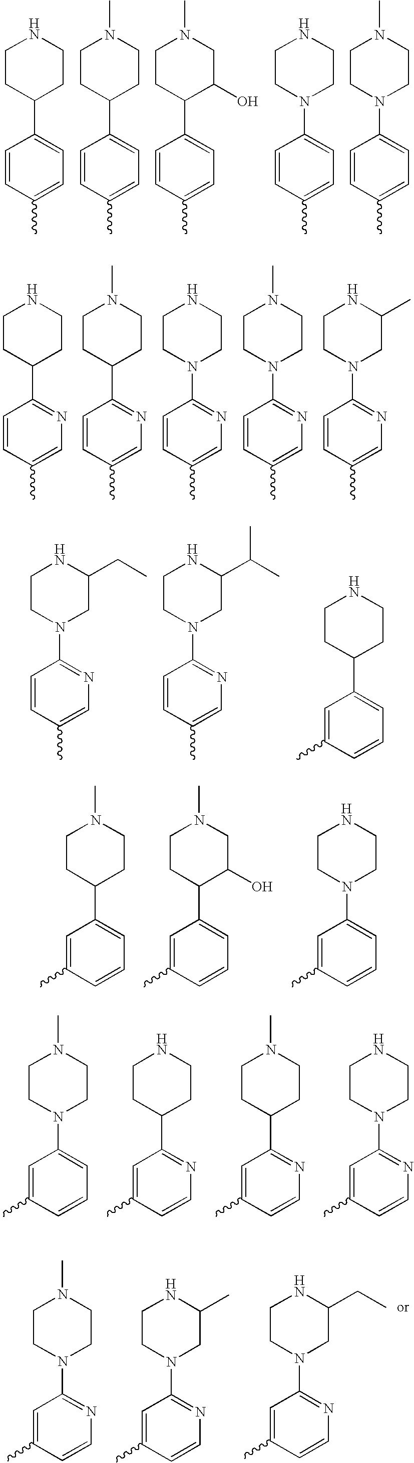

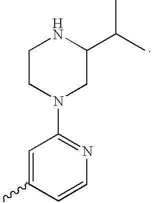

12. A compound of formula (IV) 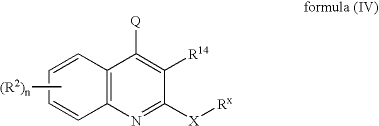
wherein,
X is O, S, NR³, N(R³)N(R³), C(O), N(R⁵)C(O), C(O)NR⁵, or alkyl;
R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, herein R^{X} is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when X—R^{X} is Me and Q is NHAryl substituted with heterocycyl; and wherein R² is not acetyl when X—R^{X} is arylalkenyl;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR¹², S(O)NR⁵R⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;
each R⁹ is independently aryl, heterocyclyl, heteroaryl, arylalkyl, or heteroarylalkyl, each of which is optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵;
each R¹¹ is independently arylalkyl, heteroarylalkyl, cycloalkyl, or heterocyclyl; and
R¹⁴ is halo, NH₂, alkyl, OH, C(O)Me, aryl, heteroaryl, or C(O)NHR⁵.

13. A compound of the formula (V) 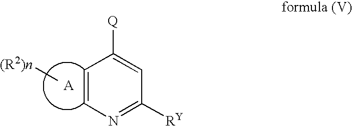
wherein;
A forms a benzene, pyridine, pyrimidine, thiophene, pyrrole, imidazole, pyrrazole, thiazole, or oxazole ring;
R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;
each R² is independently H, NR², alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when R^{Y} is halo, indole substituted with halo, phenyl, or phenyl substituted with halo; wherein R² is not isopropyl when R^{Y} is phenyl substituted with halo; wherein R² is not alkynyl substituted with heterocyclyl when R^{Y} is indole substituted with halo; wherein R² is not phenyl when R^{Y} is H, wherein R² is not halo when R^{Y} is indole substituted with halo or phenyl substituted with halo; and wherein R² is not acetyl when R^{Y} is phenyl or substituted phenyl;
n is 0-3;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or allyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;
each R⁹ is independently aryl heterocyclyl, heteroaryl, arylalkyl, or heteroarylalkyl, each of which is optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹, R⁵O-alkyl, R¹¹, heteroaryl, heterocyclyl, or heterocyclylalkyl, each of which is optionally substituted with alkyl or OR⁵; and
each R¹¹ is independently arylalkyl, heteroarylalkyl, cycloalkyl, or heterocyclyl.

14. The compound of claim 13, formula (V), wherein
A forms a benzene ring.

15. The compound of claim 13, formula (VI), wherein 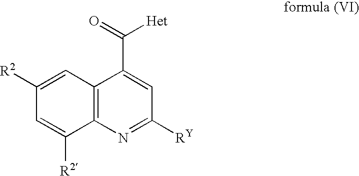
wherein,
R^{Y} is H, halo, aryl, heteroaryl, cycloalkyl, heterocyclyl, alkynyl, arylalkenyl, heteroarylalkenyl, arylalkynyl, or heteroarylalkynyl, wherein R¹ is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when R^{Y} is halo, indole substituted with halo, phenyl, or phenyl substituted with halo; wherein R² is not isopropyl when R^{Y} is phenyl substituted with halo;
each R^{2′} is independently H, halo, NH₂, alkyl, OH, C(O)Me, aryl, heteroaryl;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Het is heterocyclyl optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and
each R¹¹ is independently arylalkyl, heteroarylalkyl, cycloalkyl, or heterocyclyl.

16. The compound of claim 15, formula (VI), wherein
Het is attached through a ring-nitrogen atom;
R² is not H; and
R^{2′} is H.

17. The compound of claim 15, formula (VI), wherein
Het is

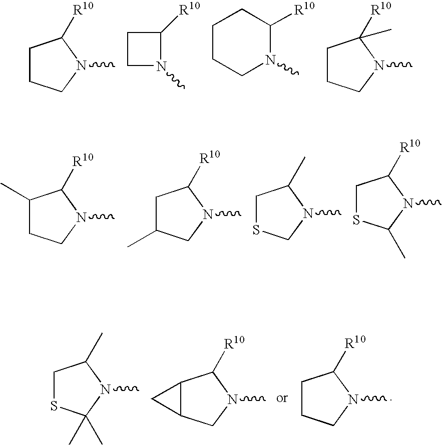

18. The compound of claim 1S, formula (VI), wherein
Het is

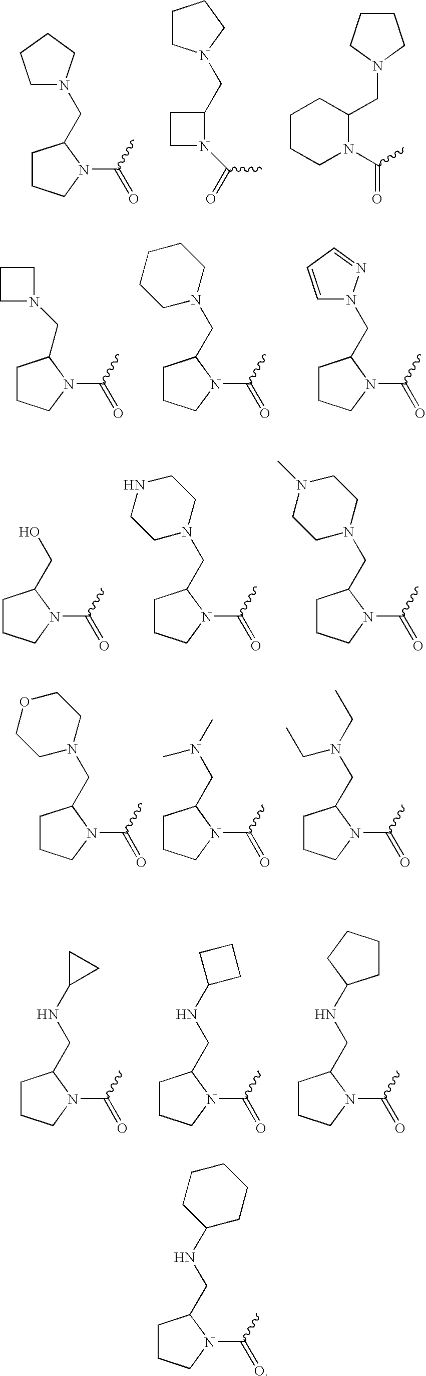

19. The compound of claim 13, formula (VII) 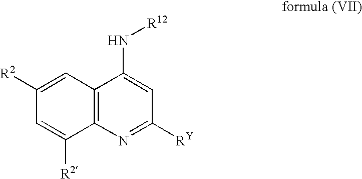
wherein;
R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not phenyl when R^{Y} is H;
each R^{2′} is independently H, halo, NH₂, alkyl, OH, C(O)Me, aryl, heteroaryl;
each R³ is independently H, alkyl, R⁵O-allyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
each R¹² is independently aryl or heteroaryl, optionally substituted with 1-4 R¹³; and
each R¹³ is independently heterocyclyl optionally substituted with alkyl or OR⁵.

20. The compound of claim 19 formula (VII), wherein
R² is not H; and
R^{2′} is H.

21. The compound of claim 19 formula (VII), wherein
R¹² is

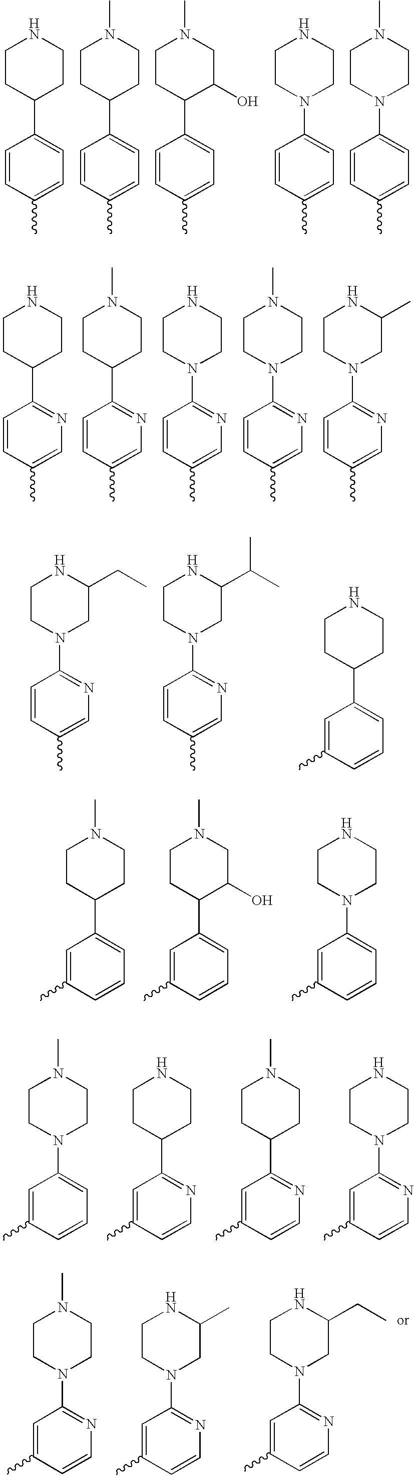

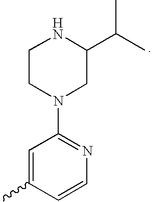

22. A compound of the formula (VIII), 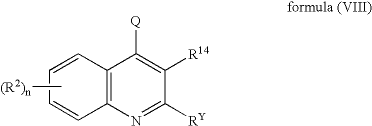
wherein,
R^{Y} is H, halo, aryl, heteroaryl, cycloalkyl, heterocyclyl, alkynyl, arylalkenyl, heteroarylalkenyl, arylalkynyl, or heteroarylalkynyl, wherein R^{Y} is optionally substituted with 1-4 R⁴;
each R² is independently H, NR⁵₂, alkyl, cycloalkyl, alkenyl, alkynyl, heteroaryl, heterocyclyl, aryl, halo, acetyl, arylalkyl, heteroarylalkyl, arylalkenyl, heteroarylalkenyl, arylalkynyl, heteroarylalkynyl, hydroxy, alkoxy, aryloxy, or heteroaryloxy, each of which is optionally substituted with 1-4 R⁷; wherein R² is not Me when R^{Y} is halo, indole substituted with halo, or phenyl optionally substituted with halo; wherein R² is not isopropyl when R^{Y} is phenyl substituted with halo; wherein R² is not alkynyl substituted with heterocyclyl when R^{Y} is indole substituted with halo; wherein R² is not phenyl when R^{Y} is H; and wherein R² is not acetyl when R^{Y} is phenyl or substituted phenyl;
each n is 0-3;
each R³ is independently H, alkyl, R⁵O-alkyl, or arylalkyl;
each R⁴ is independently halo, OH, CF₃, C(O)R⁵, NR⁵₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵R⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;
each R⁹ is independently aryl, heterocyclyl, heteroaryl, arylalkyl, or heteroarylalkyl, each of which is optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵;
each R¹¹ is independently arylalkyl heteroarylalkyl, cycloalkyl, or heterocyclyl; and
R¹⁴ is halo, NH₂, alkyl, OH, C(O)Me, aryl, heteroaryl, or C(O)NHR⁵.

23. The compound of claim 22, formula (VIE), wherein n is 1 and R² is not H.

24. The compound of formula (XIV), 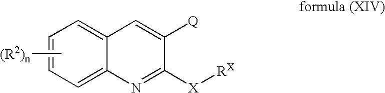
wherein,
X is O, S, NR³, N(R³)N(R³), C(O), N(R⁵)C(O), C(O)NR⁵, or alkyl;
R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;
each R² is independently H, NR⁵₂, alkyl, cycloalkyl, alkenyl, alkynyl, heteroaryl, heterocyclyl, aryl, halo, arylalkyl, heteroarylalkyl, arylalkenyl, heteroarylalkenyl, arylalkynyl, heteroarylalkynyl, aryloxy, or heteroaryloxy, each of which is optionally substituted with 1-4 R⁷;
n is 0-3;
each R³ is independently H, alkyl, R⁵O-alkyl, or arylalkyl;
each R⁴ is independently halo, OH, CF₃, C(O)R⁵, NR⁵₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Q is H, halo, C(O)R⁵, C(O)R⁹, C(S)R⁵, C(S)R⁹, C(O)NR⁵₂, C(O)NR⁵R⁹, S(O)R⁵, S(O)R⁹, S(O)NR⁵₂, S(O)NR⁵⁹, SO₂R⁵, SO₂R⁹, SO₂NR⁵₂, SO₂NR⁵R⁹, NR⁵₂, NR⁵R⁹, R⁹S-alkyl, alkyl, or heterocyclyl, wherein each alkyl or heterocyclyl is optionally substituted with 1-4 R¹⁰;
each R⁹ is independently aryl, heterocyclyl, heteroaryl, arylalkyl, or heteroarylalkyl, each of which is optionally substituted with 1-4 R¹⁰;
each R¹⁰ is independently alkyl, CF₃, C(NH)NR⁵R¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and
each R¹¹ is independently arylalkyl, heteroarylalkyl, cycloalkyl, or heterocyclyl.

25. A method of making a compound of formula (II) 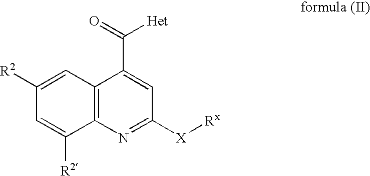
wherein X, R^{X}, R², R^{2′}, and Het are as defined below, the method comprising;
treating the compound of formula (IX) with malonic acid, to provide a ring expansion compound of formula (X); 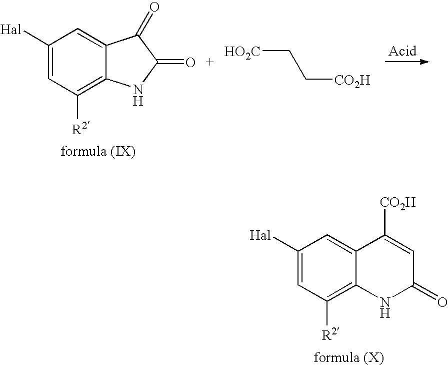
coupling the compound of formula (X) with a Pd catalyst and a compound of formula (XI) to provide a compound of formula (XII); 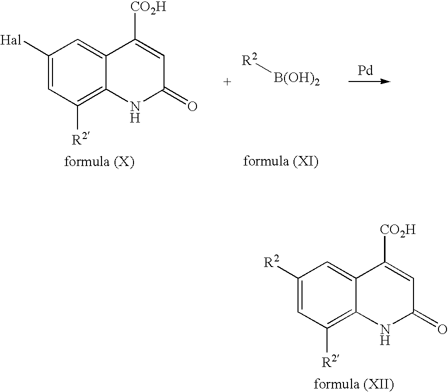
treating the compound of formula (XII) with POCl₃ to provide the chloride of formula (XIII), and coupling the carboxylic acid of formula (XII) with an amine of formula Het-H to provide the compound of formula (XIII); and 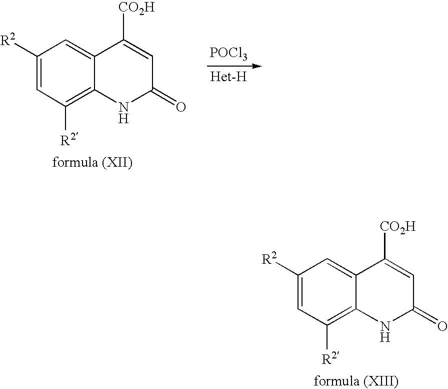
coupling the compound of formula (XIII) with one or more coupling agents 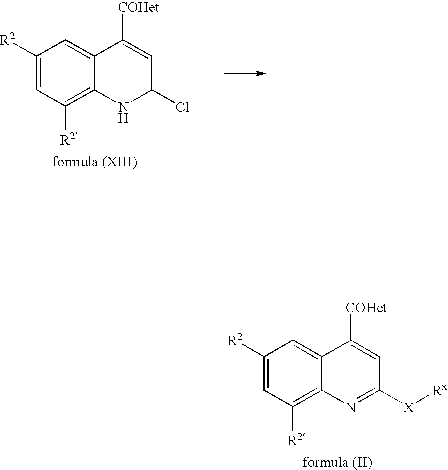
to provide a compound of formula (II), wherein for formulae II and IX to XIII,
X is O, S, NR³, N(R³)N(R³), C(O), N(R⁵)C(O)R⁵, C(O)NR⁵, or alkyl;
R^{X} is H, cycloalkyl, alkyl, heterocyclyl, aryl, heteroaryl, arylalkyl, heteroarylalkyl, alkynyl, arylalkenyl, heteroarylalkenyl, arylalkynyl, heteroarylalkynyl, R⁵O-alkyl, (R⁵)₃Si, acyl, wherein R^{X} is optionally substituted with 1-4 R⁴;
each R² is independently NR⁵₂, alkyl, cycloalkyl, alkenyl, alkynyl, heteroaryl, heterocyclyl, aryl, halo, arylalkyl, heteroarylalkyl, arylalkenyl, heteroarylalkenyl, arylalkynyl, heteroarylalkynyl, aryloxy, or heteroaryloxy, each of which is optionally substituted with 1-4 R⁷;
R^{2′} is H, halo, NH₂, alkyl, OH, C(O)Me, aryl, heteroaryl;
each R³ is independently H, alkyl, R⁵O-alkyl, or arylalkyl;
each R⁴ is independently halo, OH, CF₃, C(O)R⁵, NR³₂, N(R³)C(O)R⁵, CN, OCF₃, SO₂R⁵, or SiR⁵₃; or alkyloxy, aryloxy, alkyl, heterocyclyl, R⁵O-alkyl, cycloalkyl, aryl, alkylthio, haloalkyloxy, heteroaryl, or arylalkyl, each of which is optionally substituted with 1-4 R⁶;
each R⁵ is independently H, or alkyl;
each R⁶ is independently halo, OH, CF₃, alkyl, alkyloxy, N(R⁵)-alkyl, heteroaryl, heteroarylalkyl, or heterocyclyl;
each R⁷ is independently halo, CN, OR⁵, CF₃, N(R⁵)C(O)R⁵, C(O)R⁵, OCF₃, SCF₃, NR⁵₂, C(O)NR⁵₂, OH, R⁵O-alkyl, alkyl, alkylsulfonyl, heterocyclyl, or heteroaryl, each of which is optionally substituted with 1-4 R⁸;
each R⁸ is independently OR⁵ or alkyl;
Het is heterocyclyl optionally substituted with 1-4 R¹⁰;
each R¹⁰ is independently alkyl, CF₃, C(NH)NR⁵)NR¹¹, C(NH)R¹¹, CN, R⁵₂N-alkyl, NR⁵R¹¹-alkyl, R⁵O-alkyl, R¹¹, heteroaryl, heterocyclyl, or heterocyclylalkyl, each of which is optionally substituted with alkyl or OR⁵; and
each R¹¹ is independently arylalkyl, heteroarylalkyl, cycloalkyl, or heterocyclyl.

26. The method of claim 25, wherein the H of Het-H is attached to a nitrogen.

27. The method of claim 25, wherein the coupling agent is H₂NR^{X}.

28. The method of claim 25, wherein the coupling agents are MgCl—R^{X}, and Pd.

29. The method of claim 25, wherein the coupling agent is HSR^{X}.

30. A method of treating an autoimmune disorder in a subject comprising administering to the subject any of the compounds of claims 1-24.

31. The method of claim 30, further comprising administering an additional therapeutic agent.

32. The method of claim 30, wherein the autoimmune disorder is lupus.

33. A method of treating organ transplant rejection in a subject comprising administering to the subject any of the compounds of claims 1-24.

34. The method of claim 33, further comprising administering an additional therapeutic agent.

35. A method of treating an inflammatory disorder in a subject comprising administering to the subject any of the compounds of claims 1-24.

36. The method of claim 35, further comprising administering an additional therapeutic agent.

37. The method of claim 36, wherein the additional therapeutic agent is an analgesic, or a steroid.

38. The method of claim 35, wherein the inflammatory disorder is arthritis.

39. The method of claim 38, wherein the arthritis is rheumatoid arthritis, rheumatoid spondylitis, gouty arthritis, traumatic arthritis, rubella arthritis, psoriatic arthritis, or osteoarthritis.

40. The method of claim 35, wherein the inflammatory disorder is inflammatory bowel disease or Crohn's disease.

41. A composition comprising any of the compounds of claims 1-24.

42. The composition of claim 41, further comprising a pharmaceutically acceptable carrier.

43. The composition of claim 41, further comprising an additional therapeutic agent.

44. A library of the compounds of any of formulas (I)-(VIII).

45. A method of inhibiting IL-2 production in a subject comprising administering to the subject a compound of any of claims 1-24.

46. A method of modulating ZAP-70 in a subject comprising administering to the subject a compound of any of claims 1-24.
